SEQUENCE LISTING (91 sequences)

SEQ ID NO: 1 - protein, 469 aa, synthetic construct
MNPNQKIITIGSVSLTISTICFFMQIAILITTVTLHFKQYEFNSPPNNQVMLCEPTIIERNITEIVYLTNTTIEKEICPKPAEYRNWSKPQCDITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIEEGKVVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 2 - protein, 281 aa, Homo sapiens [sequence omitted]

SEQ ID NO: 3 - protein, 243 aa, Homo sapiens
TNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 4 - protein, 129 aa, Homo sapiens
SWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 5 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 6 - protein, 177 aa, Homo sapiens
GPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 7 - protein, 132 aa, Homo sapiens
LQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 8 - protein, 234 aa, Homo sapiens
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 9 - protein, 172 aa, Homo sapiens
QRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 10 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 11 - protein, 361 aa, Homo sapiens
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 12 - protein, 402 aa, Homo sapiens
DFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 13 - protein, 407 aa, Homo sapiens
KPLQSWGRGSAGGNAHSPLGVPGGGLPEHTFNLKMFLENVKVDFLRSLNLSGVPSQDKTRVEPPQYMIDLYNRYTSDKSTTPASNIVRSFSMEDAISITATEDFPFQKHILLFNISIPRHEQITRAELRLYVSCQNHVDPSHDLKGSVVIYDVLDGTDAWDSATETKTFLVSQDIQDEGWETLEVSSAVKRWVRSDSTKSKNKLEVTVESHRKGCDTLDISVPPGSRNLPFFVVFSNDHSSGTKETRLELREMISHEQESVLKKLSKDGSTEAGESSHEEDTDGHVAAGSTLARRKRSAGAGSHCQKTSLRVNFEDIGWDSWIIAPKEYEAYECKGGCFFPLADDVTPTKHAIVQTLVHLKFPTKVGKACCVPTKLSPISVLYKDDMGVPTLKYHYEGMSVAECGCR

SEQ ID NO: 14 - protein, 192 aa, Homo sapiens
FPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 15 - protein, 229 aa, Homo sapiens
APMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 16 - protein, 224 aa, Homo sapiens
QDEYMESPQTGGLPPDCSKCCHGDYSFRGYQGPPGPPGPPGIPGNHGNNGNNGATGHEGAKGEKGDKGDLGPRGERGQHGPKGEKGYPGIPPELQIAFMASLATHFSNQNSGIIFSSVETNIGNFFDVMTGRFGAPVSGVYFFTFSMMKHEDVEEVYVYLMHNGNTVFSMYSYEMKGKSDTSSNHAVLKLAKGDEVWLRMGNGALHGDHQRFSTFAGFLLFETK

SEQ ID NO: 17 - protein, 25 aa, synthetic construct
IFMYLLTVFLITQMIGSALFAVYLH

SEQ ID NO: 18 - protein, 52 aa, synthetic construct
MIETYNQTSPRSAATGLPISMKIFMYLLTVFLITQMIGSALFAVYLHRRLDK

SEQ ID NO: 19 - protein, 26 aa, synthetic construct
IYIWAPLAGICVALLLSLIITLICYH

SEQ ID NO: 20 - protein, 28 aa, synthetic construct
DIYIWAPLAGICVALLLSLIITLICYHR

SEQ ID NO: 21 - protein, 4 aa, synthetic construct
  REGION 1..4: note = Pilot peptide
YINL

SEQ ID NO: 22 - protein, 4 aa, synthetic construct
  REGION 1..4: note = Pilot peptide
YSHL

SEQ ID NO: 23 - protein, 4 aa, synthetic construct
  REGION 1..4: note = Pilot peptide
YINF

SEQ ID NO: 24 - protein, 4 aa, synthetic construct
  REGION 1..4: note = Pilot peptide
YSHF

SEQ ID NO: 25 - protein, 5 aa, synthetic construct
  REGION 1..5: note = Pilot peptide
DYINL

SEQ ID NO: 26 - protein, 5 aa, synthetic construct
  REGION 1..5: note = Pilot peptide
DYINF

SEQ ID NO: 27 - protein, 4 aa, synthetic construct
  REGION 1..4: note = Pilot peptide
PSAP

SEQ ID NO: 28 - protein, 4 aa, synthetic construct
  REGION 1..4: note = Pilot peptide
PTAP

SEQ ID NO: 29 - protein, 40 aa, synthetic construct
  REGION 1..40: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 10: note = X represents any amino acid residue
  VARIANT 11: note = X represents any amino acid residue
  VARIANT 13: note = X represents any amino acid residue
  VARIANT 15: note = X represents any amino acid residue
  VARIANT 17: note = X represents any amino acid residue
  VARIANT 18: note = X represents any amino acid residue
  VARIANT 20: note = X represents any amino acid residue
  VARIANT 22: note = X represents any amino acid residue
  VARIANT 23: note = X represents any amino acid residue
  VARIANT 26: note = X represents any amino acid residue
  VARIANT 28: note = X represents any amino acid residue
  VARIANT 29: note = X represents any amino acid residue
  VARIANT 31: note = X represents any amino acid residue
  VARIANT 33: note = X represents any amino acid residue
  VARIANT 34: note = X represents any amino acid residue
  VARIANT 38: note = X represents any amino acid residue
  VARIANT 39: note = X represents any amino acid residue
PXXPXXXXPXXPXSXYXXLXPXXPEXYXXLXPXXPDYXXL

SEQ ID NO: 30 - protein, 37 aa, synthetic construct
  REGION 1..37: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any amino acid residue
  VARIANT 8: note = X represents any amino acid residue
  VARIANT 10: note = X represents any one or several amino acid residue(s)
  VARIANT 12: note = X represents any amino acid residue
  VARIANT 14: note = X represents any amino acid residue
  VARIANT 15: note = X represents any amino acid residue
  VARIANT 17: note = X represents any one or several amino acid residue(s)
  VARIANT 19: note = X represents any amino acid residue
  VARIANT 20: note = X represents any amino acid residue
  VARIANT 23: note = X represents any one or several amino acid residue(s)
  VARIANT 25: note = X represents any amino acid residue
  VARIANT 26: note = X represents any amino acid residue
  VARIANT 28: note = X represents any one or several amino acid residue(s)
  VARIANT 30: note = X represents any amino acid residue
  VARIANT 31: note = X represents any amino acid residue
  VARIANT 35: note = X represents any amino acid residue
  VARIANT 36: note = X represents any amino acid residue
PXXPXPXXPXSXYXXLXPXXPEXYXXLXPXXPDYXXL

SEQ ID NO: 31 - protein, 44 aa, synthetic construct
  REGION 1..44: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 10: note = X represents any amino acid residue
  VARIANT 11: note = X represents any amino acid residue
  VARIANT 13: note = X represents any amino acid residue
  VARIANT 15: note = X represents any amino acid residue
  VARIANT 17: note = X represents any amino acid residue
  VARIANT 18: note = X represents any amino acid residue
  VARIANT 20: note = X represents any amino acid residue
  VARIANT 22: note = X represents any amino acid residue
  VARIANT 23: note = X represents any amino acid residue
  VARIANT 26: note = X represents any amino acid residue
  VARIANT 28: note = X represents any amino acid residue
  VARIANT 29: note = X represents any amino acid residue
  VARIANT 31: note = X represents any amino acid residue
  VARIANT 33: note = X represents any amino acid residue
  VARIANT 34: note = X represents any amino acid residue
  VARIANT 38: note = X represents any amino acid residue
  VARIANT 39: note = X represents any amino acid residue
  VARIANT 41: note = X represents any amino acid residue
  VARIANT 42: note = X represents any amino acid residue
  VARIANT 43: note = X represents any amino acid residue
  VARIANT 44: note = X represents any amino acid residue
PXXPXXXXPXXPXSXYXXLXPXXPEXYXXLXPXXPDYXXLXXXX

SEQ ID NO: 32 - protein, 41 aa, synthetic construct
  REGION 1..41: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any amino acid residue
  VARIANT 8: note = X represents any amino acid residue
  VARIANT 10: note = X represents any one or several amino acid residue(s)
  VARIANT 12: note = X represents any amino acid residue
  VARIANT 15: note = X represents any amino acid residue
  VARIANT 17: note = X represents any one or several amino acid residue(s)
  VARIANT 19: note = X represents any amino acid residue
  VARIANT 20: note = X represents any amino acid residue
  VARIANT 23: note = X represents any one or several amino acid residue(s)
  VARIANT 25: note = X represents any amino acid residue
  VARIANT 26: note = X represents any amino acid residue
  VARIANT 28: note = X represents any one or several amino acid residue(s)
  VARIANT 30: note = X represents any amino acid residue
  VARIANT 31: note = X represents any amino acid residue
  VARIANT 35: note = X represents any amino acid residue
  VARIANT 36: note = X represents any amino acid residue
  VARIANT 38: note = X represents any amino acid residue
  VARIANT 39: note = X represents any amino acid residue
  VARIANT 40: note = X represents any amino acid residue
  VARIANT 41: note = X represents any amino acid residue
  VARIANT 14: note = X represents any amino acid residue
PXXPXPXXPXSXYXXLXPXXPEXYXXLXPXXPDYXXLXXXX

SEQ ID NO: 33 - protein, 20 aa, synthetic construct
  REGION 1..20: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 9: note = X represents any amino acid residue
  VARIANT 10: note = X represents any amino acid residue
  VARIANT 11: note = X represents any amino acid residue
  VARIANT 12: note = X represents any amino acid residue
  VARIANT 13: note = X represents any amino acid residue
  VARIANT 14: note = X represents any amino acid residue
  VARIANT 15: note = X represents any amino acid residue
  VARIANT 16: note = X represents any amino acid residue
  VARIANT 18: note = X represents any amino acid residue
  VARIANT 19: note = X represents any amino acid residue
PXXPXXXXXXXXXXXXYXXL

SEQ ID NO: 34 - protein, 20 aa, synthetic construct
  REGION 1..20: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 9: note = X represents any amino acid residue
  VARIANT 10: note = X represents any amino acid residue
  VARIANT 11: note = X represents any amino acid residue
  VARIANT 12: note = X represents any amino acid residue
  VARIANT 13: note = X represents any amino acid residue
  VARIANT 14: note = X represents any amino acid residue
  VARIANT 15: note = X represents any amino acid residue
  VARIANT 18: note = X represents any amino acid residue
  VARIANT 19: note = X represents any amino acid residue
PXXPXXXXXXXXXXXDYXXL

SEQ ID NO: 35 - protein, 20 aa, synthetic construct
  REGION 1..20: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 8: note = X represents any amino acid residue
  VARIANT 9: note = X represents any amino acid residue
  VARIANT 10: note = X represents any amino acid residue
  VARIANT 11: note = X represents any amino acid residue
  VARIANT 12: note = X represents any amino acid residue
  VARIANT 13: note = X represents any amino acid residue
  VARIANT 14: note = X represents any amino acid residue
  VARIANT 15: note = X represents any amino acid residue
  VARIANT 16: note = X represents any amino acid residue
  VARIANT 18: note = X represents any amino acid residue
  VARIANT 19: note = X represents any amino acid residue
PXXPXXYXXXXXXXXXYXXL

SEQ ID NO: 36 - protein, 20 aa, synthetic construct
  REGION 1..20: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 8: note = X represents any amino acid residue
  VARIANT 9: note = X represents any amino acid residue
  VARIANT 10: note = X represents any amino acid residue
  VARIANT 11: note = X represents any amino acid residue
  VARIANT 12: note = X represents any amino acid residue
  VARIANT 13: note = X represents any amino acid residue
  VARIANT 14: note = X represents any amino acid residue
  VARIANT 15: note = X represents any amino acid residue
  VARIANT 18: note = X represents any amino acid residue
  VARIANT 19: note = X represents any amino acid residue
PXXPXXYXXXXXXXXDYXXL

SEQ ID NO: 37 - protein, 20 aa, synthetic construct
  REGION 1..20: note = Pilot peptide
  VARIANT 2: note = X represents any amino acid residue
  VARIANT 3: note = X represents any amino acid residue
  VARIANT 6: note = X represents any amino acid residue
  VARIANT 8: note = X represents any amino acid residue
  VARIANT 9: note = X represents any amino acid residue
  VARIANT 11: note = X represents any amino acid residue
  VARIANT 13: note = X represents any amino acid residue
  VARIANT 14: note = X represents any amino acid residue
  VARIANT 18: note = X represents any amino acid residue
  VARIANT 19: note = X represents any amino acid residue
PXXPEXYXXLXPXXPDYXXL

SEQ ID NO: 38 - protein, 9 aa, synthetic construct
  REGION 1..9: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any amino acid residue
  VARIANT 8: note = X represents any amino acid residue
PXXPXYXXL

SEQ ID NO: 39 - protein, 10 aa, synthetic construct
  REGION 1..10: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 8: note = X represents any amino acid residue
  VARIANT 9: note = X represents any amino acid residue
PXXPXDYXXL

SEQ ID NO: 40 - protein, 11 aa, synthetic construct
  REGION 1..11: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any one or several amino acid residue(s)
  VARIANT 9: note = X represents any amino acid residue
  VARIANT 10: note = X represents any amino acid residue
PXXPXYXYXXL

SEQ ID NO: 41 - protein, 12 aa, synthetic construct
  REGION 1..12: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any one or several amino acid residue(s)
  VARIANT 10: note = X represents any amino acid residue
  VARIANT 11: note = X represents any amino acid residue
PXXPXYXDYXXL

SEQ ID NO: 42 - protein, 20 aa, synthetic construct
  REGION 1..20: note = Pilot peptide
  VARIANT 2: note = X represents any amino acid residue
  VARIANT 3: note = X represents any amino acid residue
  VARIANT 6: note = X represents any one or several amino acid residue(s)
  VARIANT 8: note = X represents any amino acid residue
  VARIANT 9: note = X represents any amino acid residue
  VARIANT 11: note = X represents any one or several amino acid residue(s)
  VARIANT 13: note = X represents any amino acid residue
  VARIANT 14: note = X represents any amino acid residue
  VARIANT 18: note = X represents any amino acid residue
  VARIANT 19: note = X represents any amino acid residue
PXXPEXYXXLXPXXPDYXXL

SEQ ID NO: 43 - protein, 40 aa, synthetic construct
  REGION 1..40: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 10: note = X represents any amino acid residue
  VARIANT 11: note = X represents any amino acid residue
  VARIANT 13: note = X represents any amino acid residue
  VARIANT 14: note = X represents any amino acid residue
  VARIANT 15: note = X represents any amino acid residue
  VARIANT 17: note = X represents any amino acid residue
  VARIANT 18: note = X represents any amino acid residue
  VARIANT 20: note = X represents any amino acid residue
  VARIANT 22: note = X represents any amino acid residue
  VARIANT 23: note = X represents any amino acid residue
  VARIANT 26: note = X represents any amino acid residue
  VARIANT 28: note = X represents any amino acid residue
  VARIANT 29: note = X represents any amino acid residue
  VARIANT 31: note = X represents any amino acid residue
  VARIANT 33: note = X represents any amino acid residue
  VARIANT 34: note = X represents any amino acid residue
  VARIANT 38: note = X represents any amino acid residue
  VARIANT 39: note = X represents any amino acid residue
PXXPXXXXPXXPXXXYXXLXPXXPEXYXXLXPXXPDYXXL

SEQ ID NO: 44 - protein, 35 aa, synthetic construct
  REGION 1..35: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any amino acid residue
  VARIANT 8: note = X represents any amino acid residue
  VARIANT 10: note = X represents any one or several amino acid residue(s)
  VARIANT 12: note = X represents any amino acid residue
  VARIANT 13: note = X represents any amino acid residue
  VARIANT 15: note = X represents any one or several amino acid residue(s)
  VARIANT 17: note = X represents any amino acid residue
  VARIANT 18: note = X represents any amino acid residue
  VARIANT 21: note = X represents any one or several amino acid residue(s)
  VARIANT 23: note = X represents any amino acid residue
  VARIANT 24: note = X represents any amino acid residue
  VARIANT 26: note = X represents any one or several amino acid residue(s)
  VARIANT 28: note = X represents any amino acid residue
  VARIANT 29: note = X represents any amino acid residue
  VARIANT 33: note = X represents any amino acid residue
  VARIANT 34: note = X represents any amino acid residue
PXXPXPXXPXYXXLXPXXPEXYXXLXPXXPDYXXL

SEQ ID NO: 45 - protein, 44 aa, synthetic construct
  REGION 1..44: note = Pilot peptide
  VARIANT 2: note = X represents any amino acid residue
  VARIANT 3: note = X represents any amino acid residue
  VARIANT 5: note = X represents any amino acid residue
  VARIANT 6: note = X represents any amino acid residue
  VARIANT 7: note = X represents any amino acid residue
  VARIANT 8: note = X represents any amino acid residue
  VARIANT 10: note = X represents any amino acid residue
  VARIANT 11: note = X represents any amino acid residue
  VARIANT 13: note = X represents any amino acid residue
  VARIANT 14: note = X represents any amino acid residue
  VARIANT 15: note = X represents any amino acid residue
  VARIANT 17: note = X represents any amino acid residue
  VARIANT 18: note = X represents any amino acid residue
  VARIANT 20: note = X represents any amino acid residue
  VARIANT 22: note = X represents any amino acid residue
  VARIANT 23: note = X represents any amino acid residue
  VARIANT 26: note = X represents any amino acid residue
  VARIANT 28: note = X represents any amino acid residue
  VARIANT 29: note = X represents any amino acid residue
  VARIANT 31: note = X represents any amino acid residue
  VARIANT 33: note = X represents any amino acid residue
  VARIANT 34: note = X represents any amino acid residue
  VARIANT 38: note = X represents any amino acid residue
  VARIANT 39: note = X represents any amino acid residue
  VARIANT 41: note = X represents any amino acid residue
  VARIANT 42: note = X represents any amino acid residue
  VARIANT 43: note = X represents any amino acid residue
  VARIANT 44: note = X represents any amino acid residue
PXXPXXXXPXXPXXXYXXLXPXXPEXYXXLXPXXPDYXXLXXXX

SEQ ID NO: 46 - protein, 39 aa, synthetic construct
  REGION 1..39: note = Pilot peptide
  VARIANT 2: note = X represents any amino acid residue
  VARIANT 3: note = X represents any amino acid residue
  VARIANT 5: note = X represents any one or several amino acid residue(s)
  VARIANT 7: note = X represents any amino acid residue
  VARIANT 8: note = X represents any amino acid residue
  VARIANT 10: note = X represents any one or several mino acid residue(s)
  VARIANT 12: note = X represents any amino acid residue
  VARIANT 13: note = X represents any amino acid residue
  VARIANT 15: note = X represents any one or several amino acid residue(s)
  VARIANT 17: note = X represents any amino acid residue
  VARIANT 18: note = X represents any amino acid residue
  VARIANT 21: note = X represents any one or several amino acid residue(s)
  VARIANT 23: note = X represents any amino acid residue
  VARIANT 24: note = X represents any amino acid residue
  VARIANT 26: note = X represents any one or several amino acid residue(s)
  VARIANT 28: note = X represents any amino acid residue
  VARIANT 29: note = X represents any amino acid residue
  VARIANT 33: note = X represents any amino acid residue
  VARIANT 34: note = X represents any amino acid residue
  VARIANT 36: note = X represents any amino acid residue
  VARIANT 37: note = X represents any amino acid residue
  VARIANT 38: note = X represents any amino acid residue
  VARIANT 39: note = X represents any amino acid residue
PXXPXPXXPXYXXLXPXXPEXYXXLXPXXPDYXXLXXXX

SEQ ID NO: 47 - protein, 45 aa, synthetic construct
  REGION 1..45: note = Pilot peptide
APHFPEISFPPKPDSDYQALLPSAPEIYSHLSPTKPDYINLRPAP

SEQ ID NO: 48 - protein, 4 aa, synthetic construct
  REPEAT 1..4: note = Repeat from 1 to 8 times
  REGION 1..4: note = linker
GGGS

SEQ ID NO: 49 - protein, 5 aa, synthetic construct
  REPEAT 1..5: note = Repeat from 1 to 8 times
  REGION 1..5: note = linker
GGGGS

SEQ ID NO: 50 - protein, 6 aa, synthetic construct
  REPEAT 1..6: note = Repeat from 1 to 8 times
  REGION 1..6: note = linker
GGGGGS

SEQ ID NO: 51 - protein, 9 aa, synthetic construct
GGGSGGGGS

SEQ ID NO: 52 - protein, 33 aa, synthetic construct
SGGGSGGGGSGGGSGGGGSGGGSGGGGSGGGGS

SEQ ID NO: 53 - protein, 32 aa, synthetic construct
GGGSGGGGSGGGSGGGGSGGGSGGGGSGGGSG

SEQ ID NO: 54 - skipped

SEQ ID NO: 55 - protein, 6 aa, synthetic construct
  VARIANT 1: note = M is either present or, when located at the N terminal extremity, can be removed in vivo by post-translation processing
  VARIANT 3..4: note = X represents any amino acid residue
MGXXKS

SEQ ID NO: 56 - protein, 11 aa, synthetic construct
  VARIANT 3: note = X represents any amino acid residue
  VARIANT 5..8: note = X represents any amino acid residue
CKXKXXXXRRR

SEQ ID NO: 57 - protein, 16 aa, synthetic construct
  VARIANT 1: note = M is either present or, when located at the N terminal extremity, can be removed in vivo by post-translation processing
  REGION 1..16: note = sub-membrane targeting domain
MGSSKSKPKDPSQRRR

SEQ ID NO: 58 - protein, 19 aa, synthetic construct
  VARIANT 1: note = M is either present or, when located at the N terminal extremity, can be removed in vivo by post-translation processing
  REGION 1..19: note = sub-membrane targeting domain
MGSSKSKPKDPSQRRRKSR

SEQ ID NO: 59 - protein, 23 aa, synthetic construct
  VARIANT 1: note = M is either present or, when located at the N terminal extremity, can be removed in vivo by post-translation processing
  REGION 1..23: note = sub-membrane targeting domain
MGSSKSKPKDPSQRRRKSRGPGG

SEQ ID NO: 60 - protein, 21 aa, synthetic construct
MGSSKSKPKDPSQRRRKSRGP

SEQ ID NO: 61 - protein, 57 aa, synthetic construct
MSRGGGAPHFPEISFPPKPDSDYQALLPSAPEIYSHLSPTKPDYINLRPAPGGSRAT

SEQ ID NO: 62 - protein, 41 aa, synthetic construct
MGSSKSKPKDPSQRRRKSRGPGGGSGGGSGGGSGGGSSRAT

SEQ ID NO: 63 - protein, 93 aa, synthetic construct
MGSSKSKPKDPSQRRRKSRGPGGGSGGGSGGGSGGGSSRGGGAPHFPEISFPPKPDSDYQALLPSAPEIYSHLSPTKPDYINLRPAPGGSRAT

SEQ ID NO: 64 - protein, 109 aa, synthetic construct
SGGGSGGGGSGGGSGGGGSGGGSGGGGSGGGGSDIYIWAPLAGICVALLLSLIITLICYHRSRGAPHFPEISFPPKPDSDYQALLPSAPEIYSHLSPTKPDYINLRPAP

SEQ ID NO: 65 - protein, 141 aa, synthetic construct
MSRGGGAPHFPEISFPPKPDSDYQALLPSAPEIYSHLSPTKPDYINLRPAPGGSRGSMIETYNQTSPRSAATGLPISMKIFMYLLTVFLITQMIGSALFAVYLHRRLDKGGGSGGGGSGGGSGGGGSGGGSGGGGSGGGSG

SEQ ID NO: 66 - protein, 125 aa, synthetic construct
MGSSKSKPKDPSQRRRKSRGPGGGSGGGSGGGSGGGSSRGSMIETYNQTSPRSAATGLPISMKIFMYLLTVFLITQMIGSALFAVYLHRRLDKGGGSGGGGSGGGSGGGGSGGGSGGGGSGGGSG

SEQ ID NO: 67 - protein, 177 aa, synthetic construct
MGSSKSKPKDPSQRRRKSRGPGGGSGGGSGGGSGGGSSRGGGAPHFPEISFPPKPDSDYQALLPSAPEIYSHLSPTKPDYINLRPAPGGSRGSMIETYNQTSPRSAATGLPISMKIFMYLLTVFLITQMIGSALFAVYLHRRLDKGGGSGGGGSGGGSGGGGSGGGSGGGGSGGGSG

SEQ ID NO: 68 - protein, 526 aa, synthetic construct
MSRGGGAPHFPEISFPPKPDSDYQALLPSAPEIYSHLSPTKPDYINLRPAPGGSRATMNPNQKIITIGSVSLTISTICFFMQIAILITTVTLHFKQYEFNSPPNNQVMLCEPTIIERNITEIVYLTNTTIEKEICPKPAEYRNWSKPQCDITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIEEGKVVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 69 - protein, 338 aa, synthetic construct
MSRGGGAPHFPEISFPPKPDSDYQALLPSAPEIYSHLSPTKPDYINLRPAPGGSRATMAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 70 - protein, 290 aa, synthetic construct
MSRGGGAPHFPEISFPPKPDSDYQALLPSAPEIYSHLSPTKPDYINLRPAPGGSRATMSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 71 - protein, 291 aa, synthetic construct
MSRGGGAPHFPEISFPPKPDSDYQALLPSAPEIYSHLSPTKPDYINLRPAPGGSRATMDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 72 - protein, 311 aa, synthetic construct
MSRGGGAPHFPEISFPPKPDSDYQALLPSAPEIYSHLSPTKPDYINLRPAPGGSRATMEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 73 - protein, 295 aa, synthetic construct
MGSSKSKPKDPSQRRRKSRGPGGGSGGGSGGGSGGGSSRATMEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 74 - protein, 347 aa, synthetic construct
MGSSKSKPKDPSQRRRKSRGPGGGSGGGSGGGSGGGSSRGGGAPHFPEISFPPKPDSDYQALLPSAPEIYSHLSPTKPDYINLRPAPGGSRATMEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 75 - protein, 333 aa, synthetic construct
QDEYMESPQTGGLPPDCSKCCHGDYSFRGYQGPPGPPGPPGIPGNHGNNGNNGATGHEGAKGEKGDKGDLGPRGERGQHGPKGEKGYPGIPPELQIAFMASLATHFSNQNSGIIFSSVETNIGNFFDVMTGRFGAPVSGVYFFTFSMMKHEDVEEVYVYLMHNGNTVFSMYSYEMKGKSDTSSNHAVLKLAKGDEVWLRMGNGALHGDHQRFSTFAGFLLFETKSGGGSGGGGSGGGSGGGGSGGGSGGGGSGGGGSDIYIWAPLAGICVALLLSLIITLICYHRSRGAPHFPEISFPPKPDSDYQALLPSAPEIYSHLSPTKPDYINLRPAP

SEQ ID NO: 76 - protein, 384 aa, synthetic construct
MSRGGGAPHFPEISFPPKPDSDYQALLPSAPEIYSHLSPTKPDYINLRPAPGGSRGSMIETYNQTSPRSAATGLPISMKIFMYLLTVFLITQMIGSALFAVYLHRRLDKGGGSGGGGSGGGSGGGGSGGGSGGGGSGGGSGTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 77 - protein, 270 aa, synthetic construct
MSRGGGAPHFPEISFPPKPDSDYQALLPSAPEIYSHLSPTKPDYINLRPAPGGSRGSMIETYNQTSPRSAATGLPISMKIFMYLLTVFLITQMIGSALFAVYLHRRLDKGGGSGGGGSGGGSGGGGSGGGSGGGGSGGGSGSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 78 - protein, 318 aa, synthetic construct
MSRGGGAPHFPEISFPPKPDSDYQALLPSAPEIYSHLSPTKPDYINLRPAPGGSRGSMIETYNQTSPRSAATGLPISMKIFMYLLTVFLITQMIGSALFAVYLHRRLDKGGGSGGGGSGGGSGGGGSGGGSGGGGSGGGSGGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 79 - protein, 273 aa, synthetic construct
MSRGGGAPHFPEISFPPKPDSDYQALLPSAPEIYSHLSPTKPDYINLRPAPGGSRGSMIETYNQTSPRSAATGLPISMKIFMYLLTVFLITQMIGSALFAVYLHRRLDKGGGSGGGGSGGGSGGGGSGGGSGGGGSGGGSGLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 80 - protein, 313 aa, synthetic construct
MSRGGGAPHFPEISFPPKPDSDYQALLPSAPEIYSHLSPTKPDYINLRPAPGGSRGSMIETYNQTSPRSAATGLPISMKIFMYLLTVFLITQMIGSALFAVYLHRRLDKGGGSGGGGSGGGSGGGGSGGGSGGGGSGGGSGQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 81 - protein, 502 aa, synthetic construct
MSRGGGAPHFPEISFPPKPDSDYQALLPSAPEIYSHLSPTKPDYINLRPAPGGSRGSMIETYNQTSPRSAATGLPISMKIFMYLLTVFLITQMIGSALFAVYLHRRLDKGGGSGGGGSGGGSGGGGSGGGSGGGGSGGGSGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 82 - protein, 543 aa, synthetic construct
MSRGGGAPHFPEISFPPKPDSDYQALLPSAPEIYSHLSPTKPDYINLRPAPGGSRGSMIETYNQTSPRSAATGLPISMKIFMYLLTVFLITQMIGSALFAVYLHRRLDKGGGSGGGGSGGGSGGGGSGGGSGGGGSGGGSGDFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 83 - protein, 548 aa, synthetic construct
MSRGGGAPHFPEISFPPKPDSDYQALLPSAPEIYSHLSPTKPDYINLRPAPGGSRGSMIETYNQTSPRSAATGLPISMKIFMYLLTVFLITQMIGSALFAVYLHRRLDKGGGSGGGGSGGGSGGGGSGGGSGGGGSGGGSGKPLQSWGRGSAGGNAHSPLGVPGGGLPEHTFNLKMFLENVKVDFLRSLNLSGVPSQDKTRVEPPQYMIDLYNRYTSDKSTTPASNIVRSFSMEDAISITATEDFPFQKHILLFNISIPRHEQITRAELRLYVSCQNHVDPSHDLKGSVVIYDVLDGTDAWDSATETKTFLVSQDIQDEGWETLEVSSAVKRWVRSDSTKSKNKLEVTVESHRKGCDTLDISVPPGSRNLPFFVVFSNDHSSGTKETRLELREMISHEQESVLKKLSKDGSTEAGESSHEEDTDGHVAAGSTLARRKRSAGAGSHCQKTSLRVNFEDIGWDSWIIAPKEYEAYECKGGCFFPLADDVTPTKHAIVQTLVHLKFPTKVGKACCVPTKLSPISVLYKDDMGVPTLKYHYEGMSVAECGCR

SEQ ID NO: 84 - protein, 333 aa, synthetic construct
MSRGGGAPHFPEISFPPKPDSDYQALLPSAPEIYSHLSPTKPDYINLRPAPGGSRGSMIETYNQTSPRSAATGLPISMKIFMYLLTVFLITQMIGSALFAVYLHRRLDKGGGSGGGGSGGGSGGGGSGGGSGGGGSGGGSGFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 85 - protein, 370 aa, synthetic construct
MSRGGGAPHFPEISFPPKPDSDYQALLPSAPEIYSHLSPTKPDYINLRPAPGGSRGSMIETYNQTSPRSAATGLPISMKIFMYLLTVFLITQMIGSALFAVYLHRRLDKGGGSGGGGSGGGSGGGGSGGGSGGGGSGGGSGAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 86 - protein, 365 aa, synthetic construct
MSRGGGAPHFPEISFPPKPDSDYQALLPSAPEIYSHLSPTKPDYINLRPAPGGSRGSMIETYNQTSPRSAATGLPISMKIFMYLLTVFLITQMIGSALFAVYLHRRLDKGGGSGGGGSGGGSGGGGSGGGSGGGGSGGGSGQDEYMESPQTGGLPPDCSKCCHGDYSFRGYQGPPGPPGPPGIPGNHGNNGNNGATGHEGAKGEKGDKGDLGPRGERGQHGPKGEKGYPGIPPELQIAFMASLATHFSNQNSGIIFSSVETNIGNFFDVMTGRFGAPVSGVYFFTFSMMKHEDVEEVYVYLMHNGNTVFSMYSYEMKGKSDTSSNHAVLKLAKGDEVWLRMGNGALHGDHQRFSTFAGFLLFETK

SEQ ID NO: 87 - protein, 349 aa, synthetic construct
MGSSKSKPKDPSQRRRKSRGPGGGSGGGSGGGSGGGSSRGSMIETYNQTSPRSAATGLPISMKIFMYLLTVFLITQMIGSALFAVYLHRRLDKGGGSGGGGSGGGSGGGGSGGGSGGGGSGGGSGQDEYMESPQTGGLPPDCSKCCHGDYSFRGYQGPPGPPGPPGIPGNHGNNGNNGATGHEGAKGEKGDKGDLGPRGERGQHGPKGEKGYPGIPPELQIAFMASLATHFSNQNSGIIFSSVETNIGNFFDVMTGRFGAPVSGVYFFTFSMMKHEDVEEVYVYLMHNGNTVFSMYSYEMKGKSDTSSNHAVLKLAKGDEVWLRMGNGALHGDHQRFSTFAGFLLFETK

SEQ ID NO: 88 - protein, 401 aa, synthetic construct
MGSSKSKPKDPSQRRRKSRGPGGGSGGGSGGGSGGGSSRGGGAPHFPEISFPPKPDSDYQALLPSAPEIYSHLSPTKPDYINLRPAPGGSRGSMIETYNQTSPRSAATGLPISMKIFMYLLTVFLITQMIGSALFAVYLHRRLDKGGGSGGGGSGGGSGGGGSGGGSGGGGSGGGSGQDEYMESPQTGGLPPDCSKCCHGDYSFRGYQGPPGPPGPPGIPGNHGNNGNNGATGHEGAKGEKGDKGDLGPRGERGQHGPKGEKGYPGIPPELQIAFMASLATHFSNQNSGIIFSSVETNIGNFFDVMTGRFGAPVSGVYFFTFSMMKHEDVEEVYVYLMHNGNTVFSMYSYEMKGKSDTSSNHAVLKLAKGDEVWLRMGNGALHGDHQRFSTFAGFLLFETK

SEQ ID NO: 89 - protein, 4 aa, synthetic construct
PPPP

SEQ ID NO: 90 - protein, 183 aa, synthetic construct
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 91 - protein, 240 aa, synthetic construct
MSRGGGAPHFPEISFPPKPDSDYQALLPSAPEIYSHLSPTKPDYINLRPAPGGSRATMERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

(19) United States
(12) Patent Application Publication
(10) Pub. No.: US 2026/0295068 A1
(43) Pub. Date: Oct. 1, 2026

(54) CHIMERIC POLYPEPTIDES, EXTRACELLULAR VESICLES COMPRISING THE SAME, AND USES THEREOF

(71) Applicants: CILOA, Montpellier (FR); JERID, Saint-André-de Sangonis (FR)

(72) Inventors: Zaïne El Abiddine Robert Mamoun, Saint-Andre-de Sangonis (FR); Bernadetie Nadine Trentin, Montpellier (FR)

(21) Appl. No.: 19/489,845

(22) PCT Filed: Jun. 5, 2024
(86) PCT No.: PCT/EP2024/065475

§ 371 Date: Dec. 3, 2025

Related U.S. Application Data
(60) Provisional application No. 63/506,213, filed on Jun. 5, 2023.

(51) Int. Cl. A61K 47/69; A61K 38/00; C07K 14/705; C12N 9/24

(52) U.S. Cl. CPC A61K 47/6901; C07K 14/70517; C07K 14/70575; C12N 9/2402; A61K 38/00; C07K 2319/03; C07K 2319/70; C12Y 302/01018

(57) ABSTRACT

The present invention relates to chimeric polypeptides, nucleic acids encoding said chimeric polypeptides, and extracellular vesicles comprising said chimeric polypeptides. It further relates to the use of said extracellular vesicles as a medicament, and in particular, for treating various diseases.

### FIELD OF INVENTION

The present invention relates to chimeric polypeptides, nucleic acids encoding said chimeric polypeptides, and extracellular vesicles comprising said chimeric polypeptides. It further relates to the use of said extracellular vesicles as a medicament, and in particular, for treating various diseases.

### BACKGROUND OF INVENTION

Number of proteins of interest such as cellular or viral type II transmembrane proteins, proteins from the TNF or TNFR superfamily that are type II transmembrane proteins subjected to cleavage releasing their extracellular parts, proteins from the TGF-β superfamily, proteins from the CTRP family, all are known to exert effects in various human and animal conditions, including viral infection, auto-immune diseases, T-cell activation, regulation of the cellular immune response, cancer metastasis and hormonal cancers. Therefore, these proteins are promising candidates for drug development of various diseases.

Despite this promising aspect, it is sometimes difficult to find available therapies for clinical testing, because, large-scale production of certain proteins native functional forms, including post-translational modifications and proper highly multimerized complex assembly, is a daunting task. Combined with the short half-life of certain proteins, no production strategy supplying high levels of bioactive proteins at a reasonable price exists.

Moreover, all those proteins are only functional once they acquire their multimerized form, such as homodimerized, heterodimerized, trimerized, tetramerized or more. In addition, some of those proteins need to be anchored to the membrane in order to be functional. Finally, all those proteins exhibit their function at their C-terminal end, thus, their C-terminal domain needs to remain accessible either to their receptor or to their substrate. Therefore, alternative approaches providing multimeric native active forms of protein of interest are then awaited.

In the present invention, the inventors have found that certain proteins of interest can be produced in high amounts and some in native highly multimerized forms when anchored in extracellular vesicles, and that these proteins-rich extracellular vesicles can be purified and stored in characterized and qualified batches. Such standardized material opens the way for promising strategies to treat the various human physiological or pathological conditions involving said protein of interests (most of the time having therapeutic potential or being therapeutic targets), such as, for example, neurodegenerative diseases, auto-immune diseases, viral infections, T-cell immune responses, NK-cell immune responses, homeostatic imbalance associated diseases, diabetes, obesity and associated metabolic disease, insulin resistance, cardiovascular disease, inflammatory conditions, hormonal cancers and cancer progression.

### SUMMARY

The present invention relates to a chimeric polypeptide comprising, in any order:
i) an amino acid sequence of a protein of interest, wherein said protein of interest is selected from a type II transmembrane protein, a protein from the TNF or TNFR superfamily, a protein from the TGF-β superfamily, a protein from the neurotrophin family, and a protein from the CTRP family selected from CTRP1, CTRP2, CTRP3, CTRP4, CTRP5, CTRP6, CTRP7, CTRP8, CTRP9, CTRP10, CTRP11, CTRP12, CTRP13, CTRP14 and CTRP15;
ii) optionally an amino acid sequence of a transmembrane domain of a transmembrane protein; and
iii) an amino acid sequence of a pilot peptide interacting with the Endosomal Sorting Complexes Required for Transport (ESCRT) cellular machinery and/or an amino acid sequence of a sub-membrane targeting domain.

In some embodiments, the components i), ii) and iii) are organized in the chimeric polypeptide from C-terminal to N-terminal.

In some embodiments, the protein of interest is selected from neuraminidase N2, TRAIL, TNFα, CD30L, CD137L, TGF-β1, BMP7, BMP9, GDNF, BDNF, CTRP3, and OX40L.

In some embodiments, the sub-membrane targeting domain is linked to an anchoring molecule, preferably wherein the anchoring molecule is a fatty acid.

In some embodiments, the chimeric polypeptide further comprises at least one linker between the amino acid sequence of the protein of interest and the amino acid sequence of the transmembrane domain and/or between the amino acid sequence of the transmembrane domain or of the amino acid sequence of the pilot peptide and the amino acid sequence of the sub-membrane targeting domain.

In some embodiments, the transmembrane domain is selected from the transmembrane domain of a type II transmembrane protein, the transmembrane domain of influenza virus neuraminidase transmembrane protein, the transmembrane domain of CD40L, and the transmembrane domain of CD8, preferably the transmembrane domain of CD40L is as set forth in SEQ ID NO: 17 and the transmembrane domain of CD8 is as set forth in SEQ ID NO: 19, more preferably the transmembrane domain of CD40L is as set forth in SEQ ID NO: 18 and the transmembrane domain of CD8 is as set forth in SEQ ID NO: 20.

In some embodiments, the pilot peptide comprises at least one YxxL motif or DYxxL motif, and at least one PxxP motif or PPxY motif, in which “x” represents any amino acid residue, preferably in which “x” represents a proline residue.

In some embodiments, the pilot peptide comprises an amino acid sequence with SEQ ID NO: 47 or a variant thereof, wherein the variant of SEQ ID NO: 47 retains at least three YxxL and/or DYxxL motifs; and at least four PxxP motifs; wherein “x” represents any amino acid residue.

The present invention also relates to a nucleic acid encoding the chimeric polypeptide of the invention.

The present invention also relates to an extracellular vesicle comprising the chimeric polypeptide of the invention, and/or the nucleic acid of the invention, preferably wherein:
the transmembrane domain of the chimeric polypeptide is anchored in the extracellular vesicle lipid bilayer; and
the protein of interest of the chimeric polypeptide is exposed at the outer surface of the extracellular vesicle.

In some embodiments, the extracellular vesicle is an exosome, preferably having a diameter ranging from about 30 nm to about 150 nm.

The present invention also relates to a population of extracellular vesicles of the invention, optionally further comprising soluble protein of interest in its cytosol.

In some embodiments, the extracellular vesicle of the invention or the population of extracellular vesicles of the invention, are purified, preferably ultra-purified.

The present invention also relates to the nucleic acid of the invention, the extracellular vesicle of the invention or the population of extracellular vesicles of the invention, for use as a medicament.

The present invention also relates to the nucleic acid of the invention, the extracellular vesicle of the invention or the population of extracellular vesicles of the invention, for use in the treatment of a disease, disorder or condition selected from neurodegenerative diseases, auto-immune diseases, viral infections, T-cell immune responses, NK-cell immune responses, homeostatic imbalance associated diseases, diabetes, obesity and associated metabolic disease, insulin resistance, cardiovascular diseases, inflammatory conditions, hormonal cancers and cancer progression.

### Definitions

In the present invention, the following terms have the following meanings:

As used herein, the use of “a” and “an” means “at least one” or “one or more” unless the context clearly dictates otherwise. It should be noted that the term “and” or the term “or” are generally employed in their sense including “and/or” unless the context clearly dictates otherwise.

“About” preceding a figure encompasses plus or minus 10%, or less, of the value of said figure. It is to be understood that the value to which the term “about” refers is itself also specifically, and preferably, disclosed.

“CD8” refers to a transmembrane glycoprotein that serves as a co-receptor for the T cell receptor (TCR). In humans, CD8 transmembrane domain comprise an amino acid sequence with SEQ ID NO: 19.

“CD40 ligand” (also called “CD40L” or “CD154”) refers to a transmembrane protein, member of the tumor necrosis factor (TNF) superfamily. In humans, CD40L transmembrane domain comprise an amino acid sequence with SEQ ID NO: 17.

“Chimeric”, when referring to a polypeptide, refers to a polypeptide that combines several domains of at least two different types by their function and/or by their cellular localization, wherein the at least two of these domains come either from distinct proteins of the same or different species, or from the same protein of different species.

“Domain”, when referring to a protein or a polypeptide, refers to a region having a structural and/or functional property for said protein or polypeptide. As used herein, a “transmembrane domain” refers to a functional region of a protein or polypeptide that spans the phospholipid bilayer of a biological membrane.

“ESCRT” or “endosomal sorting complexes required for transport” refers originally to a cellular machinery made up of five multi-subunit protein complexes, which act cooperatively at specialized endosomes to facilitate the movement of specific cargoes from the limiting membrane into vesicles that bud into the endosome lumen. This machinery is hijacked by several envelope viruses to bud from cellular membranes, including the plasma membrane.

“Exosome” refers to an extracellular vesicle that is produced in the endosomal compartment of eukaryotic cells (Théry et al., 2018. J Extracell Vesicles. 7(1):1535750; Yáñez-Mó et al., 2015. J Extracell Vesicles. 4:27066; van Niel et al., 2018. Nat Rev Mol Cell Biol. 19(4):213-228). Typically, exosomes harbor at their surface the CD81, CD63 and CD9 markers.

“Expression vector” refers to a vector capable of directing expression of a nucleic acid sequence of interest (such as, e.g., a nucleic acid according to the present invention) in an appropriate host cell, comprising a promoter operatively linked to the nucleic acid sequence of interest, itself operatively linked to a termination sequence.

“Extracellular vesicle” refers to any vesicle composed of a lipid bilayer that is naturally released from a cell and comprises a cytosolic fraction of said cell. This expression in particular includes vesicles secreted into the extracellular space, i.e., “exosomes”.

“Identity” or “sequence identity”: refers to the number of identical or similar nucleotides or amino acid residues in a comparison between a test and a reference sequence. Sequence identity can be determined by sequence alignment of nucleic acid or amino acid sequences to identify regions of similarity or identity. For purposes herein, sequence identity is generally determined by alignment to identify identical nucleotides or amino acid residues. The alignment can be local or global. Matches, mismatches and gaps can be identified between compared sequences. Gaps are null nucleotides or amino acid residues inserted between the residues of aligned sequences so that identical or similar characters are aligned. Generally, there can be internal and terminal gaps. When using gap penalties, sequence identity can be determined with no penalty for end gaps (e.g., terminal gaps are not penalized). Alternatively, sequence identity can be determined without taking into account gaps as

$$\frac{number of identical positions}{length of the total aligned sequence} \times 100 .$$

For purposes herein, sequence identity can be determined by standard alignment algorithm programs used with default gap penalties established by each supplier. Default parameters for the GAP program can include:
a unary comparison matrix (containing a value of 1 for identities and 0 for non-identities) and the weighted comparison matrix of Gribskov & Burgess (1986. Nucleic Acids Res. 14(16):6745-63), as described by Schwartz & Dayhoff (1979. Matrices for detecting distant relationships. In Dayhoff (Ed.), Atlas of protein sequences. 5:353-358. Washington, DC: National Biomedical Research Foundation);
a penalty of 3.0 for each gap and an additional 0.10 penalty for each symbol in each gap; and
no penalty for end gaps.

“Isolated” and any declension thereof, as well as “purified” and any declension thereof, are used interchangeably, and mean that a molecular entity to which it refers (e.g., a polypeptide, a nucleic acid, an extracellular vesicle, etc.) is substantially free of other components (i.e., of contaminants) found in the natural environment in which said molecular entity is normally found. Preferably, an isolated or purified molecular entity (e.g., an isolated or purified polypeptide, an isolated or purified nucleic acid, an isolated or purified extracellular vesicle, etc.) is substantially free of other molecular entities with which it is associated in a cell. By “substantially free”, it is meant that said isolated or purified molecular entity represents more than 50% of a heterogeneous composition (i.e., is at least 50% pure), preferably, more than 60%, more than 70%, more than 80%, more than 90%, more than 95%, and more preferably still more than 98% or 99%. Purity can be evaluated by various methods known by the one skilled in the art, including, but not limited to, chromatography, gel electrophoresis, immunoassay, composition analysis, biological assay, and the like.

“Linker” or “spacer” interchangeably refer to an amino acid sequence, typically a synthetic amino acid sequence, that connects or links two peptide or polypeptide sequences together. Linkers typically connect two peptide or polypeptide sequences via peptide bonds. Linkers are well-known in the art; see., e.g., Chen et al., 2013 (Adv Drug Deliv Rev. 65(10):1357-1369) or Klein et al., 2014 (Protein Eng Des Sel. 27(10):325-330), the content of which is incorporated herein by reference. Examples of suitable linkers include so-called “GS linkers” or “Gly-Ser linkers”, i.e., amino acid sequences essentially consisting of glycine (G) and serine(S) residues, and usually—but not always-comprising two or more repeats of a peptide motif. GS linkers are well-known and widely used in the art, in particular for their flexibility properties. In some embodiments, the GS linker comprises or consists of an amino acid sequence (GₓS)_{y} or (SGₓ)_{y}, wherein x ranges from 1 to 5 or more, such as 1, 2, 3, 4, 5 or more; and y ranges from 1 to 8 or more, such as 1, 2, 3, 4, 5, 6, 7, 8 or more. In some embodiments, the GS linker with amino acid sequence (GₓS)_{y} or (SGₓ)_{y} can further comprise one or several additional G and/or S residues in N-terminal and/or in C-terminal. In some embodiments, the GS linker comprises or consists of an amino acid sequence (GS)_{y}, (GGS)_{y}, (GGGS)_{y} (SEQ ID NO: 48), (GGGGS)_{y} (SEQ ID NO: 49), or (GGGGGS)_{y} (SEQ ID NO: 50), wherein y ranges from 1 to 8 or more, such as 1, 2, 3, 4, 5, 6, 7, 8 or more. Another example of suitable linker includes so-called “glycine linkers”, i.e., amino acid sequences essentially consisting of glycine (G) residues. Glycine linkers are well-known and widely used in the art, in particular for their flexibility properties. In some embodiments, the glycine linker comprises or consists of an amino acid sequence (G) z, wherein z ranges from 1 to 10 or more, such as 1, 2, 3, 4, 5, 6, 7, 8, 9, 10 or more.

“Local alignment” refers to an alignment that aligns two sequences, but only aligns those portions of the sequences that share similarity or identity. Hence, a local alignment determines if sub-segments of one sequence are present in another sequence. If there is no similarity, no alignment will be returned. Local alignment algorithms include BLAST or Smith-Waterman algorithm (Smith & Waterman, 1981. Adv Appl Math. 2(4): 482-9). For example, 50% sequence identity based on local alignment means that in an alignment of the full sequence of two compared sequences of any length, a region of similarity or identity of 100 nucleotides or amino acid residues in length has 50% of the residues that are the same in the region of similarity or identity.

“Pilot peptide” refers to a peptide that interact with ESCRT proteins. Said pilot peptide is capable of being addressed to membrane vesicles, in particular to exosome-forming vesicles, or to the cell compartment(s) involved in the formation of membrane vesicles, and in particular of exosome-forming vesicles in eukaryotic cells.

“Selected from” is used herein according to common patent application drafting terminology, to introduce a list of elements among which one or more item(s) is (are) selected. Any occurrence of “selected from” in the specification may be replaced by “selected from the group comprising or consisting of” and reciprocally without changing the meaning thereof.

“Subject” refers to a mammal, preferably a human. In some embodiments, a subject may be a “patient”, i.e., a warm-blooded animal, more preferably a human, who/which is awaiting the receipt of, or is receiving medical care or was/is/will be the object of a medical procedure, or is monitored for the development of a disease.

“Sub-membrane targeting domain” or “membrane targeting domain” or “membrane recruitment domain” are used interchangeably to refer to a domain capable of, in a cell and in particular in a eukaryotic cell (e.g., in an exosome-producing cell), to anchor itself to a cell membrane and/or a vesicular membrane without being inserted into said membrane, said anchoring being achieved by means of one or more anchoring molecule(s) and/or by interactions (e.g., electrostatic interactions) between the sub-membrane targeting domain and the membrane. In a particular embodiment, the sub-membrane targeting domain is capable of binding to, or interacting with, the inner surface of the cell membrane (i.e., the cytoplasmic side of the cell membrane) and/or with the inner surface of vesicular membranes (i.e., the lumen side of the vesicular membrane).

“Therapeutically effective amount” refers to the level or amount of a chimeric polypeptide, nucleic acid, extracellular vesicle, population of extracellular vesicles, composition, pharmaceutical composition, medicament, etc. that is aimed at, without causing significant negative or adverse side effects to the target, (1) delaying or preventing the onset of a disease, disorder, or condition; (2) slowing down or stopping the progression, aggravation, or deterioration of one or more symptoms of the disease, disorder, or condition; (3) bringing about ameliorations of the symptoms of the disease, disorder, or condition; (4) reducing the severity or incidence of the disease, disorder, or condition; or (5) curing the disease, disorder, or condition. A therapeutically effective amount may be administered prior to the onset of the disease, disorder, or condition, for a prophylactic or preventive action. Alternatively or additionally, the therapeutically effective amount may be administered after initiation of the disease, disorder, or condition, for a therapeutic action.

“Transmembrane protein” refers to a protein which comprises at least one transmembrane domain, allowing it to be anchored in the phospholipid bilayer of a biological membrane. A transmembrane domain is generally hydrophobic-helical, and can contain several, in particular 2, 3, 4, 5, 6, 7, 8, 9 or 10, or even 20 or more, hydrophobic β-helices. It can also be arranged in a β-sheet, e.g., in a β-barrel structure typically composed of 8 to 22 β-strands. The transmembrane proteins can also be classified according to the position of the N- and C-terminal on the different sides of the lipid layers: Types I, II, III and IV. Type I transmembrane proteins are anchored to the lipid membrane with a stop-transfer anchor sequence and have their N-terminal domains targeted to the (ER) lumen during synthesis (and the extracellular space, if mature forms are located on cell membranes). Type II and III are anchored with a signal-anchor sequence, with type II being targeted to the ER lumen with its C-terminal domain, while type III have their N-terminal domains targeted to the ER lumen. Type IV is subdivided into IV-A, with their N-terminal domains targeted to the cytosol and IV-B, with an N-terminal domain targeted to the lumen.

“Treating” or “treatment” or “alleviation” refers to both therapeutic treatment and prophylactic or preventative measures; wherein the object is to prevent or slow down (lessen) the targeted pathologic condition or disorder. “Treating” or “treatment” may refer to a therapeutic treatment. “Treating” or “treatment” may refer to a prophylactic or preventive treatment. “Treating” or “treatment” may refer to both a prophylactic (or preventive) treatment and a therapeutic treatment. Those in need of treatment include those already with the disorder as well as those prone to have the disorder or those in whom the disorder is to be prevented. In some embodiments, a subject is successfully “treated” for a disease, disorder, or condition if, after receiving a therapeutic amount of a chimeric polypeptide, nucleic acid, extracellular vesicle, population of extracellular vesicles, composition, pharmaceutical composition, medicament, etc., the subject shows at least one of the following: relief to some extent of one or more of the symptoms associated with the disease, disorder, or condition to be treated; reduced morbidity and mortality; and improvement in quality of life issues. The above parameters for assessing successful treatment and improvement in the disease are readily measurable by routine procedures familiar to a physician.

“Vector” refers to a nucleic acid capable of transporting a nucleic acid of interest (such as, e.g., a nucleic acid according to the present invention) to which it has been linked. Vectors capable of directing the expression of a nucleic acid of interest (such as, e.g., a nucleic acid according to the present invention) are referred to as “expression vectors”. In general, expression vectors are in the form of plasmids. Herein, the terms “plasmid” and “vector” are used interchangeably. However, other forms of expression vectors, which serve equivalent functions, are also encompassed under the term vector.

### DETAILED DESCRIPTION

The present invention relates to a chimeric polypeptide comprising, in any order:
i) an amino acid sequence of a protein of interest, wherein said protein of interest is selected from a type II transmembrane protein, a protein from the TNF or TNFR superfamily, a protein from the TGF-β superfamily, and a protein from the CTRP family selected from CTRP1, CTRP2, CTRP3, CTRP4, CTRP5, CTRP6, CTRP7, CTRP8, CTRP9, CTRP10, CTRP11, CTRP12, CTRP13, CTRP14 and CTRP15,
ii) optionally, an amino acid sequence of a transmembrane domain of a transmembrane protein, and
iii) an amino acid sequence of a pilot peptide interacting with the Endosomal Sorting Complexes Required for Transport (ESCRT) cellular machinery and/or an amino acid sequence of a sub-membrane targeting domain.

In some embodiments, components (i), (ii), and (iii) are organized in the chimeric polypeptide from N-terminal to C-terminal or from C-terminal to N-terminal. In some embodiments, components (i), (ii), and (iii) are organized in the chimeric polypeptide from C-terminal to N-terminal.

According to the invention, the protein of interest is a therapeutic protein selected from a type II transmembrane protein, a protein from the TNF or TNFR superfamily, a protein from the TGF-β superfamily, and a protein from the CTRP family selected from CTRP1, CTRP2, CTRP3, CTRP4, CTRP5, CTRP6, CTRP7, CTRP8, CTRP9, CTRP10, CTRP11, CTRP12, CTRP13, CTRP14 and CTRP15.

Type II transmembrane proteins are single-pass transmembrane proteins with their N-terminus on the cytoplasmic side of the membrane. They are anchored to the lipid membrane by a signal-anchoring sequence and target the endoplasmic reticulum lumen with their C-terminal domain.

In some embodiments, the protein of interest is a type II transmembrane protein.

Examples of type II transmembrane proteins include, but are not limited to, neuraminidase N2 from influenza virus, BRI2 (also known as Integral Membrane Protein 2B or ITM2B), proteins from the type II transmembrane serine protease (TTSP) family, Golgi phosphoprotein 2 (GOLPH2, also known as Golgi membrane protein 1, GOLM1, or Golgi membrane protein GP73), neprilysin (also known as neutral endopeptidase-24.11, CD10, SFE, EPN, SCA43, CMT2T, or NEP), proteins from the asialoglycoprotein receptors (ASGPR) family, proteins from the Dectin C-type lectin-like receptors (CTLRs) family (such as killer-cell lectin-like receptors (KLRs) that form homo-NKG2D or hetero-dimers NKG2A).

In some embodiments, the protein of interest is neuraminidase N2 from influenza virus of type A H3N2 strain as set forth in SEQ ID NO: 1, or a variant thereof.

Hence, in some embodiments, the protein of interest comprises or consist of an amino acid sequence with SEQ ID NO: 1, or a variant thereof. In some embodiments, a variant of the amino acid sequence with SEQ ID NO: 1 comprises an amino acid sequence sharing at least 70% of global sequence identity, preferably at least 75%, 80%, 85%, 90%, 95%, 96%, 97%, 98%, 99% or more of global sequence identity with the amino acid sequence of SEQ ID NO: 1.

Additionally, or alternatively, a variant of the amino acid sequence with SEQ ID NO: 1 comprises an amino acid sequence sharing at least 70% of local sequence identity, preferably at least 75%, 80%, 85%, 90%, 95%, 96%, 97%, 98%, 99% or more of local sequence identity with the amino acid sequence of SEQ ID NO: 1.

Proteins of the Tumor Necrosis factor (TNF) and TNF receptor (TNFR) superfamilies are type II transmembrane proteins containing TNF homology domain and forming trimers. Members of these superfamilies exist either i) anchored to the plasma membrane in a similar fashion than classical type II transmembrane proteins, or ii) can be released from the cell membrane by extracellular proteolytic cleavage and function as a cytokine. The TNF family contains 19 members, while the TNFR family contains 27 members.

Examples of proteins from the TNF superfamily include, but are not limited to, Lymphotoxin alpha (LTα, also known as TNFβ, or TNFSF1B), Tumor Necrosis Factor (TNF also known as TNFα, Dif, Necrosin, or TNFSF1A), Lymphotoxin beta (LTβ, also known as TNFSF3, or TNFγ), OX40 ligand (OX40L, also known as TNFSF4, CD252, Gp34, or CD134L), CD40 ligand (CD40L, also known as TNFSF5, CD154, TRAP, Gp39, or T-BAM), Fas ligand (FasL, also known as TNFSF6, CD178, APTL, or CD95L), CD27 ligand (CD27L, also known as TNFSF7, or CD70), CD30 ligand (CD30L, also known as TNFSF8, or CD153), CD137 ligand (CD137L, also known as TNFSF9, 4-1BB ligand, or 4-1BBL), TNF-related apoptosis-inducing ligand (TRAIL, also known as TNFSF10, CD253, or APO-2L), Receptor activator of nuclear factor kappa-B ligand (RANKL, also known as TNFSF11, CD254, OPGL, TRANCE, or ODF), TNF-related weak inducer of apoptosis (TWEAK, also known as TNFSF12, APO-3L, or DR3L), A proliferation-inducing ligand (APRIL, also known as TNFSF13, CD256, TALL-2, or TRDL1), B-cell activating factor (BAFF, also known as TNFSF13B, CD257, BLyS, TALL-1, or TNFSF20), homologous to lymphotoxin, exhibits inducible expression and competes with HSV glycoprotein D for binding to herpesvirus entry mediator, a receptor expressed on T lymphocytes (LIGHT, also known as TNFSF14, CD258, or HVEML), Vascular endothelial growth inhibitor (VEGI, also known as TNFSF15, TL1, or TL-1A), TNF superfamily member 18 (TNFSF18, also known as GITRL, AITRL, or TL-6) and ectodysplasin A (EDA, also known as ED1-A1, or ED1-A2).

Examples of proteins from the TNF receptor superfamily include, but are not limited to, Tumor necrosis factor receptor 1 (TNFR1, also known as TNFRSF1A, or CD120a), Tumor necrosis factor receptor 2 (TNFR2, also known as TNFRSF1B, or CD120b), Lymphotoxin beta receptor (LTBR, also known as TNFRSF3, LTBR, or CD18), OX40 (also known as TNFRSF4, or CD134), CD40 (also known as TNFRSF5, or Bp50), Fas receptor (also known as TNFRSF6, Apo-1, or CD95), Decoy receptor 3 (DCR3, also known as TNFRSF6B, TR6, or M68), CD27 (also known as TNFRSF7, S152, or Tp55), CD30 (also known as TNFRSF8, Ki-1, or TNR8), 4-1BB (also known as TNFRSF9, or CD137), Death receptor 4 (DR4, also known as TNFRSF10A, TRAILR1, Apo-2, or CD261), Death receptor 5 (DR5, also known as TNFRSF10B, TRAILR2, or CD262), Decoy receptor 1 (DCR1, also known as TNFRSF10C, TRAILR3, LIT, TRID, or CD263), Decoy receptor 2 (DCR2, also known as TNFRSF10D, TRAILR4, TRUNDD, or CD264), RANK (also known as TNFRSF11A, or CD265), Osteoprotegerin (OPG, also known as TNFRSF11B, OCIF, or TR1), TWEAK receptor (also known as TNFRSF12A, Fn14, or CD266), Transmembrane activator and CAML interactor (TACI, also known as TNFRSF13B, IGAD2, or CD267), BAFF receptor (also known as TNFRSF13B, or CD268), Herpes virus entry mediator (HVEM, also known as TNFRSF14, ATAR, TR2, or CD270), Nerve growth factor receptor (NGFR, also known as TNFRSF16, p75NTR, or CD271), B-cell maturation antigen (BCMA, also known as BCM, TNFRSF17, CD269, or TNFRSF13A), Glucocorticoid-induced TNFR-related (GITR, also known as TNFRSF18, AITR, or CD357), TROY (also known as TNFRSF19, TAJ, or TRADE), Death receptor 6 (DR6, also known as TNFRSF21, or CD358), Death receptor 3 (DR3, also known as TNFRSF25, Apo-3, TRAMP, LARD, or WS-1), and Ectodysplasin A2 receptor (EDA2R also known as TNFRSF27, or XEDAR).

In some embodiments, the protein of interest is a protein from the TNF or TNFR superfamily. In some embodiments, the protein of interest is selected from LTα, TNFα, LTβ, OX40L, CD40L, FasL, CD27L, CD30L, CD137L, TRAIL, RANKL, TWEAK, APRIL, BAFF, LIGHT, VEGI, TNFSF18, EDA, TNFR1, TNFR2, LTβR, OX40, CD40, Fas receptor, DCR3, CD27, CD30, 4-1BB, DR4, DR5, DCR1, DCR2, RANK, OPG, TWEAK receptor, TACI, BAFF receptor, HVEM, NGFR, BCMA, GITR, TROY, DR6, DR3, and EDA2R.

In some embodiments, the protein of interest is a protein from the TNF superfamily. In some embodiments, the protein of interest is selected from LTα, TNFα, LTβ, OX40L, CD40L, FasL, CD27L, CD30L, CD137L, TRAIL, RANKL, TWEAK, APRIL, BAFF, LIGHT, VEGI, TNFSF18, and EDA.

In some embodiments, the protein of interest is a protein from the TNFR superfamily. In some embodiments, the protein of interest is selected from TNFR1, TNFR2, LTβR, OX40, CD40, Fas receptor, DCR3, CD27, CD30, 4-1BB, DR4, DR5, DCR1, DCR2, RANK, OPG, TWEAK receptor, TACI, BAFF receptor, HVEM, NGFR, BCMA, GITR, TROY, DR6, DR3, and EDA2R.

In some embodiments, the protein of interest is TRAIL as set forth in SEQ ID NO: 2, or a variant thereof. In some embodiments, the protein of interest is TRAIL external domain as set forth in SEQ ID NO: 3, or a variant thereof. In some embodiments, the protein of interest is TRAIL TNF-like domain as set forth in SEQ ID NO: 4, or a variant thereof.

Hence, in some embodiments, the protein of interest comprises or consist of an amino acid sequence with SEQ ID NO: 2, 3, or 4, or a variant thereof. In some embodiments, a variant of the amino acid sequence with SEQ ID NO: 2, 3, or 4 comprises an amino acid sequence sharing at least 70% of global sequence identity, preferably at least 75%, 80%, 85%, 90%, 95%, 96%, 97%, 98%, 99% or more of global sequence identity with the amino acid sequence of SEQ ID NO: 2, 3, or 4.

Additionally or alternatively, a variant of the amino acid sequence with SEQ ID NO: 2, 3, or 4 comprises an amino acid sequence sharing at least 70% of local sequence identity, preferably at least 75%, 80%, 85%, 90%, 95%, 96%, 97%, 98%, 99% or more of local sequence identity with the amino acid sequence of SEQ ID NO: 2, 3, or 4.

In some embodiments, the protein of interest is TNFα as set forth in SEQ ID NO: 5, or a variant thereof. In some embodiments, the protein of interest is TNFα external domain as set forth in SEQ ID NO: 6, or a variant thereof. In some embodiments, the protein of interest is TNFα mature domain as set forth in SEQ ID NO: 7, or a variant thereof.

Hence, in some embodiments, the protein of interest comprises or consist of an amino acid sequence with SEQ ID NO: 5, 6, or 7, or a variant thereof. In some embodiments, a variant of the amino acid sequence with SEQ ID NO: 5, 6, or 7 comprises an amino acid sequence sharing at least 70% of global sequence identity, preferably at least 75%, 80%, 85%, 90%, 95%, 96%, 97%, 98%, 99% or more of global sequence identity with the amino acid sequence of SEQ ID NO: 5, 6, or 7.

Additionally or alternatively, a variant of the amino acid sequence with SEQ ID NO: 5, 6, or 7 comprises an amino acid sequence sharing at least 70% of local sequence identity, preferably at least 75%, 80%, 85%, 90%, 95%, 96%, 97%, 98%, 99% or more of local sequence identity with the amino acid sequence of SEQ ID NO: 5, 6, or 7.

In some embodiments, the protein of interest is CD30L as set forth in SEQ ID NO: 8, or a variant thereof. In some embodiments, the protein of interest is CD30L external domain as set forth in SEQ ID NO: 9, or a variant thereof.

Hence, in some embodiments, the protein of interest comprises or consist of an amino acid sequence with SEQ ID NO: 8, or 9, or a variant thereof. In some embodiments, a variant of the amino acid sequence with SEQ ID NO: 8, or 9 comprises an amino acid sequence sharing at least 70% of global sequence identity, preferably at least 75%, 80%, 85%, 90%, 95%, 96%, 97%, 98%, 99% or more of global sequence identity with the amino acid sequence of SEQ ID NO: 8, or 9.

Additionally or alternatively, a variant of the amino acid sequence with SEQ ID NO: 8, or 9 comprises an amino acid sequence sharing at least 70% of local sequence identity, preferably at least 75%, 80%, 85%, 90%, 95%, 96%, 97%, 98%, 99% or more of local sequence identity with the amino acid sequence of SEQ ID NO: 8, or 9.

In some embodiments, the protein of interest is CD137L as set forth in SEQ ID NO: 10, or a variant thereof.

Hence, in some embodiments, the protein of interest comprises or consist of an amino acid sequence with SEQ ID NO: 10, or a variant thereof. In some embodiments, a variant of the amino acid sequence with SEQ ID NO: 10 comprises an amino acid sequence sharing at least 70% of global sequence identity, preferably at least 75%, 80%, 85%, 90%, 95%, 96%, 97%, 98%, 99% or more of global sequence identity with the amino acid sequence of SEQ ID NO: 10.

Additionally or alternatively, a variant of the amino acid sequence with SEQ ID NO: 10 comprises an amino acid sequence sharing at least 70% of local sequence identity, preferably at least 75%, 80%, 85%, 90%, 95%, 96%, 97%, 98%, 99% or more of local sequence identity with the amino acid sequence of SEQ ID NO: 10.

In some embodiments, the protein of interest is OX40L as set forth in SEQ ID NO: 90, or a variant thereof.

Hence, in some embodiments, the protein of interest comprises or consist of an amino acid sequence with SEQ ID NO: 90, or a variant thereof. In some embodiments, a variant of the amino acid sequence with SEQ ID NO: 90 comprises an amino acid sequence sharing at least 70% of global sequence identity, preferably at least 75%, 80%, 85%, 90%, 95%, 96%, 97%, 98%, 99% or more of global sequence identity with the amino acid sequence of SEQ ID NO: 90.

Additionally or alternatively, a variant of the amino acid sequence with SEQ ID NO: 90 comprises an amino acid sequence sharing at least 70% of local sequence identity, preferably at least 75%, 80%, 85%, 90%, 95%, 96%, 97%, 98%, 99% or more of local sequence identity with the amino acid sequence of SEQ ID NO: 90.

Proteins of the Transforming growth factor beta (TGF-β) superfamily are synthetized in the form of precursors in which the active protein is located at the C-terminal end of the polypeptide and which is associated to a propeptide located at the N-terminal end of the polypeptide. Those proteins require proteolytic cleavage to exhibit biological activity. Proteins of the TGF-β superfamily are only active as homo- or heterodimer, wherein the two chains are linked by a single interchain disulfide bond. The TGF-β superfamily is subdivided in subfamilies: the TGF-β subfamily, the bone morphogenetic proteins, the growth differentiation factors, the activin and inhibin subfamily, the left-right determination factors, the glial cell line-derived neurotrophic factor (GDNF) subfamily, and a group encompassing various divergent members.

Examples of proteins from the TGF-β superfamily include, but are not limited to, TGF-β1 (also known as CED, DPD1, or LAP), TGF-β2 (also known as LDS4, or G-TSF), TGF-β3 (also known as ARVD, ARVD1, RNHF, or LDS5), Bone morphogenetic protein 2 (BMP2, also known as SSFSC, or SSFSC1), BMP3 (also known as osteogenin), BMP4 (also known as MCOPS6, OFC11, or ZYME), BMP5, BMP6 (also known as VGR, or VGR1), BMP7 (also known as OP-1), BMP8a, BMP8b (also known as OP2), BMP10, BMP11 (also known as GDF11, or VHO), BMP15 (also known as GDF9B, ODG2, or POF4), Growth differentiation factor 1 (GDF1, also known as DORV, DTGA3, RAI, CHTD6, UOG1, LASS1, CERS1, or LAG1), GDF2 (also known as BMP9, or HHT5), GDF3 (also known as KFS3, MCOP7, MCOPCB6, Vg-related gene 2, or Vgr-2), GDF5 (also known as BDA1C, BMP14, CDMP1, LAP-4, LAP4, OS5, SYM1B, SYNS2, or DUPANS), GDF6 (also known as BMP13, CDMP2, KFM, KFS, KFS1, KFSL, LCA17, MCOP4, MCOPCB6, SCDO4, SGM1, or SYNS4), GDF8 (also known as myostatin, or MSTN), GDF9 (also known as PF14), GDF10 (also known as BMP3B, or BIP), GDF15 (also known as MIC-1, MIC1, NAG-1, PDF, PLAB, PTGFB, or TGF-PL), Activin A, Activin B, Activin C, Activin D, Inhibin A, Inhibin B, left-right determination factor 1 (lefty-1), lefty-2, nodal (also known as HTX5), Artemin (ARTN, also known as enovin, EVN, neublastin, or NBN), Neurturin (NRTN), Persephin (PSPN), Glial cell line-derived neurotrophic factor (GDNF), and Anti-Müllerian hormone (AMH, also known as Müllerian-inhibiting hormone, MIH, MIS, or antim).

In some embodiments, the protein of interest is a protein from the TGF-β superfamily. In some embodiments, the protein of interest is selected from TGF-β1, TGF-β2, TGF-β3, BMP2, BMP3, BMP4, BMP5, BMP6, BMP7, BMP8a, BMP8b, BMP9, BMP10, BMP11, BMP15, GDF1, GDF2, GDF3, GDF5, GDF6, GDF8, GDF9, GDF10, GDF11, GDF15, Activin A, Activin B, Activin C, Activin D, Inhibin A, Inhibin B, lefty-1, lefty-2, ARTN, GDNF, NRTN, PSPN, and AMH.

Examples of proteins from the TGF-β subfamily include, but are not limited to, TGF-β1, TGF-β2, and TGF-β3. In some embodiments, the protein of interest is selected from TGF-β1, TGF-β2, and TGF-β3.

Examples of proteins from the bone morphogenetic proteins subfamily include but are not limited to, BMP2, BMP3, BMP4, BMP5, BMP6, BMP7, BMP8a, BMP8b, BMP9, BMP10, BMP11, and BMP15. In some embodiments, the protein of interest is selected from BMP2, BMP3, BMP4, BMP5, BMP6, BMP7, BMP8a, BMP8b, BMP9, BMP10, BMP11, and BMP15.

Examples of proteins from the growth differentiation factors subfamily include, but are not limited to, GDF1, GDF2, GDF3, GDF5, GDF6, GDF8, GDF9, GDF10, GDF11, and GDF15. In some embodiments, the protein of interest is selected from GDF1, GDF2, GDF3, GDF5, GDF6, GDF8, GDF9, GDF10, GDF11, and GDF15.

Examples of proteins from the activin and inhibin subfamily include, but are not limited to, Activin A, Activin B, Activin C, Activin D, Inhibin A, and Inhibin B. In some embodiments, the protein of interest is selected from Activin A, Activin B, Activin C, Activin D, Inhibin A, and Inhibin B.

Examples of proteins from the left-right determination factors subfamily include, but are not limited to, lefty-1 and lefty-2. In some embodiments, the protein of interest is selected from lefty-1 and lefty-2.

Examples of proteins from the GDNF subfamily include, but are not limited to, ARTN, GDNF, NRTN, and PSPN. In some embodiments, the protein of interest is selected from ARTN, GDNF, NRTN, and PSPN.

In some embodiments, the protein of interest is TGF-β1 as set forth in SEQ ID NO: 11, or a variant thereof.

Hence, in some embodiments, the protein of interest comprises or consist of an amino acid sequence with SEQ ID NO: 11, or a variant thereof. In some embodiments, a variant of the amino acid sequence with SEQ ID NO: 11 comprises an amino acid sequence sharing at least 70% of global sequence identity, preferably at least 75%, 80%, 85%, 90%, 95%, 96%, 97%, 98%, 99% or more of global sequence identity with the amino acid sequence of SEQ ID NO: 11.

Additionally or alternatively, a variant of the amino acid sequence with SEQ ID NO: 11 comprises an amino acid sequence sharing at least 70% of local sequence identity, preferably at least 75%, 80%, 85%, 90%, 95%, 96%, 97%, 98%, 99% or more of local sequence identity with the amino acid sequence of SEQ ID NO: 11.

In some embodiments, the protein of interest is BMP7 as set forth in SEQ ID NO: 12, or a variant thereof.

Hence, in some embodiments, the protein of interest comprises or consist of an amino acid sequence with SEQ ID NO: 12, or a variant thereof. In some embodiments, a variant of the amino acid sequence with SEQ ID NO: 12 comprises an amino acid sequence sharing at least 70% of global sequence identity, preferably at least 75%, 80%, 85%, 90%, 95%, 96%, 97%, 98%, 99% or more of global sequence identity with the amino acid sequence of SEQ ID NO: 12.

Additionally or alternatively, a variant of the amino acid sequence with SEQ ID NO: 12 comprises an amino acid sequence sharing at least 70% of local sequence identity, preferably at least 75%, 80%, 85%, 90%, 95%, 96%, 97%, 98%, 99% or more of local sequence identity with the amino acid sequence of SEQ ID NO: 12.

In some embodiments, the protein of interest is BMP9 as set forth in SEQ ID NO: 13, or a variant thereof.

Hence, in some embodiments, the protein of interest comprises or consist of an amino acid sequence with SEQ ID NO: 13, or a variant thereof. In some embodiments, a variant of the amino acid sequence with SEQ ID NO: 13 comprises an amino acid sequence sharing at least 70% of global sequence identity, preferably at least 75%, 80%, 85%, 90%, 95%, 96%, 97%, 98%, 99% or more of global sequence identity with the amino acid sequence of SEQ ID NO: 13.

Additionally or alternatively, a variant of the amino acid sequence with SEQ ID NO: 13 comprises an amino acid sequence sharing at least 70% of local sequence identity, preferably at least 75%, 80%, 85%, 90%, 95%, 96%, 97%, 98%, 99% or more of local sequence identity with the amino acid sequence of SEQ ID NO: 13.

In some embodiments, the protein of interest is GDNF as set forth in SEQ ID NO: 14, or a variant thereof.

Hence, in some embodiments, the protein of interest comprises or consist of an amino acid sequence with SEQ ID NO: 14, or a variant thereof. In some embodiments, a variant of the amino acid sequence with SEQ ID NO: 14 comprises an amino acid sequence sharing at least 70% of global sequence identity, preferably at least 75%, 80%, 85%, 90%, 95%, 96%, 97%, 98%, 99% or more of global sequence identity with the amino acid sequence of SEQ ID NO: 14.

Additionally or alternatively, a variant of the amino acid sequence with SEQ ID NO: 14 comprises an amino acid sequence sharing at least 70% of local sequence identity, preferably at least 75%, 80%, 85%, 90%, 95%, 96%, 97%, 98%, 99% or more of local sequence identity with the amino acid sequence of SEQ ID NO: 14.

Neurotrophins are growth factors that promote the survival, development and function of neurons. Neurotrophins are synthetized in the form of precursors in which the active protein is located at the C-terminal end of the polypeptide and which is associated to a propeptide located at the N-terminal end of the polypeptide. Neurotrophins require proteolytic cleavage to exhibit biological activity.

Examples of proteins from the neurotrophin family include, but are not limited to, nerve growth factor (NGF also known as NGFB, or NGF-β), Brain-derived neurotrophic factor (BDNF), Neurotrophin-3 (NT-3), and Neurotrophin-4 (NT-4). In some embodiments, the protein of interest is selected from NGF, BDNF, NT-3, and NT-4.

In some embodiments, the protein of interest is BDNF as set forth in SEQ ID NO: 15, or a variant thereof.

Hence, in some embodiments, the protein of interest comprises or consist of an amino acid sequence with SEQ ID NO: 15, or a variant thereof. In some embodiments, a variant of the amino acid sequence with SEQ ID NO: 15 comprises an amino acid sequence sharing at least 70% of global sequence identity, preferably at least 75%, 80%, 85%, 90%, 95%, 96%, 97%, 98%, 99% or more of global sequence identity with the amino acid sequence of SEQ ID NO: 15.

Additionally or alternatively, a variant of the amino acid sequence with SEQ ID NO: 15 comprises an amino acid sequence sharing at least 70% of local sequence identity, preferably at least 75%, 80%, 85%, 90%, 95%, 96%, 97%, 98%, 99% or more of local sequence identity with the amino acid sequence of SEQ ID NO: 15.

CTRP (Clq/TNF-related proteins) proteins constitute a conserved, structurally related family of 15 members, named CTRP1 to CTRP15. Every CTRP protein contains a signal peptide, a N-terminal variable domain with one or more conserved cysteine residues, a collagen-like domain (Col-Gly-X-Y), and a C-terminal globular Clq/TNF domain. All CTRP proteins can form homotrimers, and can also form high-order multimeric 3D structures composed of multiple trimers.

In some embodiments, the protein of interest is a protein from the CTRP family. In some embodiments, the protein of interest is a protein from the CTRP family with the proviso that it is not adiponectin.

In some embodiments, the protein of interest is a protein from the CTRP family selected from CTRP1, CTRP2, CTRP3, CTRP4, CTRP5, CTRP6, CTRP7, CTRP8, CTRP9, CTRP10, CTRP11, CTRP12, CTRP13, CTRP14 and CTRP15. In some embodiments, the protein of interest is selected from CTRP1, CTRP2, CTRP3, CTRP4, CTRP5, CTRP6, CTRP7, CTRP8, CTRP9, CTRP10, CTRP11, CTRP12, CTRP13, CTRP14 and CTRP15.

In some embodiments, the protein of interest is not adiponectin.

In some embodiments, the protein of interest is CTRP3 as set forth in SEQ ID NO: 16, or a variant thereof.

Hence, in some embodiments, the protein of interest comprises or consist of an amino acid sequence with SEQ ID NO: 16, or a variant thereof. In some embodiments, a variant of the amino acid sequence with SEQ ID NO: 16 comprises an amino acid sequence sharing at least 70% of global sequence identity, preferably at least 75%, 80%, 85%, 90%, 95%, 96%, 97%, 98%, 99% or more of global sequence identity with the amino acid sequence of SEQ ID NO: 16.

Additionally or alternatively, a variant of the amino acid sequence with SEQ ID NO: 16 comprises an amino acid sequence sharing at least 70% of local sequence identity, preferably at least 75%, 80%, 85%, 90%, 95%, 96%, 97%, 98%, 99% or more of local sequence identity with the amino acid sequence of SEQ ID NO: 16.

In some embodiments, the protein of interest is:
(a) a type II transmembrane protein selected from Neuraminidase N2,
(b) a protein from the TNF or TNFR superfamily selected from LTα, TNFα, LTβ, OX40L, CD40L, FasL, CD27L, CD30L, CD137L, TRAIL, RANKL, TWEAK, APRIL, BAFF, LIGHT, VEGI, TNFSF18, EDA, TNFR1, TNFR2, LTβR, OX40, CD40, Fas receptor, DCR3, CD27, CD30, 4-1BB, DR4, DR5, DCR1, DCR2, RANK, OPG, TWEAK receptor, TACI, BAFF receptor, HVEM, NGFR, BCMA, GITR, TROY, DR6, DR3, and EDA2R;
(c) a protein from the TGF-β superfamily selected from TGF-β1, TGF-β2, TGF-3, BMP2, BMP3, BMP4, BMP5, BMP6, BMP7, BMP8a, BMP8b, BMP9, BMP10, BMP11, BMP15, GDF1, GDF2, GDF3, GDF5, GDF6, GDF8, GDF9, GDF10, GDF11, GDF15, Activin A, Activin B, Activin C, Activin D, Inhibin A, Inhibin B, lefty-1, lefty-2, ARTN, GDNF, NRTN, PSPN, and AMH;
(d) a protein from the neurotrophin family selected from NGF, BDNF, NT-3, and NT-4; or
(e) a protein from the CTRP family selected from CTRP1, CTRP2, CTRP3, CTRP4, CTRP5, CTRP6, CTRP7, CTRP8, CTRP9, CTRP10, CTRP11, CTRP12, CTRP13, CTRP14 and CTRP15.

In some embodiments, the protein of interest is selected from neuraminidase N2, TRAIL, TNFα, CD30L, CD137L, TGF-β1, BMP7, BMP9, GDNF, BDNF, and CTRP3.

In some embodiments, the protein of interest is selected from neuraminidase N2, TRAIL, TNFα, CD30L, CD137L, TGF-β1, BMP7, BMP9, GDNF, BDNF, CTRP3, and OX40L.

According to the invention, the chimeric polypeptide comprises an amino acid sequence of a transmembrane domain of a transmembrane protein.

In some embodiments, the transmembrane domain is that of a transmembrane protein from any organisms, including mammals, viruses and bacteria.

In some embodiments, the transmembrane domain is that of a transmembrane protein selected from the group comprising or consisting of human proteins, non-human animal proteins, pathogenic organism or agent proteins (in particular viral proteins, bacterial proteins, parasite proteins), or tumor cell proteins.

In some embodiments, the transmembrane domain is that of a transmembrane protein of type I. Said transmembrane domain of a transmembrane protein of type I may be used in a chimeric polypeptide as described hereinabove, wherein the components i), ii), and iii), when present, are organized in the chimeric polypeptide from N-terminal to C-terminal (such as construct 1 of FIG. 2, 3 or 4).

In some embodiments, the transmembrane domain is that of a transmembrane glycoprotein of a retrovirus selected from the group comprising or consisting of bovine leukemia virus (BLV), human immunodeficiency virus (HIV) (such as, without limitation, HIV-1 or HIV-2), human T-cell leukemia virus (HTLV) (such as, without limitation, HTLV-1 or HTLV-2), and Mason-Pfizer monkey virus (MPMV).

In some embodiments, the transmembrane domain is that of Influenza virus' hemagglutinin transmembrane protein.

In some embodiments, the transmembrane domain is that of CD8.

In some embodiments, the transmembrane domain is that of a transmembrane protein of type II. Said transmembrane domain of a transmembrane protein of type II may be used in a chimeric polypeptide as described hereinabove, wherein the components i), ii), and iii), when present, are organized in the chimeric polypeptide from C-terminal to N-terminal (such as constructs 2, 3, 4, 5 of FIG. 2, 3 or 4).

In some embodiments, the transmembrane domain is that of Influenza virus' neuraminidase transmembrane protein.

In some embodiments, the transmembrane domain is that of CD40 ligand (CD40L).

In some embodiments, the transmembrane domain is that of CD40 ligand (CD40L) or CD8.

In some embodiments, the transmembrane domain of CD40L comprises or consists of an amino acid sequence with SEQ ID NO: 17, or a variant thereof. In some embodiments, the transmembrane domain comprises or consists of an amino acid sequence with SEQ ID NO: 18, or a variant thereof.

In some embodiments, the transmembrane domain of CD8 comprises or consists of an amino acid sequence with SEQ ID NO: 19, or a variant thereof. In some embodiments, the transmembrane domain comprises or consists of an amino acid sequence with SEQ ID NO: 20, or a variant thereof.

In some embodiments, a variant of the amino acid sequence with SEQ ID NO: 17, 18, 19 or 20 comprises an amino acid sequence sharing at least 70% of global sequence identity, preferably at least 75%, 80%, 85%, 90%, 95%, 96%, 97%, 98%, 99% or more of global sequence identity with the amino acid sequence of SEQ ID NO: 17, 18, 19 or 20.

Additionally or alternatively, a variant of the amino acid sequence with SEQ ID NO: 17, 18, 19 or 20 comprises an amino acid sequence sharing at least 70% of local sequence identity, preferably at least 75%, 80%, 85%, 90%, 95%, 96%, 97%, 98%, 99% or more of local sequence identity with the amino acid sequence of SEQ ID NO: 17, 18, 19 or 20.

According to the invention, the chimeric polypeptide comprises an amino acid sequence of a peptide interacting with the Endosomal Sorting Complexes Required for Transport (ESCRT) cellular machinery, otherwise known as “Pilot Peptide”.

In some embodiments, said pilot peptide is capable of being addressed to the membrane vesicles, in particular to the exosome-forming vesicles, or to the cell compartment(s) involved in the formation of the membrane vesicles, and in particular the exosome-forming vesicles in eukaryotic cells.

When integrated into a chimeric polypeptide, such as the chimeric polypeptide of the invention, the pilot peptide enables the addressing of said chimeric polypeptide to the membrane vesicles and/or to their location(s) of formation, and in particular enables the addressing of said chimeric polypeptide to the membrane of membrane vesicles, such that said polypeptide can be secreted by a cell in association with the membrane vesicles (in particular exosomes).

Pilot peptides which interact with ESCRT proteins have been described in granted patents EP 2 268 816 B1, and U.S. Pat. No. 9,546,371 B2, the relevant content of which is incorporated herein by reference.

In some embodiments, the pilot peptide comprises at least one YxxL motif, in which “x” represents any amino acid residue. In particular, it may comprise one, two or three YxxL motifs. The YxxL motif or one of the YxxL motifs of the pilot peptide may, for example, be YINL (SEQ ID NO: 21) or YSHL (SEQ ID NO: 22).

Alternatively or additionally, the pilot peptide may comprise at least one motif equivalent to a YxxL motif, for example, a YxxF motif, in which “x” represents any amino acid residue. The YxxF motif or one of the YxxF motifs of the pilot peptide may then be, for example, be YINF (SEQ ID NO: 23) or YSHF (SEQ ID NO: 24).

In some embodiments, the pilot peptide comprises a DYxxL motif, in which “x” represents any amino acid residue. The DYxxL motif or one of the DYxxL motifs of the pilot peptide may, for example, be DYINL (SEQ ID NO: 25).

Alternatively or additionally, the pilot peptide may comprise at least one motif equivalent to a DYxxL motif, for example, a DYxxF motif, in which “x” represents any amino acid residue. The DYxxF motif or one of the DYxxF motifs of the pilot peptide may then be, for example, be DYINF (SEQ ID NO: 26).

In some embodiments, the pilot peptide further comprises at least one PxxP motif, in which “x” represents any amino acid residue. In particular, it may comprise one, two, three or four PxxP motifs.

In some embodiments, the pilot peptide comprises at least one proline rich motif.

“Proline-rich motif” refers to an amino acid sequence, typically a synthetic amino acid sequence, that contains several proline (P) residues. Examples of proline rich motifs include, but are not limited to, PP, PPP, PPPP (SEQ ID NO: 89).

In some embodiments, the pilot peptide comprises at least one PP motif, at least one PPP motif, or at least one PPPP (SEQ ID NO: 89) motif.

In some embodiments, the pilot peptide comprises at least one PPxY motif, in which “x” represents any amino acid residue, preferably in which “x” represents a proline (P) residue.

In some embodiments, the PxxP motif or one of the PxxP motifs of the pilot peptide may, for example, be PSAP (SEQ ID NO: 27) or PTAP (SEQ ID NO: 28).

In some embodiments, the pilot peptide comprises at least one YxxL motif or DYxxL motif, and at least one PxxP motif.

In some embodiments, the pilot peptide comprises or consists of an amino acid sequence having one, two or three YxxL and/or DYxxL motif(s); and one, two, three or four PxxP motif(s).

In some embodiments, the pilot peptide comprises or consists of an amino acid sequence having three YxxL and/or DYxxL motifs, and four PxxP motifs.

In some embodiments, the YxxL motif or at least one of the YxxL motifs, when more than one, is located downstream, i.e., in a C-terminal position, with respect to the one or more PxxP motif(s).

Proteins having a pilot peptide comprising at least one YxxL motif include cellular proteins and viral proteins. In particular, these viral proteins are proteins of enveloped viruses, such as transmembrane glycoproteins of enveloped viruses, or herpesvirus proteins, e.g., the LMP2-A protein of the Epstein-Barr virus which comprises at least two YxxL motifs.

In some embodiments, the pilot peptide is that of a transmembrane glycoprotein of a retrovirus. In some embodiments, the pilot peptide may be that of a transmembrane glycoprotein of a retrovirus selected from the group comprising or consisting of bovine leukemia virus (BLV), human immunodeficiency virus (HIV) (such as, without limitation, HIV-1 or HIV-2), human T-cell leukemia virus (HTLV) (such as, without limitation, HTLV-1 or HTLV-2), and Mason-Pfizer monkey virus (MPMV).

In some embodiments, the pilot peptide comprises one of the following amino acid sequences:

(SEQ ID NO: 29)
PxxPxxxxPxxPxSxYxxLxPxxPExYxxLxPxxPDYxxL;
(SEQ ID NO: 30)
PxxPxₙPxxPxₙSxYxxLxₙPxxPEXₙYxxLxₙPxxPDYxxL;
(SEQ ID NO: 31)
PxxPxxxxPxxPxSxYxxLxPxxPExYxxLxPxxPDYxxLxxxx;
and
(SEQ ID NO: 32)
PxxPxₙPxxPxNSxYxxLxₙPxxPEXₙYxxLxₙPxxPDYxxLxxxx;

in which “x” and “xₙ”, respectively, represent any amino acid residue and any one or several amino acid residue(s).

In some embodiments, the pilot peptide comprises one of the following amino acid sequences:

(SEQ ID NO: 33)
PxxPxxxxxxxxxxxxYxxL;
(SEQ ID NO: 34)
PxxPxxxxxxxxxxxDYxxL;
(SEQ ID NO: 35)
PxxPxxYxxxxxxxxxYxxL;
(SEQ ID NO: 36)
PxxPxxYxxxxxxxxDYxxL;
(SEQ ID NO: 37)
PxxPExYxxLxPxxPDYxxL;
(SEQ ID NO: 38)
PxxPxₙYxxL;
(SEQ ID NO: 39)
PxxPxₙDYXXL;
(SEQ ID NO: 40)
PxxPxₙYXₙYxxL;
(SEQ ID NO: 41)
PxxPxₙYxₙDYxxL;
(SEQ ID NO: 42)
PxxPEXₙYxxLxₙPxxPDYxxL;
(SEQ ID NO: 43)
PxxPxxxxPxxPxxxYxxLxPxxPExYxxLxPxxPDYxxL;
(SEQ ID NO: 44)
PxxPxₙPxxPxₙYxxLxₙPxxPExₙ YxxLxₙPxxPDYxxL;
(SEQ ID NO: 45)
PxxPxxxxPxxPxxxYxxLxPxxPExYxxLxPxxPDYxxLxxxx;
and
(SEQ ID NO: 46)
PxxPxₙPxxPXₙYxxLxₙPxxPEXₙYxxLxₙPxxPDYxxLxxxx;

in which “x” and “Xₙ”, respectively, represent any amino acid residue and any one or several amino acid residue(s).

In particular, “n” may be greater than or equal to 1 and less than 50. “n” may, in particular, have any value between 1 and 20.

In some embodiments, the pilot peptide comprises from 6 to 100 amino acid residues, in particular from 20 to 80, from 30 to 70, or from 40 to 60 amino acid residues, for example 40, 41, 42, 43, 44, 45, 46, 47, 48, 49 or 50 amino acid residues.

In some embodiments, the pilot peptide comprises or consists of the amino acid sequence APHFPEISFPPKPDSDYQALLPSAPEIYSHLSPTKPDYINLRPAP (SEQ ID NO: 47) or a variant thereof.

A variant of the amino acid sequence with SEQ ID NO: 47 may comprise an amino acid sequence sharing at least 70% of global sequence identity, preferably at least 75%, 80%, 85%, 90%, 95%, 96%, 97%, 98%, 99% or more of global sequence identity with the amino acid sequence of SEQ ID NO: 47.

Additionally or alternatively, a variant of the amino acid sequence with SEQ ID NO: 47 may comprise an amino acid sequence sharing at least 70% of local sequence identity, preferably at least 75%, 80%, 85%, 90%, 95%, 96%, 97%, 98%, 99% or more of local sequence identity with the amino acid sequence of SEQ ID NO: 47.

Preferably, a variant of the amino acid sequence with SEQ ID NO: 47 retains at least one, two or three YxxL or DYxxL motif(s), and one, two, three or four PxxP motifs.

Even more preferably, a variant of the amino acid sequence of SEQ ID NO: 47 retains three YxxL and/or DYxxL motifs, and four PxxP motifs.

In some embodiments, the chimeric polypeptide further comprises at least one linker.

In some embodiments, the at least one linker connects the amino acid sequence of the protein of interest and the amino acid sequence of the transmembrane domain.

In some embodiments, the at least one linker connects the amino acid sequence of the transmembrane domain to the amino acid sequence of the sub-membrane targeting domain. In some embodiments, the at least one linker connects the amino acid sequence of the pilot peptide to the amino acid sequence of the sub-membrane targeting domain.

In some embodiments, the at least one linker is not cleavable. In some embodiments, the at least one linker is cleavable.

In some embodiments, the at least one linker is a Gly-Ser linker. Examples of Gly/Ser linkers include, but are not limited to, GS linkers, G₂S linkers, G₃S linkers, G₄S linkers, including repeats and combination thereof.

In some embodiments, the at least one linker comprises or consists of a (GGGS)ₙ sequence (SEQ ID NO: 48), wherein n is a positive integer ranging from 1 to 10, preferably from 1 to 5.

In some embodiments, the at least one linker comprises or consists of a (GGGSGGGGS) n sequence (SEQ ID NO: 51), wherein n is a positive integer ranging from 1 to 10, preferably from 1 to 5.

In some embodiments, the at least one linker connecting the amino acid sequence of the protein of interest and the amino acid sequence of the transmembrane domain comprises the sequence SGGGSGGGGSGGGSGGGGSGGGSGGGGSGGGGS (SEQ ID NO: 52). Said linker may be used in a chimeric polypeptide as described hereinabove, wherein the components i), ii), and, iii) when present, are organized in the chimeric polypeptide from N-terminal to C-terminal (such as construct 1 of FIGS. 2, 3 and 4).

In some embodiments, the at least one linker connecting the amino acid sequence of the protein of interest and the amino acid sequence of the transmembrane domain comprises the sequence GGGSGGGGSGGGSGGGGSGGGSGGGGSGGGSG (SEQ ID NO: 53). Said linker may be used in a chimeric polypeptide as described hereinabove, wherein the components i), ii), and, iii) when present, are organized in the chimeric polypeptide from C-terminal to N-terminal (such as constructs 3, 4, 5 of FIGS. 2, 3 and 4).

In some embodiments, when the chimeric polypeptide comprising more than one linker, two or more linkers may be identical or different.

In some embodiments, the chimeric polypeptide comprises an amino acid sequence of a sub-membrane targeting domain.

In some embodiments, the chimeric polypeptide comprises an amino acid sequence of a pilot peptide interacting with the Endosomal Sorting Complexes Required for Transport (ESCRT) cellular machinery and an amino acid sequence of a sub-membrane targeting domain.

In some embodiments, a sub-membrane targeting domain is added in a chimeric polypeptide as described hereinabove, wherein the components i), ii) and, iii) when present, are organized in the chimeric polypeptide from C-terminal to N-terminal (such as constructs 4, 5 of FIGS. 2, 3 and 4).

In some embodiments, the sub-membrane targeting domain is sufficient to allow the chimeric polypeptide to be anchored to the lipid bilayer of cellular or vesicular membranes, preferably via one or more anchoring molecules and/or through interactions such as electrostatic interactions.

Hence, due to its presence in the chimeric polypeptide, the sub-membrane targeting domain allows the chimeric polypeptide, when expressed in a cell, to be anchored to (or anchored in) a cell or vesicular membrane, without the chimeric polypeptide being inserted into said membrane.

In some embodiments, the sub-membrane targeting domain confers to the chimeric polypeptide the property of binding to the inner surface of the cell membrane (i.e., the cytoplasmic side of the cell membrane) and/or to the inner surface of vesicular membranes (i.e., the lumen side of the vesicular membrane).

In some embodiments, the chimeric polypeptide further comprises a sub-membrane targeting domain, preferably wherein the sub-membrane targeting domain is linked to an anchoring molecule.

By “anchoring molecule”, it is meant any molecule capable of being inserted into at least one layer of the lipid bilayer of a cell or vesicular membrane. In particular, the anchoring molecule is a lipid or lipid-containing molecule. The sub-membrane targeting domain is then said to be “lipid-anchored”.

In some embodiments, the anchoring molecule comprises or consists of one or more lipids or lipid-containing molecules, said lipids comprising a hydrophobic carbon chain which allows them to encapsulate in the lipid bilayer of a cell or vesicular membrane.

In some embodiments, the lipids are fatty acids, including, without limitation, myristic acid, palmitic acid, and isoprenoid (such as, e.g., geranyl-geranyl and farnesyl).

In some embodiments, the anchoring molecule is linked to the sub-membrane targeting domain by a covalent bond.

In some embodiments, the anchoring molecule is linked to the sub-membrane targeting domain through a glycine (e.g., in the case of a myristic acid), cysteine or serine amino acid residue of the sub-membrane targeting domain. This link may be through an amide or thioester bond.

In some embodiments, the sub-membrane targeting domain is that of an extrinsic membrane protein or is a variant of the sub-membrane targeting domain of an extrinsic membrane protein.

In some embodiments, the sub-membrane targeting domain comprises or consists of a consensus sequence allowing the attachment (e.g., by acylation or by prenylation) of a fatty acid, and in particular of myristic acid, palmitic acid, or isoprenoid (such as, e.g., geranyl-geranyl and farnesyl).

In some embodiments, the sub-membrane targeting domain comprises or consists of a consensus sequence as follows (M)G-X₁-X₂-X₃-X₄,
wherein X₁, X₂, and X₃ independently from each other denote any amino acid residue,
wherein X₄ denotes an amino acid selected from a serine(S) residue or a cysteine (C) residue and
wherein (M) denotes an initiator methionine which, when located at the N-terminal extremity of the chimeric polypeptide, can be removed in vivo by post-translation processing.

In some embodiments,
X₁ is selected from C, S and L; and/or
X₂ is selected from S, I, V, M and L; and/or
X₃ is selected from K, Q, H, F, C and S; and/or
X₄ is selected from S or C.

In some embodiments, when the chimeric polypeptide described herein comprises a sub-membrane targeting domain linked to an anchoring molecule, it is preferably located at the N-terminal position of the chimeric polypeptide.

In some embodiments, the sub-membrane targeting domain may further comprise several basic amino acid residues, in particular several amino acid residues selected from K, R and H. By “several”, it is meant at least 2, and preferably at least 3 or more. These basic amino acid residues may in particular be involved in interactions with lipids of cell or vesicular membranes, especially with choline and derivatives thereof (e.g., with phosphatidylcholine), and thus make it possible to increase the affinity of the sub-membrane targeting domain for these membranes.

In some embodiments, the basic amino acid residues may be located in the consensus sequence (M)G-X₁-X₂-X₃-X₄ and/or outside this consensus sequence.

Thus, in some embodiments, the sub-membrane targeting domain:
comprises or consists of an amino acid sequence chosen among:

(SEQ ID NO: 55)
(M)GXXKS,
CKXK,
and
(SEQ ID NO: 56)
CKXKXXXXRRR,

wherein X denotes any amino acid residue, and
wherein (M) denotes an initiator methionine which, when located at the N-terminal extremity of the chimeric polypeptide, can be removed in vivo by post-translation processing; or
is a variant of any of these sequences, said variant retaining the ability of the sub-membrane targeting domain to be anchored in the lipid bilayer of a cell or vesicular membrane.

In some embodiments, the sub-membrane targeting domain is derived from a protein of the Src family of proteins. Examples of such proteins include, without limitation, Src, Yes, Lyn, Fyn, Lck, Blk, Fgr, Hck and Yrk proteins (Resh, 1994. Cell. 76 (3): 411-413), and more particularly the N-terminal portion of one of these proteins, such as 15, 16, 17, 18, 19, 20, 21, 22, 23, 24 or 25 N-terminal amino acid residues of one of these proteins.

In some embodiments, the sub-membrane targeting domain is derived from the c-Src or v-Src protein, and preferably from the c-Src.

Alternatively, the sub-membrane targeting domain may be derived from other acylated proteins, such as, e.g., viral capsid proteins, including, without limitation, the human immunodeficiency virus (HIV) MA protein, or filovirus proteins.

In some embodiments, the sub-membrane targeting domain is derived from a Src protein.

In some embodiments, the sub-membrane targeting domain is derived from a Src protein and comprises or consists of one of the following amino acid sequences:

(SEQ ID NO: 57)
(M)GSSKSKPKDPSQRRR,
(SEQ ID NO: 58)
(M)GSSKSKPKDPSQRRRKSR
(SEQ ID NO: 59)
(M)GSSKSKPKDPSQRRRKSRGPGG,

or
a variant of any of these sequences, said variant retaining the ability of the sub-membrane targeting domain to be anchored in the lipid bilayer of a cell or vesicular membrane;
   wherein (M) denotes an initiator methionine which, when located at the N-terminal extremity of the chimeric polypeptide, can be removed in vivo by post-translation processing.

In some embodiments, the sub-membrane targeting domain is derived from a Src protein and comprises or consists of an amino acid sequence with SEQ ID NO: 58 or a variant thereof.

In some embodiments, a variant of the amino acid sequence with SEQ ID NO: 57, 58 or 59 comprises an amino acid sequence sharing at least 70% of global sequence identity, preferably at least 75%, 80%, 85%, 90%, 95%, 96%, 97%, 98%, 99% or more of global sequence identity with the amino acid sequence of SEQ ID NO: 57, 58 or 59.

Additionally or alternatively, a variant of the amino acid sequence with SEQ ID NO: 57, 58 or 59 comprises an amino acid sequence sharing at least 70% of local sequence identity, preferably at least 75%, 80%, 85%, 90%, 95%, 96%, 97%, 98%, 99% or more of local sequence identity with the amino acid sequence of SEQ ID NO: 57, 58 or 59.

In some embodiments, the sub-membrane targeting domain is derived from a Src protein and comprises or consists of an amino acid sequence with SEQ ID NO: 57, 58 or 59 or a variant thereof, preferably with SEQ ID NO: 58; and further comprises one or more anchoring molecules as defined above; in particular, comprises a myristic acid (in the form of a myristyl moiety) linked to the glycine residue at position 2.

In some embodiments, the sub-membrane targeting domain is derived from a Src protein and comprises or consists of an amino acid sequence with SEQ ID NO: 60 or a variant thereof; and further comprises one or more anchoring molecules as defined above; in particular, comprises a myristic acid (in the form of a myristyl moiety) linked to the glycine residue at position 2.

In some embodiments, when the chimeric polypeptide comprises a sub-membrane targeting domain, this sub-membrane targeting domain may be linked to the remaining portions of the chimeric polypeptides through at least one linker.

In some embodiments, the chimeric polypeptide comprises or consists of, from C-terminal to N-terminal:
an amino acid sequence of a protein of interest, wherein said protein of interest is a type II transmembrane protein or a protein from the TNF or TNFR superfamily, as defined hereinabove (such as construct 1 of FIG. 1).

In some embodiments, the chimeric polypeptide comprises or consists of, from C-terminal to N-terminal:
an amino acid sequence of a protein of interest, wherein said protein of interest is a type II transmembrane protein or a protein from the TNF or TNFR superfamily, as defined hereinabove; and
an amino acid sequence of a pilot peptide, as defined hereinabove (such as construct 2 of FIG. 1).

In some embodiments, the chimeric polypeptide comprises or consists of, from C-terminal to N-terminal:
an amino acid sequence of a protein of interest, wherein said protein of interest is a type II transmembrane protein or a protein from the TNF or TNFR superfamily, as defined hereinabove;
optionally, a linker; and
an amino acid sequence of a sub-membrane targeting domain, as defined hereinabove (such as construct 3 of FIG. 1).

In some embodiments, the chimeric polypeptide comprises or consists of, from C-terminal to N-terminal:
an amino acid sequence of a protein of interest, wherein said protein of interest is a type II transmembrane protein or a protein from the TNF or TNFR superfamily, as defined hereinabove;
an amino acid sequence of a pilot peptide, as defined hereinabove;
optionally, a linker; and
an amino acid sequence of a sub-membrane targeting domain, as defined hereinabove (such as construct 4 of FIG. 1).

In some embodiments, the chimeric polypeptide comprises or consists of, from N-terminal to C-terminal:
an amino acid sequence of a protein of interest, wherein said protein of interest is a protein from the TNF or TNFR superfamily, a protein from the TGF-β superfamily, a protein from the neurotrophin family, or a protein from the CTRP family, as defined hereinabove;
optionally, a linker;
an amino acid sequence of a transmembrane domain of a transmembrane protein, as defined hereinabove; and
an amino acid sequence of a pilot peptide, as defined hereinabove (such as construct 1 of FIGS. 2, 3 and 4).

In some embodiments, the chimeric polypeptide comprises or consists of, from C-terminal to N-terminal:
an amino acid sequence of a protein of interest, wherein said protein of interest is a protein from the TNF or TNFR superfamily, a protein from the TGF-β superfamily, a protein from the neurotrophin family, or a protein from the CTRP family, as defined hereinabove;
optionally, a linker; and
an amino acid sequence of a transmembrane domain of a transmembrane protein, as defined hereinabove (such as construct 2 of FIGS. 2, 3 and 4).

In some embodiments, the chimeric polypeptide comprises or consists of, from C-terminal to N-terminal:
an amino acid sequence of a protein of interest, wherein said protein of interest is a protein from the TNF or TNFR superfamily, a protein from the TGF-β superfamily, a protein from the neurotrophin family, or a protein from the CTRP family, as defined hereinabove;
optionally, a linker;
an amino acid sequence of a transmembrane domain of a transmembrane protein, as defined hereinabove;
an amino acid sequence of a pilot peptide, as defined hereinabove (such as construct 3 of FIGS. 2, 3 and 4).

In some embodiments, the chimeric polypeptide comprises or consists of, from C-terminal to N-terminal:
an amino acid sequence of a protein of interest, wherein said protein of interest is a protein from the TNF or TNFR superfamily, a protein from the TGF-β superfamily, a protein from the neurotrophin family, or a protein from the CTRP family, as defined hereinabove;
optionally, a linker;
an amino acid sequence of a transmembrane domain of a transmembrane protein, as defined hereinabove;
optionally, a linker; and
an amino acid sequence of a sub-membrane targeting domain, as defined hereinabove (such as construct 4 of FIGS. 2, 3 and 4).

In some embodiments, the chimeric polypeptide comprises or consists of, from C-terminal to N-terminal:
an amino acid sequence of a protein of interest, wherein said protein of interest is a protein from the TNF or TNFR superfamily, a protein from the TGF-β superfamily, a protein from the neurotrophin family, or a protein from the CTRP family, as defined hereinabove;
optionally, a linker;
an amino acid sequence of a transmembrane domain of a transmembrane protein, as defined hereinabove;
an amino acid sequence of a pilot peptide, as defined hereinabove;
optionally, a linker; and
an amino acid sequence of a sub-membrane targeting domain, as defined hereinabove (such as construct 5 of FIGS. 2, 3 and 4).

Optionally, one or several linker(s) may be added between the components of the chimeric polypeptide of the present invention.

In some embodiments, the chimeric polypeptide comprises or consists of, an amino acid sequence of Neuraminidase N2 as set forth in SEQ ID NO: 1, linked at its N-terminal end to an amino acid sequence as set forth in SEQ ID NO: 61 (such as construct 2 of FIG. 1). Thus, in some embodiments, the chimeric polypeptide comprises or consists of an amino acid sequence as set forth in SEQ ID NO: 68.

In some embodiments, the chimeric polypeptide comprises or consists of, an amino acid sequence of TRAIL as set forth in SEQ ID NO: 2, linked at its N-terminal end to an amino acid sequence as set forth in SEQ ID NO: 61 (such as construct 2 of FIG. 1). Thus, in some embodiments, the chimeric polypeptide comprises or consists of an amino acid sequence as set forth in SEQ ID NO: 69.

In some embodiments, the chimeric polypeptide comprises or consists of, an amino acid sequence of TRAIL external domain as set forth in SEQ ID NO: 3, linked at its N-terminal end to an amino acid sequence as set forth in SEQ ID NO: 65 (such as construct 3 of FIG. 2). Thus, in some embodiments, the chimeric polypeptide comprises or consists of an amino acid sequence as set forth in SEQ ID NO: 76.

In some embodiments, the chimeric polypeptide comprises or consists of, an amino acid sequence of TRAIL TNF-like domain as set forth in SEQ ID NO: 4, linked at its N-terminal end to an amino acid sequence as set forth in SEQ ID NO: 65 (such as construct 3 of FIG. 2). Thus, in some embodiments, the chimeric polypeptide comprises or consists of an amino acid sequence as set forth in SEQ ID NO: 77.

In some embodiments, the chimeric polypeptide comprises or consists of, an amino acid sequence of TNFα as set forth in SEQ ID NO: 5, linked at its N-terminal end to an amino acid sequence as set forth in SEQ ID NO: 61 (such as construct 2 of FIG. 1). Thus, in some embodiments, the chimeric polypeptide comprises or consists of an amino acid sequence as set forth in SEQ ID NO: 70.

In some embodiments, the chimeric polypeptide comprises or consists of, an amino acid sequence of TNFα external domain as set forth in SEQ ID NO: 6, linked at its N-terminal end to an amino acid sequence as set forth in SEQ ID NO: 65 (such as construct 3 of FIG. 2). Thus, in some embodiments, the chimeric polypeptide comprises or consists of an amino acid sequence as set forth in SEQ ID NO: 78.

In some embodiments, the chimeric polypeptide comprises or consists of, an amino acid sequence of TNFα mature domain as set forth in SEQ ID NO: 7, linked at its N-terminal end to an amino acid sequence as set forth in SEQ ID NO: 65 (such as construct 3 of FIG. 2). Thus, in some embodiments, the chimeric polypeptide comprises or consists of an amino acid sequence as set forth in SEQ ID NO: 79.

In some embodiments, the chimeric polypeptide comprises or consists of, an amino acid sequence of CD30L as set forth in SEQ ID NO: 8, linked at its N-terminal end to an amino acid sequence as set forth in SEQ ID NO: 61 (such as construct 2 of FIG. 1). Thus, in some embodiments, the chimeric polypeptide comprises or consists of an amino acid sequence as set forth in SEQ ID NO: 71.

In some embodiments, the chimeric polypeptide comprises or consists of, an amino acid sequence of CD30L external domain as set forth in SEQ ID NO: 9, linked at its N-terminal end to an amino acid sequence as set forth in SEQ ID NO: 65 (such as construct 3 of FIG. 2). Thus, in some embodiments, the chimeric polypeptide comprises or consists of an amino acid sequence as set forth in SEQ ID NO: 80.

In some embodiments, the chimeric polypeptide comprises or consists of, an amino acid sequence of CD137L as set forth in SEQ ID NO: 10, linked at its N-terminal end to an amino acid sequence as set forth in SEQ ID NO: 61 (such as construct 2 of FIG. 1). Thus, in some embodiments, the chimeric polypeptide comprises or consists of an amino acid sequence as set forth in SEQ ID NO: 72.

In some embodiments, the chimeric polypeptide comprises or consists of, an amino acid sequence of CD137L as set forth in SEQ ID NO: 10, linked at its N-terminal end to an amino acid sequence as set forth in SEQ ID NO: 62 (such as construct 3 of FIG. 1). Thus, in some embodiments, the chimeric polypeptide comprises or consists of an amino acid sequence as set forth in SEQ ID NO: 73.

In some embodiments, the chimeric polypeptide comprises or consists of, an amino acid sequence of CD137L as set forth in SEQ ID NO: 10, linked at its N-terminal end to an amino acid sequence as set forth in SEQ ID NO: 63 (such as construct 4 of FIG. 1). Thus, in some embodiments, the chimeric polypeptide comprises or consists of an amino acid sequence as set forth in SEQ ID NO: 74.

In some embodiments, the chimeric polypeptide comprises or consists of, an amino acid sequence of OX40L as set forth in SEQ ID NO: 90, linked at its N-terminal end to an amino acid sequence as set forth in SEQ ID NO: 61 (such as construct 2 of FIG. 1). Thus, in some embodiments, the chimeric polypeptide comprises or consists of an amino acid sequence as set forth in SEQ ID NO: 91.

In some embodiments, the chimeric polypeptide comprises or consists of, an amino acid sequence of TGF-β1 as set forth in SEQ ID NO: 11, linked at its N-terminal end to an amino acid sequence as set forth in SEQ ID NO: 65 (such as construct 3 of FIG. 3). Thus, in some embodiments, the chimeric polypeptide comprises or consists of an amino acid sequence as set forth in SEQ ID NO: 81.

In some embodiments, the chimeric polypeptide comprises or consists of, an amino acid sequence of BMP7 as set forth in SEQ ID NO: 12, linked at its N-terminal end to an amino acid sequence as set forth in SEQ ID NO: 65 (such as construct 3 of FIG. 3). Thus, in some embodiments, the chimeric polypeptide comprises or consists of an amino acid sequence as set forth in SEQ ID NO: 82.

In some embodiments, the chimeric polypeptide comprises or consists of, an amino acid sequence of BMP9 as set forth in SEQ ID NO: 13, linked at its N-terminal end to an amino acid sequence as set forth in SEQ ID NO: 65 (such as construct 3 of FIG. 3). Thus, in some embodiments, the chimeric polypeptide comprises or consists of an amino acid sequence as set forth in SEQ ID NO: 83.

In some embodiments, the chimeric polypeptide comprises or consists of, an amino acid sequence of GDNF as set forth in SEQ ID NO: 14, linked at its N-terminal end to an amino acid sequence as set forth in SEQ ID NO: 65 (such as construct 3 of FIG. 3). Thus, in some embodiments, the chimeric polypeptide comprises or consists of an amino acid sequence as set forth in SEQ ID NO: 84.

In some embodiments, the chimeric polypeptide comprises or consists of, an amino acid sequence of BDNF as set forth in SEQ ID NO: 15, linked at its N-terminal end to an amino acid sequence as set forth in SEQ ID NO: 65 (such as construct 3 of FIG. 3). Thus, in some embodiments, the chimeric polypeptide comprises or consists of an amino acid sequence as set forth in SEQ ID NO: 85.

In some embodiments, the chimeric polypeptide comprises or consists of, an amino acid sequence of CTRP3 as set forth in SEQ ID NO: 16, linked at its C-terminal end to an amino acid sequence as set forth in SEQ ID NO: 64 (such as construct 1 of FIG. 4). Thus, in some embodiments, the chimeric polypeptide comprises or consists of an amino acid sequence as set forth in SEQ ID NO: 75.

In some embodiments, the chimeric polypeptide comprises or consists of, an amino acid sequence of CTRP3 as set forth in SEQ ID NO: 16, linked at its N-terminal end to an amino acid sequence as set forth in SEQ ID NO: 65 (such as construct 3 of FIG. 4). Thus, in some embodiments, the chimeric polypeptide comprises or consists of an amino acid sequence as set forth in SEQ ID NO: 86.

In some embodiments, the chimeric polypeptide comprises or consists of, an amino acid sequence of CTRP3 as set forth in SEQ ID NO: 16, linked at its N-terminal end to an amino acid sequence as set forth in SEQ ID NO: 66 (such as construct 4 of FIG. 4). Thus, in some embodiments, the chimeric polypeptide comprises or consists of an amino acid sequence as set forth in SEQ ID NO: 87.

In some embodiments, the chimeric polypeptide comprises or consists of, an amino acid sequence of CTRP3 as set forth in SEQ ID NO: 16, linked at its N-terminal end to an amino acid sequence as set forth in SEQ ID NO: 67 (such as construct 5 of FIG. 4). Thus, in some embodiments, the chimeric polypeptide comprises or consists of an amino acid sequence as set forth in SEQ ID NO: 88.

The present invention also relates to a nucleic acid encoding the chimeric polypeptide of the present invention, as defined hereinabove.

In some embodiments, the nucleic acid encoding the chimeric polypeptide of the present invention is associated with a lipid nanoparticle or a viral vector. When associated with a lipid nanoparticle, the nucleic acid can be transfected to a subject and when associated to a viral vector, the nucleic acid can be transduced to a subject. In some embodiments, when the nucleic acid is associated to a lipid nanoparticle and transfected to a subject, or associated to a viral vector and transduced to a subject, the nucleic acid allows the production of the chimeric polypeptide in situ. In some embodiments, when the chimeric polypeptide is produced from the nucleic acid in situ, it is directly associated to the subject's extracellular vesicles.

Another object of the present invention is an expression vector comprising the nucleic acid encoding the chimeric polypeptide of the present invention.

In some embodiment, the expression vector comprises a sequence encoding the chimeric polypeptide of the present invention, preferably operably linked to regulatory elements.

Examples of regulatory elements include, but are not limited to, promoters, Kozak consensus initiation sequence, polyadenylation signal, termination sequence (i.e., stop codon), and the like. In particular, the regulatory elements are suitable for expression of the nucleic acid in a cell, such as a bacterium, a yeast, an insect cell, a mammalian cell, or a human cell.

In some embodiments, the expression vector according to the present invention is monocistronic.

By “monocistronic”, it is meant that a single nucleic acid encoding a single protein is expressed in a single expression vector.

In some embodiments, the expression vector according to the present invention is polycistronic.

By “polycistronic”, it is meant that at least two or more nucleic acids, each encoding a single protein, are expressed in a single expression vector.

Another object of the present invention is a cell comprising the nucleic acid encoding the chimeric polypeptide of the invention, or the expression vector comprising the nucleic acid encoding the chimeric polypeptide of the invention.

The present invention further relates to an extracellular vesicle (EV) comprising a chimeric polypeptide, as defined hereinabove.

The present invention also relates to an extracellular vesicle (EV) comprising a nucleic acid encoding the chimeric polypeptide of the present invention, as defined hereinabove.

The present invention also relates to an extracellular vesicle (EV) comprising a chimeric polypeptide, as defined hereinabove, and/or a nucleic acid encoding the chimeric polypeptide of the present invention, as defined hereinabove.

In some embodiments, the extracellular vesicle harbors at its outer surface the protein of interest comprised in the chimeric polypeptide. In some embodiments, the transmembrane domain of the chimeric polypeptide is anchored in the extracellular vesicle lipid bilayer.

In some embodiments, the extracellular vesicle harbors at its outer surface the nucleic acid encoding the chimeric polypeptide, as defined hereinabove.

As used herein, the expression “harbors at its outer surface” means that the protein of interest comprised in the chimeric polypeptide is exposed, partially or completely, outside the extracellular vesicle. This configuration enables the oligomerization of the protein of interest comprised in the chimeric polypeptide, either with other protein of interest of neighboring chimeric polypeptides in the same extracellular vesicle, or with soluble protein of interest.

In some embodiments, the extracellular vesicle is a small extracellular vesicle.

In some embodiments, the extracellular vesicle is an exosome.

In some embodiments, exosomes have a diameter ranging from about 30 nm to about 150 nm, preferably from about 30 nm to about 120 nm, more preferably from about 40 nm to about 80 nm. In some embodiments, exosomes have a diameter ranging from about 30 nm to about 120 nm. In some embodiments, exosomes have a diameter ranging from about 30 nm to about 150 nm.

A further object of the present invention is a population of extracellular vesicles, as defined hereinabove.

In some embodiments, the population of extracellular vesicles is monodisperse in aqueous solutions, preferably in a NaCl 0.9% aqueous solution and/or in PBS.

By “monodisperse”, it is meant that the extracellular vesicles in the population of extracellular vesicles are substantially uniform in size. By “substantially uniform”, it is meant that the extracellular vesicles have a narrow distribution of sizes around an average size. In some embodiments, the extracellular vesicles in NaCl 0.9% aqueous solution and/or in PBS have sizes exhibiting a standard deviation of less than 100% with respect to their average size, such as less than 75%, 50%, 40%, 30%, 20%, 10%, or less than 5%.

In some embodiments, the population of extracellular vesicles further comprises soluble protein of interest in its cytosol, i.e., proteins of interest that are in free form; in other words, not comprised in a chimeric polypeptide according to the invention.

In some embodiments, the population of extracellular vesicle further comprises soluble nucleic acid encoding the chimeric polypeptide, as defined hereinabove, i.e., nucleic acid that is not attached to the extracellular vesicle membrane, in its cytosol.

In some embodiments, the population of extracellular vesicles further comprises additional molecule of interest in its cytosol. In some embodiments, the molecule of interest is a nucleic acid molecule or a small therapeutic molecule.

A further object of the present invention is a method of obtaining an extracellular vesicle or a population of extracellular vesicles comprising a chimeric polypeptide or a nucleic acid encoding the chimeric polypeptide, as defined hereinabove.

General means and methods for obtaining extracellular vesicles or a population of extracellular vesicles are well known in the art. See, e.g., Whitford & Guterstam, 2019. Future Med Chem. 11(10): 1225-1236; Taylor & Shah, 2015. Methods. 87:3-10; Desplantes et al., 2017. Sci Rep. 7(1):1032.

In some embodiments, the method for obtaining an extracellular vesicle or a population of extracellular vesicles comprises a step of producing the extracellular vesicle or the population of extracellular vesicles, as defined hereinabove.

In some embodiments, this step of producing the extracellular vesicle or the population of extracellular vesicles comprises transfecting cells with a nucleic acid encoding the chimeric polypeptide, as defined hereinabove.

In some embodiments, the cells are HEK293T cells or cells from a derivative cell line. In some embodiments, the cells are adipocytes. In some embodiments, the cells are immune cells, including, but not limited to, mastocytes, lymphocytes (such as, e.g., T-cells or B-cells), and dendritic cells. In some embodiments, the cells are stem cells, including, but not limited to, embryonic stem cells, adult stem cells (such as, e.g., hematopoietic stem cells, mammary stem cells, intestinal stem cells, mesenchymal stem cells, endothelial stem cells, neural stem cells, olfactory adult stem cells, or neural crest stem cells), cancer stem cells, induced pluripotent stem cells (iPSC) and induced stem cells (iSC).

In some embodiments, the method for obtaining an extracellular vesicle or a population of extracellular vesicles further comprises a step of culturing the transfected cells for a time sufficient to allow extracellular vesicle production, preferably in a medium devoid of extracellular vesicles (i.e., a serum-free medium, a medium supplemented with extracellular vesicle-depleted serum, or a medium supplemented with extracellular vesicle-depleted platelet lysate).

In some embodiments, the method for obtaining an extracellular vesicle or a population of extracellular vesicles further comprises a step of purifying said extracellular vesicle or population of extracellular vesicles.

In some embodiments, the step of purifying said extracellular vesicle or population of extracellular vesicles comprises clarification (such as, e.g., by centrifugation or by depth-filtration), filtration, ultra-filtration, diafiltration, size-exclusion purification and/or ion exchange chromatography of the transfected cell culture supernatant. Other methods to purify extracellular vesicle or population of extracellular vesicles include, without limitation, ultra-centrifugation, tangential flow filtration (TFF) and BE-SEC chromatography.

In some embodiments, the extracellular vesicle or population of extracellular vesicles of the present invention is purified. Thus, the present invention also relates to a purified extracellular vesicle or population of extracellular vesicles.

Methods for purification are well-known by the skilled artisan in the art and includes, without limitation, the methods of purification as described hereinabove.

In some embodiments, the extracellular vesicle or population of extracellular vesicles of the present invention is purified by ultra-centrifugation to obtain semi-purified extracellular vesicle or population of extracellular vesicles. Thus, the present invention also relates to a semi-purified extracellular vesicle or population of extracellular vesicles.

As used herein, a semi-purified extracellular vesicle or population of extracellular vesicles comprises the extracellular vesicle or the population of extracellular vesicles, the proteins anchored in the membrane of the extracellular vesicle or the population of extracellular vesicles or tightly associated with the extracellular vesicle or the population of extracellular vesicles, as well as the crown of proteins associated with the extracellular vesicle or the population of extracellular vesicles (see FIG. 5B).

In some embodiments, the extracellular vesicle or population of extracellular vesicles of the present invention is purified by tangential flow filtration and chromatography, in particular SEC and BE-SEC chromatography, to obtain ultra-purified extracellular vesicle or population of extracellular vesicles. Thus, the present invention also relates to an ultra-purified extracellular vesicle or population of extracellular vesicles.

As used herein, an ultra-purified extracellular vesicle or population of extracellular vesicles comprises the extracellular vesicle or the population of extracellular vesicles, and the proteins anchored in the membrane of the extracellular vesicle or the population of extracellular vesicles or tightly associated with the extracellular vesicle or the population of extracellular vesicles (see FIG. 5C).

The present invention further relates to a composition comprising, consisting essentially of, or consisting of the chimeric polypeptide, the nucleic acid, the expression vector, the cell, the extracellular vesicle or the population of extracellular vesicles, as defined hereinabove.

As used herein, “consisting essentially of”, with reference to a composition, means that the chimeric polypeptide, the nucleic acid, the expression vector, the cell, the extracellular vesicle or the population of extracellular vesicles is the only one therapeutic agent or agent with a biologic activity within said composition.

In some embodiments, the composition further comprises soluble protein of interest, i.e., proteins of interest that are in free form, as already defined above.

In some embodiments, the composition is a pharmaceutical composition and further comprises at least one pharmaceutically acceptable excipient.

Consequently, another object of the present invention is a pharmaceutical composition comprising, consisting essentially of or consisting of the chimeric polypeptide, the nucleic acid, the expression vector, the cell, the extracellular vesicle or the population of extracellular vesicles, as defined hereinabove, and at least one pharmaceutically acceptable excipient.

The term “pharmaceutically acceptable excipient” includes any and all solvents, diluents, dispersion media, coatings, antibacterial and antifungal agents, isotonic and absorption delaying agents and the like. Said excipient does not produce an adverse, allergic or other untoward reaction when administered to an animal, preferably a human. For human administration, preparations should meet sterility, pyrogenicity, and general safety and purity standards as required by regulatory offices, such as, for example, FDA Office or EMA.

Pharmaceutically acceptable excipients that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of vegetable oil saturated fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (e.g., sodium carboxymethylcellulose), polyethylene glycol, polyacrylates, waxes, polyethylene-polyoxypropylene-block polymers, polyethylene glycol and wool fat.

In some embodiments, the pharmaceutical composition further comprises soluble protein of interest, i.e., proteins of interest that are in free form, as already defined above.

In some embodiments, the pharmaceutical composition further comprises soluble nucleic acid encoding the chimeric polypeptide.

The present invention further relates to a medicament comprising, consisting essentially of or consisting of the chimeric polypeptide, the nucleic acid, the expression vector, the cell, the extracellular vesicle or the population of extracellular vesicles, as defined hereinabove.

In some embodiments, the medicament further comprises soluble protein of interest, i.e., proteins of interest that are in free form, as already defined above.

In some embodiments, the medicament further comprises soluble nucleic acid encoding the chimeric polypeptide.

In some embodiments, the composition, the pharmaceutical composition or the medicament comprises a purified extracellular vesicle or population of extracellular vesicles as defined hereinabove.

The present invention further relates to a kit-of-parts comprising, in a first part, the extracellular vesicle or the population of extracellular vesicle, as defined hereinabove; and, in a second part, soluble protein of interest, i.e., proteins of interest that are in free form, or soluble nucleic acid encoding the chimeric polypeptide, as already defined above.

In some embodiments, the two parts of the kit-of-parts are intended for simultaneous use, or for sequential use in any order.

The present invention further relates to the chimeric polypeptide, the nucleic acid, the expression vector, the cell, the extracellular vesicle or the population of extracellular vesicles, the composition, the pharmaceutical composition, the medicament or the kit-of-parts, as defined hereinabove, for use as a drug or medicament.

The present invention further relates to the chimeric polypeptide, the nucleic acid, the expression vector, the cell, the extracellular vesicle or the population of extracellular vesicles, the composition, the pharmaceutical composition, the medicament or the kit-of-parts, as defined hereinabove, for use in the treatment of a disease, disorder or condition.

The present invention further relates to the chimeric polypeptide, the nucleic acid, the expression vector, the cell, the extracellular vesicle or the population of extracellular vesicles, the composition, the pharmaceutical composition, the medicament or the kit-of-parts, as defined hereinabove, for treating or for use in treating a disease, disorder or condition in a subject in need thereof.

The present invention further relates to the use of the chimeric polypeptide, the nucleic acid, the expression vector, the cell, the extracellular vesicle or the population of extracellular vesicles, the composition, the pharmaceutical composition, the medicament or the kit-of-parts, as defined hereinabove, in the manufacture of a medicament for treating a disease, disorder or condition in a subject in need thereof.

The present invention further relates to the use of the chimeric polypeptide, the nucleic acid, the expression vector, the cell, the extracellular vesicle or the population of extracellular vesicles, the composition, the pharmaceutical composition, the medicament or the kit-of-parts, as defined hereinabove, for treating a disease, disorder or condition in a subject in need thereof.

The present invention further relates to a method for treating a disease, disorder or condition in a subject in need thereof, comprising or consisting of administering the chimeric polypeptide, the nucleic acid, the expression vector, the cell, the extracellular vesicle or the population of extracellular vesicles, the population of extracellular vesicles, the composition, the pharmaceutical composition, the medicament or the kit-of-parts, as defined hereinabove, to the subject.

The present invention further relates to a method for treating a disease, disorder or condition in a subject in need thereof, comprising or consisting of administering a therapeutically effective amount of the chimeric polypeptide, the nucleic acid, the expression vector, the cell, the extracellular vesicle or the population of extracellular vesicles, the population of extracellular vesicles, the composition, the pharmaceutical composition, the medicament or the kit-of-parts, as defined hereinabove, to the subject.

In some embodiments, the disease, disorder or condition is selected from neurodegenerative diseases, auto-immune diseases, viral infections, T-cell immune responses, NK-cell immune responses, homeostatic imbalance associated diseases, diabetes, obesity and associated metabolic disease, insulin resistance, cardiovascular diseases, inflammatory conditions, hormonal cancers, and cancer progression.

Examples of neurodegenerative diseases include, but are not limited to, Alzheimer's disease, amyotrophic lateral sclerosis (ALS), Friedreich ataxia, Huntington's disease, Lewy body disease, Parkinson's disease, Spinal muscular atrophy, multiple sclerosis (MS), multiple system atrophy, and prion diseases.

As used herein, the term “autoimmune disease” refers to a disease in which the immune system produces an immune response (e.g., a B cell or a T cell response) against an antigen that is part of the normal host (that is an auto-antigen), with consequent injury to tissues. In an autoimmune disease, the immune system of the host fails to recognize a particular antigen as “self” and an immune reaction is mounted against the host's tissues expressing the antigen.

Exemplary autoimmune diseases contemplated in the present invention include, but are not limited to, rheumatoid arthritis, juvenile oligoarthritis, collagen-induced arthritis, adjuvant-induced arthritis, Sjogren's syndrome, multiple sclerosis, experimental autoimmune encephalomyelitis, inflammatory bowel disease (including Crohn's disease and ulcerative colitis), autoimmune gastric atrophy, pemphigus vulgaris, psoriasis, vitiligo, type 1 diabetes, non-obese diabetes, myasthenia gravis, Grave's disease, Hashimoto's thyroiditis, sclerosing cholangitis, sclerosing sialadenitis, systemic lupus erythematosis, autoimmune thrombocytopenia purpura, Goodpasture's syndrome, Addison's disease, systemic sclerosis, polymyositis, dermatomyositis, acquired hemophilia, thrombotic thrombocytopenia purpura, uveitis, IgG4-associated autoimmune diseases.

As used herein the term “viral infection” refers to any disease or condition caused by a virus.

Examples of viral infection include, but are not limited to, common cold caused by rhinovirus, flu caused by influenza virus, COVID-19 caused by SARS-COV-2 virus, lung infection caused by respiratory syncytial virus, gastroenteritis caused by norovirus, rotavirus or astrovirus, hepatitis caused by hepatitis viruses, hemorrhagic fevers caused by Ebola virus, Yellow fever caused by arbovirus, Dengue fever caused by dengue viruses, acquired immune deficiency syndrome (AIDS) caused by human immunodeficiency virus (HIV), cervical cancer caused by human papillomaviruses (HPV), genital herpes caused by herpes viruses (HSV), chickenpox caused by varicella-zoster virus, measles caused by measles virus, rubella caused by RuV virus, congenital viral infections caused cytomegalovirus (CMV) or zika virus, neurological infections caused by West Nile virus, poliovirus or RABV virus.

As used herein the term “inflammatory conditions” refers to a vast array of disorders and conditions that are characterized by inflammation. Symptoms of inflammatory conditions can include chronic pain, swelling, redness, joint and muscle stiffness, loss of function and movement in the affected area. Inflammatory disorders refer to many heterogeneous conditions that may have the following pathophysiological characteristics:
an inflammatory response to an unidentified agent that involves different tissues and organs, a response depending on genetic variability in the response characteristics of immune cells such as antigen-presenting cells, B and T lymphocytes,
production of autoantibodies (natural and pathogenic autoantibodies) to the exogenous or endogenous antigen,
production of antigen-specific inflammatory cells, such as lymphocytes and T-cells, production and deposition of abnormal protein and other inflammatory products in different tissues eliciting further inflammatory and immune responses,
these responses include inflammation of vessels in the surrounding tissue (i.e., vasculitis) and/or
production of pro-inflammatory and anti-inflammatory mediators.

As used herein, the term “cancer” has its general meaning in the art and in particular refers to a disease caused by an uncontrolled division of abnormal cells. The term “cancer” encompasses solid tumors and blood cancers, and encompasses both primary and metastatic cancers.

Examples of cancers include, but are not limited to, cancer cells from the bladder, blood, bone, bone marrow, brain, breast, colon, esophagus, gastrointestinal, gum, head, kidney, liver, lung, nasopharynx, neck, ovary, prostate, pancreatic, skin, stomach, testis, tongue, or uterus.

As used herein, the term “hormonal cancer” refers to cancers that are dependent or sensitives to hormones. Examples of hormonal cancer include but are not limited to breast cancer, prostate cancer, endometrial carcinoma, ovarian carcinoma, testis carcinoma and thyroid carcinoma.

Examples of cardiovascular diseases include, but are not limited to, myocardial and cerebral infarction, acute myocardial infarction, ischemia, coronary heart disease, acute coronary syndrome, stroke, aneurysm, stable or effort angina pectoris, cardiomyopathy, hypertensive heart disease, heart failure (chronic and acute), cor pulmonale, cardiac dysrhythmias, inflammatory heart disease such as endocarditis, myocarditis, peripheral arterial disease, SIRS-associated myocardial and vascular dysfunction, atherosclerosis.

The present invention also relates to an extracellular vesicle harboring the protein of interest exposed at its outer surface, or a population thereof, for use in the treatment of a disease, disorder or condition selected from neurodegenerative diseases, auto-immune diseases, viral infections, T-cell immune responses, NK-cell immune response, homeostasis, diabetes, obesity and associated metabolic disease, insulin resistance, cardiovascular diseases, inflammatory conditions, hormonal cancers and cancer progression.

In some embodiments, the extracellular vesicle is partially or totally coated with recombinant protein of interest.

By “recombinant protein of interest”, it is meant exogenous protein of interest which is not endogenously produced by a cell. Extracellular vesicles partially or totally coated with recombinant protein of interest can be obtained by contacting extracellular vesicles with the protein of interest, either in cellulo (e.g., by transfecting an extracellular vesicle-producing cell with a nucleic acid encoding the protein of interest, thereby having the cell produce exogenous protein of interest) or ex cellulo (e.g., by providing the protein of interest in protein form, previously produced in a suitable recombinant expression system and further purified).

The present invention also relates to an extracellular vesicle harboring the nucleic acid encoding the chimeric polypeptide of the present invention, as defined hereinabove, exposed at its outer surface, or a population thereof, for use in the treatment of a disease, disorder or condition selected from neurodegenerative diseases, auto-immune diseases, viral infections, T-cell immune responses, NK-cell immune response, homeostasis, diabetes, obesity and associated metabolic disease, insulin resistance, cardiovascular diseases, inflammatory conditions, hormonal cancers and cancer progression.

In some embodiments, the extracellular vesicle is partially or totally coated with the nucleic acid.

By “coated”, it is implied that the protein of interest or the nucleic acid encoding the chimeric polypeptide is exposed at the outer surface of the extracellular vesicle, to which it is bound through any suitable type of interaction with external components of the vesicle (such as, without limitation, electrostatic interactions, protein-protein interactions, protein-lipid interactions, etc.).

In some embodiments, the chimeric polypeptide, the nucleic acid, the expression vector, the cell, the extracellular vesicle, the population of extracellular vesicles, the composition, the pharmaceutical composition, the medicament or the components of the kit-of-parts, as defined hereinabove, is/are formulated for administration to a subject in need thereof.

In some embodiments, administration to a subject can be performed parenterally, by inhalation spray, rectally, nasally, or via an implanted reservoir. The term “administration” includes, inter alia, subcutaneous, intravenous, intramuscular, intra-articular, intra-synovial, intrasternal, intrathecal, intrahepatic, intralesional and intracranial injection or infusion techniques.

In some embodiments, the chimeric polypeptide, the nucleic acid, the expression vector, the cell, the extracellular vesicle, the population of extracellular vesicles, the composition, the pharmaceutical composition, the medicament or the components of the kit-of-parts, as defined hereinabove, is/are to be administered to a subject in need thereof in a therapeutically effective amount.

It will be however understood that the total daily usage of the chimeric polypeptide, the nucleic acid, the expression vector, the cell, the extracellular vesicle, the population of extracellular vesicles, the composition, the pharmaceutical composition, the medicament or the components of the kit-of-parts, as defined hereinabove, will be decided by the attending physician within the scope of sound medical judgment.

In particular, the specific therapeutically effective dose level for any particular patient will depend upon a variety of factors including the disease being treated and the severity of the disease; activity of the chimeric polypeptide, the nucleic acid, the expression vector, the cell, the extracellular vesicle, the population of extracellular vesicles, the composition, the pharmaceutical composition, the medicament or the components of the kit-of-parts, as defined hereinabove, employed; the age, body weight, general health, sex and diet of the subject; the time of administration, route of administration, and rate of excretion of the chimeric polypeptide, the nucleic acid, the expression vector, the cell, the extracellular vesicle, the population of extracellular vesicles, the composition, the pharmaceutical composition, the medicament or the components of the kit-of-parts, as defined hereinabove, employed; the duration of the treatment; drugs used in combination or coincidental with the chimeric polypeptide, the nucleic acid, the expression vector, the cell, the extracellular vesicle, the population of extracellular vesicles, the composition, the pharmaceutical composition, the medicament or the components of the kit-of-parts, as defined hereinabove, employed; and like factors well-known in the medical arts. The total dose required for each treatment may be administered by multiple doses or in a single dose.

The present invention also relates to the in vitro or in vivo use of the chimeric polypeptide, the nucleic acid, the expression vector, the cell, the extracellular vesicle or the population of extracellular vesicles of the present invention in non-therapeutic methods.

In some embodiments, the chimeric polypeptide, the nucleic acid, the expression vector, the cell, the extracellular vesicle or the population of extracellular vesicles of the present invention is used in vitro or in vivo for assessing the function or biological activity of the protein of interest in various biological processes.

In some embodiments, the chimeric polypeptide, the nucleic acid, the expression vector, the cell, the extracellular vesicle or the population of extracellular vesicles of the present invention is used with in vitro or in vivo assays to determine the effect of the protein of interest in a given biological process.

In some embodiments, the chimeric polypeptide, the nucleic acid, the expression vector, the cell, the extracellular vesicle or the population of extracellular vesicles of the present invention is used with in vitro or in vivo assays to develop therapeutic molecules.

In some embodiments, the therapeutic molecule is a small molecule, an antibody, an agonist, or an antagonist.

### BRIEF DESCRIPTION OF THE DRAWINGS

FIG. 1 is a schematic representation showing 4 exemplary constructs of the chimeric polypeptide comprising a type II transmembrane protein as protein of interest according to the present invention.

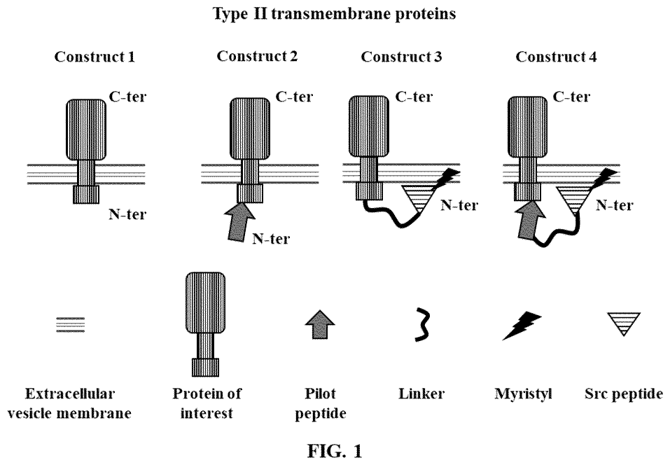

FIG. 2 is a schematic representation showing 5 exemplary constructs of the chimeric polypeptide comprising a protein from the TNF or TNF receptor superfamily as a protein of interest according to the present invention.

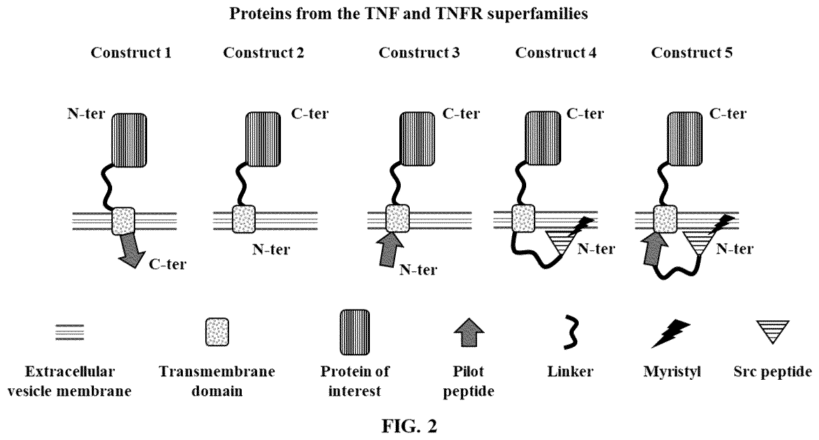

FIG. 3 is a schematic representation showing 5 exemplary constructs of the chimeric polypeptide comprising a protein from the TGF-β superfamily as a protein of interest according to the present invention.

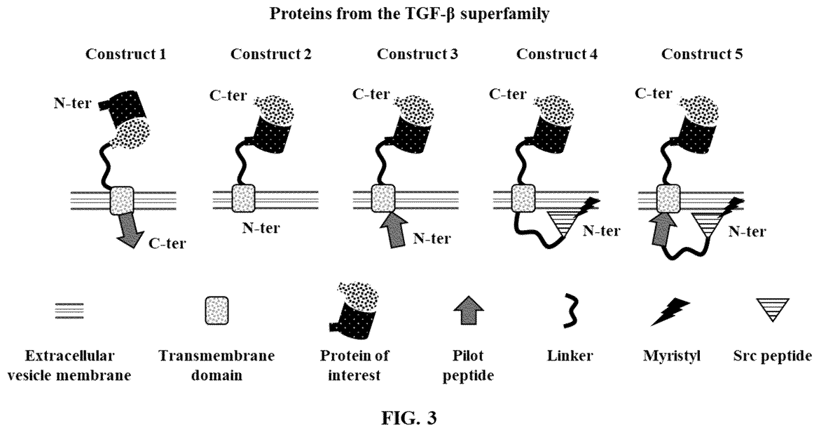

FIG. 4 is a schematic representation showing 5 exemplary constructs of the chimeric polypeptide comprising a protein from the CTRP family as a protein of interest according to the present invention.

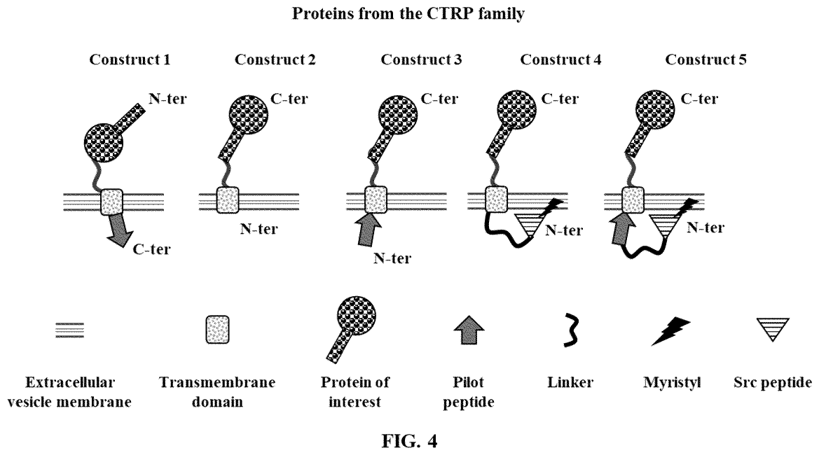

FIG. 5 is a schematic representation of EVs and of the environment of EVs in culture medium (FIG. 5A), of semi-purified EVs (FIG. 5B) and of ultra-purified EVs (FIG. 5C). FIG. 5A: EVs in culture medium are associated with proteins and with contaminants. FIG. 5B: EVs semi-purified by ultracentrifugation are associated with a crown of associated proteins. FIG. 5C: EVs ultra-purified by TFF (tangential flow filtration) and chromatography are associated with proteins anchored in the membrane or tightly associated with the membrane.

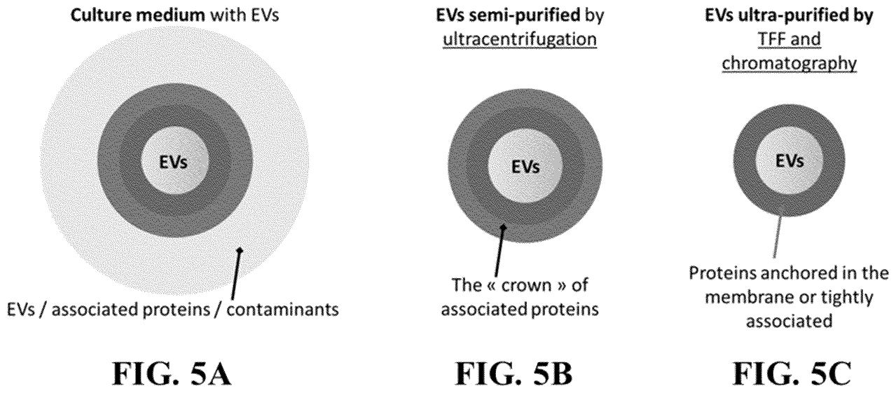

FIGS. 6A-E is a combination of photographs of immunotransfert analysis and a graph showing the expression of CD137L constructs in extracellular vesicles. HEK293T cells were transfected with DNA encoding different CD137L constructs: (i) DNA encoding wild-type CD137L protein (encoding the polypeptide with SEQ ID NO: 10), (ii) DNA encoding CD137L construct 1 comprising a pilot peptide fused upstream of CD137L (encoding the polypeptide with SEQ ID NO: 72), (iii) DNA encoding CD137L construct 2 comprising a Src peptide fused upstream of CD137L (encoding the polypeptide with SEQ ID NO: 73), or (iv) DNA encoding 137L construct 3 comprising both Src peptide fused and pilot peptide fused upstream of CD137L (encoding the polypeptide with SEQ ID NO: 74) in order to express and sort the constructs with extracellular vesicles. Extracellular vesicles produced by cells transfected by each of the DNA constructs and also by a control DNA were purified, lysed and their protein contents were separated by polyacrylamide gel electrophoresis and analyzed by immunotransfert (WB). FIG. 6A represents the proteins of each extracellular vesicle lysate analyzed using Mini-Protean TGX Stain Free Gels, 4-15% (BioRad). FIG. 6B represents WB probed with anti-Alix antibodies. FIGS. 6C-D represent the same western blot probed with anti-CD137L antibodies with an exposure time of 85 sec (FIG. 6C) or an exposure time of 139 sec (FIG. 6D). FIG. 6E is a histogram representing the amount of each CD137L construct relative to Alix amount (ratio of amount of pixels in CD137L construct bands/amount of pixels in Alix band) in each lane of the presented western blots.

FIGS. 7A-B is a combination of graphs showing the median size and expression of CD81 in extracellular vesicles harboring CD137L protein. FIG. 7A shows the concentration and size distribution of extracellular vesicles harboring CD137L protein. FIG. 7B shows the detection by ELISA of CD81 on extracellular vesicles produced by untransfected cells (EVs standards), and extracellular vesicles harboring CD137L.

FIG. 8 is a photograph of immunotransfert analysis showing the expression of OX40L construct in cells and extracellular vesicles. HEK293T cells were stably transfected with DNA encoding an OX40L construct comprising a pilot peptide fused upstream of OX40L (SEQ ID NO: 91) to express the fusion protein and sort it with extracellular vesicles. Cell pellets were prepared and extracellular vesicles were purified from conditioned culture media. Cells and extracellular vesicles were lysed either under reducing conditions or non-reducing (native) conditions, as indicated, and then submitted or not to deglycosylation using PNGase. Lysates were separated by polyacrylamide gel electrophoresis followed by immunotransfert analysis (WB). WB were probed with anti-pilot peptide rabbit polyclonal antibodies.

TABLE OF
SEQUENCES

| SEQ | | |
|---|---|---|
| ID | | |
| NO | Sequence function | Sequence |
| 1 | Influenza A virus H3N2 | MNPNQKIITIGSVSLTISTICFFMQIAILITTVTLHFKQ |
| | neuraminidase N2 | YEFNSPPNNQVMLCEPTIIERNITEIVYLTNTTIEKEI |
| | | CPKPAEYRNWSKPQCDITGFAPFSKDNSIRLSAGGD |
| | | IWVTREPYVSCDPDKCYQFALGQGTTLNNVHSNNT |
| | | VRDRTPYRTLLMNELGVPFHLGTKQVCIAWSSSSC |
| | | HDGKAWLHVCITGDDKNATASFIYNGRLVDSVVS |
| | | WSKEILRTQESECVCINGTCTVVMTDGSASGKADT |
| | | KILFIEEGKVVHTSTLSGSAQHVEECSCYPRYPGVR |
| | | CVCRDNWKGSNRPIVDINIKDHSIVSSYVCSGLVGD |
| | | TPRKNDSSSSSHCLDPNNEEGGHGVKGWAFDDGN |
| | | DVWMGRTINETSRLGYETFKVIEGWSNPKSKLQIN |
| | | RQVIVDRGDRSGYSGIFSVEGKSCINRCFYVELIRGR |
| | | KEETEVLWTSNSIVVFCGTSGTYGTGSWPDGADLN |
| | | LMPI |
| 2 | TRAIL | MAMMEVQGGPSLGQTCVLIVIFTVLLQSLCVAVTY |
| | | VYFTNELKQMQDKYSKSGIACFLKEDDSYWDPND |
| | | EESMNSPCWQVKWQLRQLVRKMILRTSEETISTVQ |
| | | EKQQNISPLVRERGPQRVAAHITGTRGRSNTLSSPN |
| | | SKNEKALGRKINSWESSRSGHSFLSNLHLRNGELVI |
| | | HEKGFYYIYSQTYFRFQEEIKENTKNDKQMVQYIY |
| | | KYTSYPDPILLMKSARNSCWSKDAEYGLYSIYQGGI |
| | | FELKENDRIFVSVTNEHLIDMDHEASFFGAFLVG |
| 3 | TRAIL external domain | TNELKQMQDKYSKSGIACFLKEDDSYWDPNDEES |
| | | MNSPCWQVKWQLRQLVRKMILRTSEETISTVQEKQ |
| | | QNISPLVRERGPQRVAAHITGTRGRSNTLSSPNSKN |
| | | EKALGRKINSWESSRSGHSFLSNLHLRNGELVIHEK |
| | | GFYYIYSQTYFRFQEEIKENTKNDKQMVQYIYKYTS |
| | | YPDPILLMKSARNSCWSKDAEYGLYSIYQGGIFELK |
| | | ENDRIFVSVTNEHLIDMDHEASFFGAFLVG |
| 4 | TRAIL TNF-like | SWESSRSGHSFLSNLHLRNGELVIHEKGFYYIYSQT |
| | domain | YFRFQEEIKENTKNDKQMVQYIYKYTSYPDPILLMK |
| | | SARNSCWSKDAEYGLYSIYQGGIFELKENDRIFVSV |
| | | TNEHLIDMDHEASFFGAFLVG |
| 5 | TNFα | MSTESMIRDVELAEEALPKKTGGPQGSRRCLFLSLF |
| | | SFLIVAGATTLFCLLHFGVIGPQREEFPRDLSLISPLA |
| | | QAVRSSSRTPSDKPVAHVVANPQAEGQLQWLNRR |
| | | ANALLANGVELRDNQLVVPSEGLYLIYSQVLFKGQ |
| | | GCPSTHVLLTHTISRIAVSYQTKVNLLSAIKSPCQRE |
| | | TPEGAEAKPWYEPIYLGGVFQLEKGDRLSAEINRPD |
| | | YLDFAESGQVYFGIIAL |
| 6 | TNFα external domain | GPQREEFPRDLSLISPLAQAVRSSSRTPSDKPVAHVV |
| | | ANPQAEGQLQWLNRRANALLANGVELRDNQLVVP |
| | | SEGLYLIYSQVLFKGQGCPSTHVLLTHTISRIAVSYQ |
| | | TKVNLLSAIKSPCQRETPEGAEAKPWYEPIYLGGVF |
| | | QLEKGDRLSAEINRPDYLDFAESGQVYFGIIAL |
| 7 | TNFα mature domain | LQWLNRRANALLANGVELRDNQLVVPSEGLYLIYS |
| | | QVLFKGQGCPSTHVLLTHTISRIAVSYQTKVNLLSAI |
| | | KSPCQRETPEGAEAKPWYEPIYLGGVFQLEKGDRL |
| | | SAEINRPDYLDFAESGQVYFGIIAL |
| 8 | CD30L | MDPGLQQALNGMAPPGDTAMHVPAGSVASHLGTT |
| | | SRSYFYLTTATLALCLVFTVATIMVLVVQRTDSIPN |
| | | SPDNVPLKGGNCSEDLLCILKRAPFKKSWAYLQVA |
| | | KHLNKTKLSWNKDGILHGVRYQDGNLVIQFPGLYF |
| | | IICQLQFLVQCPNNSVDLKLELLINKHIKKQALVTVC |
| | | ESGMQTKHVYQNLSQFLLDYLQVNTTISVNVDTFQ |
| | | YIDTSTFPLENVLSIFLYSNSD |
| 9 | CD30L external domain | QRTDSIPNSPDNVPLKGGNCSEDLLCILKRAPFKKS |
| | | WAYLQVAKHLNKTKLSWNKDGILHGVRYQDGNL |
| | | VIQFPGLYFIICQLQFLVQCPNNSVDLKLELLINKHIK |
| | | KQALVTVCESGMQTKHVYQNLSQFLLDYLQVNTTI |
| | | SVNVDTFQYIDTSTFPLENVLSIFLYSNSD |
| 10 | CD137L | MEYASDASLDPEAPWPPAPRARACRVLPWALVAG |
| | | LLLLLLLAAACAVFLACPWAVSGARASPGSAASPR |
| | | LREGPELSPDDPAGLLDLRQGMFAQLVAQNVLLID |
| | | GPLSWYSDPGLAGVSLTGGLSYKEDTKELVVAKAG |
| | | VYYVFFQLELRRVVAGEGSGSVSLALHLQPLRSAA |
| | | GAAALALTVDLPPASSEARNSAFGFQGRLLHLSAG |
| | | QRLGVHLHTEARARHAWQLTQGATVLGLFRVTPEI |
| | | PAGLPSPRSE |
| 11 | TGF-β1 | LSTCKTIDMELVKRKRIEAIRGQILSKLRLASPPSQG |
| | | EVPPGPLPEAVLALYNSTRDRVAGESAEPEPEPEAD |
| | | YYAKEVTRVLMVETHNEIYDKFKQSTHSIYMFFNT |
| | | SELREAVPEPVLLSRAELRLLRLKLKVEQHVELYQK |
| | | YSNNSWRYLSNRLLAPSDSPEWLSFDVTGVVRQW |
| | | LSRGGEIEGFRLSAHCSCDSRDNTLQVDINGFTTGR |
| | | RGDLATIHGMNRPFLLLMATPLERAQHLQSSRHRR |
| | | ALDTNYCFSSTEKNCCVRQLYIDFRKDLGWKWIHE |
| | | PKGYHANFCLGPCPYIWSLDTQYSKVLALYNQHNP |
| | | GASAAPCCVPQALEPLPIVYYVGRKPKVEQLSNMI |
| | | VRSCKCS |
| 12 | BMP7 | DFSLDNEVHSSFIHRRLRSQERREMQREILSILGLPH |
| | | RPRPHLQGKHNSAPMFMLDLYNAMAVEEGGGPGG |
| | | QGFSYPYKAVFSTQGPPLASLQDSHFLTDADMVMS |
| | | FVNLVEHDKEFFHPRYHHREFRFDLSKIPEGEAVTA |
| | | AEFRIYKDYIRERFDNETFRISVYQVLQEHLGRESDL |
| | | FLLDSRTLWASEEGWLVFDITATSNHWVVNPRHNL |
| | | GLQLSVETLDGQSINPKLAGLIGRHGPQNKQPFMV |
| | | AFFKATEVHFRSIRSTGSKQRSQNRSKTPKNQEALR |
| | | MANVAENSSSDQRQACKKHELYVSFRDLGWQDWI |
| | | IAPEGYAAYYCEGECAFPLNSYMNATNHAIVQTLV |
| | | HFINPETVPKPCCAPTQLNAISVLYFDDSSNVILKKY |
| | | RNMVVRACGCH |
| 13 | BMP9 | KPLQSWGRGSAGGNAHSPLGVPGGGLPEHTFNLK |
| | | MFLENVKVDFLRSLNLSGVPSQDKTRVEPPQYMID |
| | | LYNRYTSDKSTTPASNIVRSFSMEDAISITATEDFPF |
| | | QKHILLFNISIPRHEQITRAELRLYVSCQNHVDPSHD |
| | | LKGSVVIYDVLDGTDAWDSATETKTFLVSQDIQDE |
| | | GWETLEVSSAVKRWVRSDSTKSKNKLEVTVESHR |
| | | KGCDTLDISVPPGSRNLPFFVVFSNDHSSGTKETRLE |
| | | LREMISHEQESVLKKLSKDGSTEAGESSHEEDTDGH |
| | | VAAGSTLARRKRSAGAGSHCQKTSLRVNFEDIGWD |
| | | SWIIAPKEYEAYECKGGCFFPLADDVTPTKHAIVQT |
| | | LVHLKFPTKVGKACCVPTKLSPISVLYKDDMGVPT |
| | | LKYHYEGMSVAECGCR |
| 14 | GDNF | FPLPAGKRPPEAPAEDRSLGRRRAPFALSSDSNMPE |
| | | DYPDQFDDVMDFIQATIKRLKRSPDKQMAVLPRRE |
| | | RNRQAAAANPENSRGKGRRGQRGKNRGCVLTAIH |
| | | LNVTDLGLGYETKEELIFRYCSGSCDAAETTYDKIL |
| | | KNLSRNRRLVSDKVGQACCRPIAFDDDLSFLDDNL |
| | | VYHILRKHSAKRCGCI |
| 15 | BDNF | APMKEANIRGQGGLAYPGVRTHGTLESVNGPKAGS |
| | | RGLTSLADTFEHVIEELLDEDQKVRPNEENNKDAD |
| | | LYTSRVMLSSQVPLEPPLLFLLEEYKNYLDAANMS |
| | | MRVRRHSDPARRGELSVCDSISEWVTAADKKTAV |
| | | DMSGGTVTVLEKVPVSKGQLKQYFYETKCNPMGY |
| | | TKEGCRGIDKRHWNSQCRTTQSYVRALTMDSKKRI |
| | | GWRFIRIDTSCVCTLTIKRGR |
| 16 | CTRP3 | QDEYMESPQTGGLPPDCSKCCHGDYSFRGYQGPPG |
| | | PPGPPGIPGNHGNNGNNGATGHEGAKGEKGDKGD |
| | | LGPRGERGQHGPKGEKGYPGIPPELQIAFMASLATH |
| | | FSNQNSGIIFSSVETNIGNFFDVMTGRFGAPVSGVYF |
| | | FTFSMMKHEDVEEVYVYLMHNGNTVFSMYSYEM |
| | | KGKSDTSSNHAVLKLAKGDEVWLRMGNGALHGD |
| | | HQRFSTFAGFLLFETK |
| 17 | Transmembrane domain | IFMYLLTVFLITQMIGSALFAVYLH |
| | of CD40L | |
| 18 | Transmembrane domain | MIETYNQTSPRSAATGLPISMKIFMYLLTVFLITQMI |
| | of CD40L | GSALFAVYLHRRLDK |
| 19 | Transmembrane domain | IYIWAPLAGICVALLLSLIITLICYH |
| | of CD8 | |
| 20 | Transmembrane domain | DIYIWAPLAGICVALLLSLIITLICYHR |
| | of CD8 | |
| 21 | Pilot peptide | YINL |
| 22 | Pilot peptide | YSHL |
| 23 | Pilot peptide | YINF |
| 24 | Pilot peptide | YSHF |
| 25 | Pilot peptide | DYINL |
| 26 | Pilot peptide | DYINF |
| 27 | Pilot peptide | PSAP |
| 28 | Pilot peptide | PTAP |
| 29 | Pilot peptide | PxxPxxxxPxxPxSxYxxLxPxxPExYxxLxPxxPDYxxL |
| 30 | Pilot peptide | PxxPxnPxxPxnSxYxxLxnPxxPExnYxxLxnPxxPDYxxL |
| 31 | Pilot peptide | PxxPxxxxPxxPxSxYxxLxPxxPExYxxLxPxxPDYxxLxxx |
| | | X |
| 32 | Pilot peptide | PxxPxnPxxPxnSxYxxLxnPxxPExnYxxLxnPxxPDYxxLx |
| | | XXX |
| 33 | Pilot peptide | PxxPxxxxxxxxxxxxYxxL |
| 34 | Pilot peptide | PxxPxxxxxxxxxxxDYxxL |
| 35 | Pilot peptide | PxxPxxYxxxxxxxxxYxxL |
| 36 | Pilot peptide | PxxPxxYxxxxxxxxDYxxL |
| 37 | Pilot peptide | PxxPExYxxLxPxxPDYxxL |
| 38 | Pilot peptide | PxxPxnYxxL |
| 39 | Pilot peptide | PxxPxnDYxxL |
| 40 | Pilot peptide | PxxPxnYxnYxxL |
| 41 | Pilot peptide | PxxPxnYxnDYxxL |
| 42 | Pilot peptide | PxxPExnYxxLxnPxxPDYxxL |
| 43 | Pilot peptide | PxxPxxxxPxxPxxxYxxLxPxxPExYxxLxPxxPDYxxL |
| 44 | Pilot peptide | PxxPxnPxxPxnYxxLxnPxxPExnYxxLxnPxxPDYxxL |
| 45 | Pilot peptide | PxxPxxxxPxxPxxxYxxLxPxxPExYxxLxPxxPDYxxLxxx |
| | | X |
| 46 | Pilot peptide | PxxPxnPxxPxnYxxLxnPxxPExnYxxLxnPxxPDYxxLxxx |
| | | X |
| 47 | Pilot peptide | APHFPEISFPPKPDSDYQALLPSAPEIYSHLSPTKPDY |
| | | INLRPAP |
| 48 | Linker | GGGS |
| 49 | Linker | GGGGS |
| 50 | Linker | GGGGGS |
| 51 | Linker | GGGSGGGGS |
| 52 | Linker | SGGGSGGGGSGGGSGGGGSGGGSGGGGSGGGGS |
| 53 | Linker | GGGSGGGGSGGGSGGGGSGGGSGGGGSGGGSG |
| 55 | Sub membrane targeting | (M)GXXKS |
| | domain | |
| 56 | Sub membrane targeting | CKXKXXXXRRR |
| | domain | |
| 57 | Sub membrane targeting | (M)GSSKSKPKDPSQRRR |
| | domain | |
| 58 | Sub membrane targeting | (M)GSSKSKPKDPSQRRRKSR |
| | domain | |
| 59 | Sub membrane targeting | (M)GSSKSKPKDPSQRRRKSRGPGG |
| | domain | |
| 60 | Sub membrane targeting | MGSSKSKPKDPSQRRRKSRGP |
| | domain | |
| 61 | Pilot peptide construct | MSRGGGAPHFPEISFPPKPDSDYQALLPSAPEIYSHL |
| | | SPTKPDYINLRPAPGGSRAT |
| 62 | Sub membrane targeting | MGSSKSKPKDPSQRRRKSRGPGGGSGGGSGGGSGG |
| | domain-linker construct | GSSRAT |
| 63 | Sub membrane targeting | MGSSKSKPKDPSQRRRKSRGPGGGSGGGSGGGSGG |
| | domain-linker-pilot | GSSRGGGAPHFPEISFPPKPDSDYQALLPSAPEIYSH |
| | peptide construct | LSPTKPDYINLRPAPGGSRAT |
| 64 | Linker-CD8 | SGGGSGGGGSGGGSGGGGSGGGSGGGGSGGGGSD |
| | transmembrane | IYIWAPLAGICVALLLSLIITLICYHRSRGAPHFPEISF |
| | domaine-pilot peptide | PPKPDSDYQALLPSAPEIYSHLSPTKPDYINLRPAP |
| | construct | |
| 65 | Pilot peptide-CD40L | MSRGGGAPHFPEISFPPKPDSDYQALLPSAPEIYSHL |
| | transmembrane domain- | SPTKPDYINLRPAPGGSRGSMIETYNQTSPRSAATG |
| | linker construct | LPISMKIFMYLLTVFLITQMIGSALFAVYLHRRLDK |
| | | GGGSGGGGSGGGSGGGGSGGGSGGGGSGGGSG |
| 66 | Sub membrane targeting | MGSSKSKPKDPSQRRRKSRGPGGGSGGGSGGGSGG |
| | domain-linker- | GSSRGSMIETYNQTSPRSAATGLPISMKIFMYLLTVF |
| | CD40Ltransmembrane | LITQMIGSALFAVYLHRRLDKGGGSGGGGSGGGSG |
| | domain-linker construct | GGGSGGGSGGGGSGGGSG |
| 67 | Sub membrane targeting | MGSSKSKPKDPSQRRRKSRGPGGGSGGGSGGGSGG |
| | domain-linker-pilot | GSSRGGGAPHFPEISFPPKPDSDYQALLPSAPEIYSH |
| | peptide-CD40L | LSPTKPDYINLRPAPGGSRGSMIETYNQTSPRSAAT |
| | transmembrane domain- | GLPISMKIFMYLLTVFLITQMIGSALFAVYLHRRLD |
| | linker construct | KGGGSGGGGSGGGSGGGGSGGGSGGGGSGGGSG |
| 68 | Neuraminidase | MSRGGGAPHFPEISFPPKPDSDYQALLPSAPEIYSHL |
| | construct | SPTKPDYINLRPAPGGSRATMNPNQKIITIGSVSLTIS |
| | | TICFFMQIAILITTVTLHFKQYEFNSPPNNQVMLCEP |
| | | TIIERNITEIVYLTNTTIEKEICPKPAEYRNWSKPQCD |
| | | ITGFAPFSKDNSIRLSAGGDIWVTREPYVSCDPDKC |
| | | YQFALGQGTTLNNVHSNNTVRDRTPYRTLLMNEL |
| | | GVPFHLGTKQVCIAWSSSSCHDGKAWLHVCITGDD |
| | | KNATASFIYNGRLVDSVVSWSKEILRTQESECVCIN |
| | | GTCTVVMTDGSASGKADTKILFIEEGKVVHTSTLSG |
| | | SAQHVEECSCYPRYPGVRCVCRDNWKGSNRPIVDI |
| | | NIKDHSIVSSYVCSGLVGDTPRKNDSSSSSHCLDPN |
| | | NEEGGHGVKGWAFDDGNDVWMGRTINETSRLGY |
| | | ETFKVIEGWSNPKSKLQINRQVIVDRGDRSGYSGIFS |
| | | VEGKSCINRCFYVELIRGRKEETEVLWTSNSIVVFC |
| | | GTSGTYGTGSWPDGADLNLMPI |
| 69 | TRAIL construct | MSRGGGAPHFPEISFPPKPDSDYQALLPSAPEIYSHL |
| | | SPTKPDYINLRPAPGGSRATMAMMEVQGGPSLGQT |
| | | CVLIVIFTVLLQSLCVAVTYVYFTNELKQMQDKYS |
| | | KSGIACFLKEDDSYWDPNDEESMNSPCWQVKWQL |
| | | RQLVRKMILRTSEETISTVQEKQQNISPLVRERGPQR |
| | | VAAHITGTRGRSNTLSSPNSKNEKALGRKINSWESS |
| | | RSGHSFLSNLHLRNGELVIHEKGFYYIYSQTYFRFQ |
| | | EEIKENTKNDKQMVQYIYKYTSYPDPILLMKSARNS |
| | | CWSKDAEYGLYSIYQGGIFELKENDRIFVSVTNEHL |
| | | IDMDHEASFFGAFLVG |
| 70 | TNFa construct | MSRGGGAPHFPEISFPPKPDSDYQALLPSAPEIYSHL |
| | | SPTKPDYINLRPAPGGSRATMSTESMIRDVELAEEA |
| | | LPKKTGGPQGSRRCLFLSLFSFLIVAGATTLFCLLHF |
| | | GVIGPQREEFPRDLSLISPLAQAVRSSSRTPSDKPVA |
| | | HVVANPQAEGQLQWLNRRANALLANGVELRDNQ |
| | | LVVPSEGLYLIYSQVLFKGQGCPSTHVLLTHTISRIA |
| | | VSYQTKVNLLSAIKSPCQRETPEGAEAKPWYEPIYL |
| | | GGVFQLEKGDRLSAEINRPDYLDFAESGQVYFGIIA |
| | | L |
| 71 | CD30L construct | MSRGGGAPHFPEISFPPKPDSDYQALLPSAPEIYSHL |
| | | SPTKPDYINLRPAPGGSRATMDPGLQQALNGMAPP |
| | | GDTAMHVPAGSVASHLGTTSRSYFYLTTATLALCL |
| | | VFTVATIMVLVVQRTDSIPNSPDNVPLKGGNCSEDL |
| | | LCILKRAPFKKSWAYLQVAKHLNKTKLSWNKDGIL |
| | | HGVRYQDGNLVIQFPGLYFIICQLQFLVQCPNNSVD |
| | | LKLELLINKHIKKQALVTVCESGMQTKHVYQNLSQ |
| | | FLLDYLQVNTTISVNVDTFQYIDTSTFPLENVLSIFL |
| | | YSNSD |
| 72 | C137L construct 1 | MSRGGGAPHFPEISFPPKPDSDYQALLPSAPEIYSHL |
| | | SPTKPDYINLRPAPGGSRATMEYASDASLDPEAPWP |
| | | PAPRARACRVLPWALVAGLLLLLLLAAACAVFLAC |
| | | PWAVSGARASPGSAASPRLREGPELSPDDPAGLLDL |
| | | RQGMFAQLVAQNVLLIDGPLSWYSDPGLAGVSLTG |
| | | GLSYKEDTKELVVAKAGVYYVFFQLELRRVVAGE |
| | | GSGSVSLALHLQPLRSAAGAAALALTVDLPPASSEA |
| | | RNSAFGFQGRLLHLSAGQRLGVHLHTEARARHAW |
| | | QLTQGATVLGLFRVTPEIPAGLPSPRSE |
| 73 | CD137L construct 2 | MGSSKSKPKDPSQRRRKSRGPGGGSGGGSGGGSGG |
| | | GSSRATMEYASDASLDPEAPWPPAPRARACRVLPW |
| | | ALVAGLLLLLLLAAACAVFLACPWAVSGARASPGS |
| | | AASPRLREGPELSPDDPAGLLDLRQGMFAQLVAQN |
| | | VLLIDGPLSWYSDPGLAGVSLTGGLSYKEDTKELV |
| | | VAKAGVYYVFFQLELRRVVAGEGSGSVSLALHLQP |
| | | LRSAAGAAALALTVDLPPASSEARNSAFGFQGRLL |
| | | HLSAGQRLGVHLHTEARARHAWQLTQGATVLGLF |
| | | RVTPEIPAGLPSPRSE |
| 74 | CD137L construct 3 | MGSSKSKPKDPSQRRRKSRGPGGGSGGGSGGGSGG |
| | | GSSRGGGAPHFPEISFPPKPDSDYQALLPSAPEIYSH |
| | | LSPTKPDYINLRPAPGGSRATMEYASDASLDPEAPW |
| | | PPAPRARACRVLPWALVAGLLLLLLLAAACAVFLA |
| | | CPWAVSGARASPGSAASPRLREGPELSPDDPAGLL |
| | | DLRQGMFAQLVAQNVLLIDGPLSWYSDPGLAGVSL |
| | | TGGLSYKEDTKELVVAKAGVYYVFFQLELRRVVA |
| | | GEGSGSVSLALHLQPLRSAAGAAALALTVDLPPASS |
| | | EARNSAFGFQGRLLHLSAGQRLGVHLHTEARARHA |
| | | WQLTQGATVLGLFRVTPEIPAGLPSPRSE |
| 75 | CTRP3 construct 1 | QDEYMESPQTGGLPPDCSKCCHGDYSFRGYQGPPG |
| | | PPGPPGIPGNHGNNGNNGATGHEGAKGEKGDKGD |
| | | LGPRGERGQHGPKGEKGYPGIPPELQIAFMASLATH |
| | | FSNQNSGIIFSSVETNIGNFFDVMTGRFGAPVSGVYF |
| | | FTFSMMKHEDVEEVYVYLMHNGNTVFSMYSYEM |
| | | KGKSDTSSNHAVLKLAKGDEVWLRMGNGALHGD |
| | | HQRFSTFAGFLLFETKSGGGSGGGGSGGGSGGGGS |
| | | GGGSGGGGSGGGGSDIYIWAPLAGICVALLLSLIITL |
| | | ICYHRSRGAPHFPEISFPPKPDSDYQALLPSAPEIYSH |
| | | LSPTKPDYINLRPAP |
| 76 | TRAIL external domain | MSRGGGAPHFPEISFPPKPDSDYQALLPSAPEIYSHL |
| | construct | SPTKPDYINLRPAPGGSRGSMIETYNQTSPRSAATG |
| | | LPISMKIFMYLLTVFLITQMIGSALFAVYLHRRLDK |
| | | GGGSGGGGSGGGSGGGGSGGGSGGGGSGGGSGTN |
| | | ELKQMQDKYSKSGIACFLKEDDSYWDPNDEESMN |
| | | SPCWQVKWQLRQLVRKMILRTSEETISTVQEKQQN |
| | | ISPLVRERGPQRVAAHITGTRGRSNTLSSPNSKNEK |
| | | ALGRKINSWESSRSGHSFLSNLHLRNGELVIHEKGF |
| | | YYIYSQTYFRFQEEIKENTKNDKQMVQYIYKYTSYP |
| | | DPILLMKSARNSCWSKDAEYGLYSIYQGGIFELKEN |
| | | DRIFVSVTNEHLIDMDHEASFFGAFLVG |
| 77 | TRAIL TNF-like | MSRGGGAPHFPEISFPPKPDSDYQALLPSAPEIYSHL |
| | domain construct | SPTKPDYINLRPAPGGSRGSMIETYNQTSPRSAATG |
| | | LPISMKIFMYLLTVFLITQMIGSALFAVYLHRRLDK |
| | | GGGSGGGGSGGGSGGGGSGGGSGGGGSGGGSGSW |
| | | ESSRSGHSFLSNLHLRNGELVIHEKGFYYIYSQTYFR |
| | | FQEEIKENTKNDKQMVQYIYKYTSYPDPILLMKSAR |
| | | NSCWSKDAEYGLYSIYQGGIFELKENDRIFVSVTNE |
| | | HLIDMDHEASFFGAFLVG |
| 78 | TNFa external domain | MSRGGGAPHFPEISFPPKPDSDYQALLPSAPEIYSHL |
| | construct | SPTKPDYINLRPAPGGSRGSMIETYNQTSPRSAATG |
| | | LPISMKIFMYLLTVFLITQMIGSALFAVYLHRRLDK |
| | | GGGSGGGGSGGGSGGGGSGGGSGGGGSGGGSGGP |
| | | QREEFPRDLSLISPLAQAVRSSSRTPSDKPVAHVVA |
| | | NPQAEGQLQWLNRRANALLANGVELRDNQLVVPS |
| | | EGLYLIYSQVLFKGQGCPSTHVLLTHTISRIAVSYQT |
| | | KVNLLSAIKSPCQRETPEGAEAKPWYEPIYLGGVFQ |
| | | LEKGDRLSAEINRPDYLDFAESGQVYFGIIAL |
| 79 | TNFa mature domain | MSRGGGAPHFPEISFPPKPDSDYQALLPSAPEIYSHL |
| | construct | SPTKPDYINLRPAPGGSRGSMIETYNQTSPRSAATG |
| | | LPISMKIFMYLLTVFLITQMIGSALFAVYLHRRLDK |
| | | GGGSGGGGSGGGSGGGGSGGGSGGGGSGGGSGLQ |
| | | WLNRRANALLANGVELRDNQLVVPSEGLYLIYSQV |
| | | LFKGQGCPSTHVLLTHTISRIAVSYQTKVNLLSAIKS |
| | | PCQRETPEGAEAKPWYEPIYLGGVFQLEKGDRLSA |
| | | EINRPDYLDFAESGQVYFGIIAL |
| 80 | CD30L external domain | MSRGGGAPHFPEISFPPKPDSDYQALLPSAPEIYSHL |
| | construct | SPTKPDYINLRPAPGGSRGSMIETYNQTSPRSAATG |
| | | LPISMKIFMYLLTVFLITQMIGSALFAVYLHRRLDK |
| | | GGGSGGGGSGGGSGGGGSGGGSGGGGSGGGSGQR |
| | | TDSIPNSPDNVPLKGGNCSEDLLCILKRAPFKKSWA |
| | | YLQVAKHLNKTKLSWNKDGILHGVRYQDGNLVIQ |
| | | FPGLYFIICQLQFLVQCPNNSVDLKLELLINKHIKKQ |
| | | ALVTVCESGMQTKHVYQNLSQFLLDYLQVNTTISV |
| | | NVDTFQYIDTSTFPLENVLSIFLYSNSD |
| 81 | TGF-β1 construct | MSRGGGAPHFPEISFPPKPDSDYQALLPSAPEIYSHL |
| | | SPTKPDYINLRPAPGGSRGSMIETYNQTSPRSAATG |
| | | LPISMKIFMYLLTVFLITQMIGSALFAVYLHRRLDK |
| | | GGGSGGGGSGGGSGGGGSGGGSGGGGSGGGSGLS |
| | | TCKTIDMELVKRKRIEAIRGQILSKLRLASPPSQGEV |
| | | PPGPLPEAVLALYNSTRDRVAGESAEPEPEPEADYY |
| | | AKEVTRVLMVETHNEIYDKFKQSTHSIYMFFNTSEL |
| | | REAVPEPVLLSRAELRLLRLKLKVEQHVELYQKYS |
| | | NNSWRYLSNRLLAPSDSPEWLSFDVTGVVRQWLSR |
| | | GGEIEGFRLSAHCSCDSRDNTLQVDINGFTTGRRGD |
| | | LATIHGMNRPFLLLMATPLERAQHLQSSRHRRALD |
| | | TNYCFSSTEKNCCVRQLYIDFRKDLGWKWIHEPKG |
| | | YHANFCLGPCPYIWSLDTQYSKVLALYNQHNPGAS |
| | | AAPCCVPQALEPLPIVYYVGRKPKVEQLSNMIVRSC |
| | | KCS |
| 82 | BMP7 construct | MSRGGGAPHFPEISFPPKPDSDYQALLPSAPEIYSHL |
| | | SPTKPDYINLRPAPGGSRGSMIETYNQTSPRSAATG |
| | | LPISMKIFMYLLTVFLITQMIGSALFAVYLHRRLDK |
| | | GGGSGGGGSGGGSGGGGSGGGSGGGGSGGGSGDF |
| | | SLDNEVHSSFIHRRLRSQERREMQREILSILGLPHRP |
| | | RPHLQGKHNSAPMFMLDLYNAMAVEEGGGPGGQ |
| | | GFSYPYKAVFSTQGPPLASLQDSHFLTDADMVMSF |
| | | VNLVEHDKEFFHPRYHHREFRFDLSKIPEGEAVTAA |
| | | EFRIYKDYIRERFDNETFRISVYQVLQEHLGRESDLF |
| | | LLDSRTLWASEEGWLVFDITATSNHWVVNPRHNLG |
| | | LQLSVETLDGQSINPKLAGLIGRHGPQNKQPFMVAF |
| | | FKATEVHFRSIRSTGSKQRSQNRSKTPKNQEALRM |
| | | ANVAENSSSDQRQACKKHELYVSFRDLGWQDWIIA |
| | | PEGYAAYYCEGECAFPLNSYMNATNHAIVQTLVHF |
| | | INPETVPKPCCAPTQLNAISVLYFDDSSNVILKKYRN |
| | | MVVRACGCH |
| 83 | BMP9 construct | MSRGGGAPHFPEISFPPKPDSDYQALLPSAPEIYSHL |
| | | SPTKPDYINLRPAPGGSRGSMIETYNQTSPRSAATG |
| | | LPISMKIFMYLLTVFLITQMIGSALFAVYLHRRLDK |
| | | GGGSGGGGSGGGSGGGGSGGGSGGGGSGGGSGKP |
| | | LQSWGRGSAGGNAHSPLGVPGGGLPEHTFNLKMF |
| | | LENVKVDFLRSLNLSGVPSQDKTRVEPPQYMIDLY |
| | | NRYTSDKSTTPASNIVRSFSMEDAISITATEDFPFQK |
| | | HILLFNISIPRHEQITRAELRLYVSCQNHVDPSHDLK |
| | | GSVVIYDVLDGTDAWDSATETKTFLVSQDIQDEGW |
| | | ETLEVSSAVKRWVRSDSTKSKNKLEVTVESHRKGC |
| | | DTLDISVPPGSRNLPFFVVFSNDHSSGTKETRLELRE |
| | | MISHEQESVLKKLSKDGSTEAGESSHEEDTDGHVA |
| | | AGSTLARRKRSAGAGSHCQKTSLRVNFEDIGWDS |
| | | WIIAPKEYEAYECKGGCFFPLADDVTPTKHAIVQTL |
| | | VHLKFPTKVGKACCVPTKLSPISVLYKDDMGVPTL |
| | | KYHYEGMSVAECGCR |
| 84 | GDNF construct | MSRGGGAPHFPEISFPPKPDSDYQALLPSAPEIYSHL |
| | | SPTKPDYINLRPAPGGSRGSMIETYNQTSPRSAATG |
| | | LPISMKIFMYLLTVFLITQMIGSALFAVYLHRRLDK |
| | | GGGSGGGGSGGGSGGGGSGGGSGGGGSGGGSGFP |
| | | LPAGKRPPEAPAEDRSLGRRRAPFALSSDSNMPEDY |
| | | PDQFDDVMDFIQATIKRLKRSPDKQMAVLPRRERN |
| | | RQAAAANPENSRGKGRRGQRGKNRGCVLTAIHLN |
| | | VTDLGLGYETKEELIFRYCSGSCDAAETTYDKILKN |
| | | LSRNRRLVSDKVGQACCRPIAFDDDLSFLDDNLVY |
| | | HILRKHSAKRCGCI |
| 85 | BDNF construct | MSRGGGAPHFPEISFPPKPDSDYQALLPSAPEIYSHL |
| | | SPTKPDYINLRPAPGGSRGSMIETYNQTSPRSAATG |
| | | LPISMKIFMYLLTVFLITQMIGSALFAVYLHRRLDK |
| | | GGGSGGGGSGGGSGGGGSGGGSGGGGSGGGSGAP |
| | | MKEANIRGQGGLAYPGVRTHGTLESVNGPKAGSR |
| | | GLTSLADTFEHVIEELLDEDQKVRPNEENNKDADL |
| | | YTSRVMLSSQVPLEPPLLFLLEEYKNYLDAANMSM |
| | | RVRRHSDPARRGELSVCDSISEWVTAADKKTAVD |
| | | MSGGTVTVLEKVPVSKGQLKQYFYETKCNPMGYT |
| | | KEGCRGIDKRHWNSQCRTTQSYVRALTMDSKKRIG |
| | | WRFIRIDTSCVCTLTIKRGR |
| 86 | CTRP3 construct 2 | MSRGGGAPHFPEISFPPKPDSDYQALLPSAPEIYSHL |
| | | SPTKPDYINLRPAPGGSRGSMIETYNQTSPRSAATG |
| | | LPISMKIFMYLLTVFLITQMIGSALFAVYLHRRLDK |
| | | GGGSGGGGSGGGSGGGGSGGGSGGGGSGGGSGQD |
| | | EYMESPQTGGLPPDCSKCCHGDYSFRGYQGPPGPP |
| | | GPPGIPGNHGNNGNNGATGHEGAKGEKGDKGDLG |
| | | PRGERGQHGPKGEKGYPGIPPELQIAFMASLATHES |
| | | NQNSGIIFSSVETNIGNFFDVMTGRFGAPVSGVYFFT |
| | | FSMMKHEDVEEVYVYLMHNGNTVFSMYSYEMKG |
| | | KSDTSSNHAVLKLAKGDEVWLRMGNGALHGDHQ |
| | | RFSTFAGFLLFETK |
| 87 | CTRP3 construct 3 | MGSSKSKPKDPSQRRRKSRGPGGGSGGGSGGGSGG |
| | | GSSRGSMIETYNQTSPRSAATGLPISMKIFMYLLTVF |
| | | LITQMIGSALFAVYLHRRLDKGGGSGGGGSGGGSG |
| | | GGGSGGGSGGGGSGGGSGQDEYMESPQTGGLPPD |
| | | CSKCCHGDYSFRGYQGPPGPPGPPGIPGNHGNNGN |
| | | NGATGHEGAKGEKGDKGDLGPRGERGQHGPKGEK |
| | | GYPGIPPELQIAFMASLATHFSNQNSGIIFSSVETNIG |
| | | NFFDVMTGRFGAPVSGVYFFTFSMMKHEDVEEVY |
| | | VYLMHNGNTVFSMYSYEMKGKSDTSSNHAVLKLA |
| | | KGDEVWLRMGNGALHGDHQRFSTFAGFLLFETK |
| 88 | CTRP3 construct 4 | MGSSKSKPKDPSQRRRKSRGPGGGSGGGSGGGSGG |
| | | GSSRGGGAPHFPEISFPPKPDSDYQALLPSAPEIYSH |
| | | LSPTKPDYINLRPAPGGSRGSMIETYNQTSPRSAAT |
| | | GLPISMKIFMYLLTVFLITQMIGSALFAVYLHRRLD |
| | | KGGGSGGGGSGGGSGGGGSGGGSGGGGSGGGSGQ |
| | | DEYMESPQTGGLPPDCSKCCHGDYSFRGYQGPPGP |
| | | PGPPGIPGNHGNNGNNGATGHEGAKGEKGDKGDL |
| | | GPRGERGQHGPKGEKGYPGIPPELQIAFMASLATHF |
| | | SNQNSGIIFSSVETNIGNFFDVMTGRFGAPVSGVYFF |
| | | TFSMMKHEDVEEVYVYLMHNGNTVFSMYSYEMK |
| | | GKSDTSSNHAVLKLAKGDEVWLRMGNGALHGDH |
| | | QRFSTFAGFLLFETK |
| 89 | Proline-rich motif | PPPP |
| 90 | OX40L | MERVQPLEENVGNAARPRFERNKLLLVASVIQGLG |
| | | LLLCFTYICLHFSALQVSHRYPRIQSIKVQFTEYKKE |
| | | KGFILTSQKEDEIMKVQNNSVIINCDGFYLISLKGYF |
| | | SQEVNISLHYQKDEEPLFQLKKVRSVNSLMVASLT |
| | | YKDKVYLNVTTDNTSLDDFHVNGGELILIHQNPGE |
| | | FCVL |
| 91 | OX40L construct | MSRGGGAPHFPEISFPPKPDSDYQALLPSAPEIYSHL |
| | | SPTKPDYINLRPAPGGSRATMERVQPLEENVGNAA |
| | | RPRFERNKLLLVASVIQGLGLLLCFTYICLHFSALQV |
| | | SHRYPRIQSIKVQFTEYKKEKGFILTSQKEDEIMKVQ |
| | | NNSVIINCDGFYLISLKGYFSQEVNISLHYQKDEEPL |
| | | FQLKKVRSVNSLMVASLTYKDKVYLNVTTDNTSL |
| | | DDFHVNGGELILIHQNPGEFCVL |

### EXAMPLES

The present invention is further illustrated by the following examples.

### Example 1: Production of Semi-Purified Extracellular Vesicles Comprising a Chimeric Protein of Interest

### Materials and Methods

### Production of Protein of Interest and of Extracellular Vesicles Harboring the Protein of Interest in Mammalian Cells

Extracellular vesicles were produced in HEK293T cells, obtained from American Type Culture Collection (ATCC). Cells were cultured in DMEM supplemented with 5% of heat-inactivated fetal bovine serum (iFBS), 2 mM of GlutaMAX and 5 μg/mL of gentamicin at 37° C. in a 5% CO₂ humidified incubator. HEK293T cells were routinely tested and found negative by MycoAlert™ mycoplasma detection kit (Lonza Nottingham, Ltd.).

Nucleic acid sequences coding for chimeric proteins of interest targeted to exosomes (SEQ ID Nos: 68-88) were inserted in an eucaryotic expression vector under the control of a CMV/HTLV chimeric promoter. When necessary, a zeocin encoding resistance gene was added in tandem with the protein of interest-coding nucleic acid sequence and downstream a CMV IRES sequence, allowing for simultaneous expression of zeocin resistance and establishment of stable transfected cell line. These nucleic acid sequences were transfected into HEK293T cells using PEI. When necessary, the selection of a stable transfected cell line was obtained in the presence of 500 μg/mL of zeocin during 15 days.

In order to generate large-scale exosome production, HEK293T transfected cells were plated into cell chambers of 10 trays in 1 L of complete medium. 24 hours later, cultures were fed with medium supplemented with extracellular vesicle-free iFBS and incubated for a further 48 hours.

### Protein of Interest-Extracellular Vesicles and Extracellular Vesicle Purification

Cell culture medium was harvested from transfected HEK293T cells and protein of interest-extracellular vesicle isolation was performed as previously described (Taylor & Shah, 2015. Methods. 87:3-10; Desplantes et al., 2017. Sci Rep. 7(1):1032; Corso G. et al. 2017. Scientific Reports. 7:11561. DOI: 10.1038/s41598-017-10646-x). Briefly, cell culture supernatant was clarified by two consecutive centrifugations: 10 minutes at 1 300 rpm and 15 minutes at 4 000 rpm, both at 4° C., followed by filtration through 0.22 μm membrane filters. The supernatant was then concentrated by ultra-filtration and diafiltration and load onto either size exclusion chromatography (SEC) or BE-SEC columns (CL2-B or Sephacryl S1000 or Captocore, GE Healthcare). Fractions containing extracellular vesicle biomarkers (CD81 and CD63) were identified by ELISA. Extracellular vesicle fractions containing protein of interest identified by Western-Blot were concentrated when necessary and used for analysis and injections.

### SDS-PAGE, Western-Blotting and Antibodies

Protein concentration of protein of interest-extracellular vesicles was measured using the BCA assay (Pierce BCA Protein Assay Kit, ThermoFisher Scientific). Protein of interest-extracellular vesicles preparations were lysed and separated by SDS-PAGE on a 4-15% acrylamide gel (4-15% Mini-PROTEAN® TGX Stain-Free™ Gel kit, Bio-Rad) and subsequently transferred onto PVDF membrane. For Western-Blotting in non-reducing conditions, a loading buffer without DTT was used.

Immunodetection of protein of interest was carried out with primary antibodies against either the protein of interest, or anti-Ciloa Pilot Peptide (PP) (in-house antibody raised in rabbit).

Immunodetection of specific extracellular vesicles markers was carried out with primary antibodies against either CD81 (Genetex Ref. #GTX101766), CD63 (Genetex Ref. #GTX132953), Alix (Proteintech #12422-1-AP), syntenin (Fisher Scientific Ref. #11326573).

Membranes were then incubated with the corresponding secondary HRP-conjugated antibodies (donkey anti-mouse or anti-rabbit or anti-goat HRP, Jackson ImmunoResearch, Refs. #715-035-150, #711-035-152 or #715-038-147).

The signals were detected using an enhanced chemiluminescence detection kit (Super Signal West Pico Plus; ThermoFischer Scientific; Ref. 34580) and membranes imaged with ChemiDoc Imaging System (Bio-Rad).

These primary antibodies, as well as respective secondary antibodies, were also used to detect the protein of interest on the surface of protein of interest-extracellular vesicles by ELISA.

### Protein of Interest and Extracellular Vesicle Marker Specific IgG ELISA

Extracellular vesicle surface contents in protein of interest, and in CD81- and CD63-specific surface markers were determined by ELISA using some of the above antibodies, and also anti-CD81 (Ancell; Ref. #ANC-302-020) or anti-CD63 (Agro-Bio; Ref. #S12086) antibodies.

Briefly, MaxiSorp ELISA plates (Nunc) were coated with serial 1/2 dilutions (starting from 1 μg) protein of interest-extracellular vesicles in 100 μL in 50 mM sodium carbonate/bicarbonate pH 9.6 buffer per well, overnight at 4° C. Coated plates were washed 3 times with 200 μL of 1×PBS and saturated for 1 hour at 37° C. with 200 μL of 3% BSA in 1×PBS per well. Plates were washed three times with 1×PBS, then incubated in 3% BSA and 5% FBS with primary antibody dilutions (1:500 for adiponectin or 1:10000 for extracellular vesicle-specific markers) for 2 hours at 37° C.

This was followed by 3 washes with 200 μL of 1×PBS per well and incubation with 100 μL per well of corresponding secondary HRP conjugated antibody (as specified for Western-Blots above) diluted 1:10000 in 3% BSA in 1×PBS. Following incubation with the secondary antibody, plates were washed 5 times with 200 μL of 1×PBS per well and developed with 100 μL of TMB per well (Bio-Rad; Ref. #R8/R9) for 30 minutes. The reaction was stopped by adding 50 μL of stop solution (2 N sulfuric acid) per well.

The 450 nm-absorbance was read using ClarioStar Plus plate reader (BMG Labtech). The reciprocal endpoint titers were defined as the dilution with the 450 nm OD 3 times higher than the background.

### Example 2: Production of Ultra-Purified Extracellular Vesicles Comprising a Chimeric Protein of Interest

### Materials and Methods

### Production of Protein of Interest and of Extracellular Vesicles Harboring Protein of Interest in Mammalian Cells

Extracellular vesicles harbouring chimeric protein of interest (SEQ ID Nos: 68-88) were produced as described hereinabove.

Production of Protein of Interest on EVs Ultra-Purified from Culture Medium

Culture medium of cells stably expressing DNA constructs comprising different proteins of interest (SEQ ID Nos: 68-88), or of control cells was concentrated and purified using TFF and BE-SEC chromatography. The ultra-purified EVs as well as extracts from producer cells were subjected to SDS-PAGE separation in reducing or non-reducing conditions, analyzed by Western-blot and revealed with anti-protein of interest primary antibody followed by a secondary HRP-conjugated antibody, as described hereinabove.

### Characterization of Ultra-Purified EVs

Culture medium of cells stably expressing DNA constructs comprising different proteins of interest (SEQ ID Nos: 68-88), or of control cells was concentrated and purified using TFF and BE-SEC chromatography. The ultra-purified EVs as well as extracts from producer cells were subjected to SDS-PAGE separation in reducing or non-reducing conditions, analyzed by Western-blot and revealed with anti-Alix (EV marker) and anti-protein of interest primary antibodies followed by a secondary HRP-conjugated antibody.

The presence of an EV marker (CD81) and of the protein of interest on the surface of EVs was detected by ELISA. The different types of EVs were fixed on ELISA plates in dilutions from 1 to 1/128 (where 1=50 μl of pure EVs) and detected by anti-CD81 antibody or anti-protein of interest antibody followed by a secondary anti-HRP antibody.

### Example 3: Expression of the Chimeric Polypeptides

### Materials and Methods

### Cell Culture and Extracellular Vesicle Production

HEK293T cells were cultured in Dulbecco's modified Eagle's medium (DMEM) supplemented with 5% heat inactivated fetal bovine serum (iFBS), 2 mM GlutaMAX and 5 μg/mL gentamicin at 37° C. in a 5% CO₂ humidified incubator.

HEK293T cells, plated in culture flasks in complete medium, were transfected with DNA encoding the different protein constructs using Polyethyleneimine. Zeocine (Invivogen) selection pressure was applied in order to establish stable cell lines. Mycoplasma absence was assessed using the MycoStrip™ kit (Invivogen). For EV production, cells were cultured 48 h in either T150 Flask or 10 layers cell-factories in an exosome-free medium.

### Extracellular Vesicle Purification

Conditioned cell culture medium was harvested and was clarified by two consecutive centrifugations: 10 min at 300 g and 15 min at 3,000 g, both at 4° C. Clarified culture medium was filtrated on 0.22 μm membrane filters and then submitted either i) for small volume to ultracentrifugation at average 110,000 g for 2 hours (TLA100.1 rotor, Beckman Coulter) followed by the exosome (EV) pellet solubilization directly in Laemmli sample buffer for Western blot (WB) analysis; or ii) for large volume, to filtration, diafiltration and concentration by TFF; concentrated EVs were finally purified by a chromatography based on size (SEC); finally, fractions containing pure EVs were pooled, EVs were sterile filtrated on 0.22 μm filters and stored at 4° C. EVs purified using this later protocol, can be submitted to both WB analysis or ELISA.

### Extracellular Vesicle Size Distribution, Particle Numbering

EV size distribution and particle number were obtained by nanoflow cytometry using a NanoAnalyzer instrument (NanoFCM).

### SDS-PAGE, Western Blotting (WB) and Antibodies

To prepare cell extracts, cells were harvested and centrifuged at 300 g for 10 min at 4° C., washed in 1X Phosphate Buffered Saline (PBS) and lysed in 1% IGEPAL (Sigma).

Protein concentrations of cell extracts and EV batches were determined using the BCA assay (Thermo Scientific). For SDS-PAGE, 5 μg of pure EVs or 10 μg of cell extracts were lysed in Laemmli sample buffer and heated for 5 min at 95° C. EV and cell extract preparations were separated by SDS-PAGE on a 4-15% gradient polyacrylamide gel (Bio-Rad) and proteins were subsequently transferred onto PVDF membrane. The immunodetection of proteins was performed with primary antibodies recognizing specifically either Alix (mouse monoclonal #12422-1-AP, Proteintech), CD137L (mouse monoclonal #GTX117355, GeneTex) or the pilot peptide (rabbit, in-house antibody). Membranes were then incubated with the corresponding secondary Horseradish Peroxidase (HRP)-conjugated antibodies (donkey anti-mouse HRP or donkey anti-rabbit HRP, Jackson ImmunoResearch). The signals were detected using an enhanced chemiluminescence detection kit (Thermo Scientific) and membranes were imaged with a Chemidoc Imaging System (Bio-Rad).

### Anti-CD81 ELISA

Serial dilutions of pure EVs (from 1 μg to 1 ng) were coated onto a 96-well ELISA plate overnight at 4° C. After saturation with 3% BSA in PBS during 1 h at 37° C., anti-CD81 antibody was added and incubated for 2 h at 37° C. Then, the plate was washed three times and incubated with secondary HRP-conjugated anti-mouse IgG (donkey antibody, Jackson ImmunoResearch) for 1 h at 37° C. After washing 5 times, 3, 3′, 5, 5′-Tetramethylbenzidine (TMB), the chromogenic peroxidase substrate, was added and the plate was incubated in the dark for 30 min at room temperature. Sulfuric acid was added to stop the reaction. Optical density (OD) was measured at 450 nm using a CLARIOstar Plus plate reader (BMG Labtech).

### Results

### CD137L Construct Expression

HEK293T cells were transfected with DNA encoding different protein constructs: (i) DNA encoding wild-type CD137L protein (encoding the polypeptide with SEQ ID NO: 10), (ii) DNA encoding CD137L construct 1 comprising a pilot peptide fused upstream of CD137L (encoding the polypeptide with SEQ ID NO: 72), (iii) DNA encoding CD137L construct 2 comprising a Src peptide fused upstream of CD137L (encoding the polypeptide with SEQ ID NO: 73), or (iv) DNA encoding 137L construct 3 comprising both Src peptide fused and pilot peptide fused upstream of CD137L (encoding the polypeptide with SEQ ID NO: 74). Extracellular vesicles produced by cells transfected by each of the DNA construct and also by a control DNA were purified, lysed and their protein contents were separated by polyacrylamide gel electrophoresis and analyzed by immunotransfert (WB).

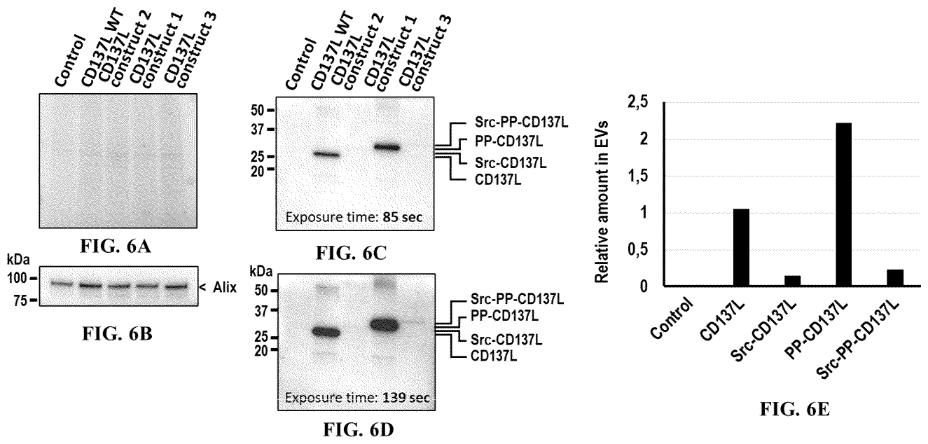

FIG. 6A represents the proteins of each extracellular vesicle lysates analyzed using Mini-Protean TGX Stain Free Gels, 4-15% (BioRad), and indicates that similar amounts of extracellular vesicles were loaded in each gel lane.

FIG. 6B represents a western blot probed with anti-Alix antibodies (i.e., Alix is an extracellular vesicle specific marker). The similar intensities of Alix bands in each lane confirms that similar amounts of extracellular vesicles were loaded in each gel lane.

FIGS. 6C-D represent the same western blot probed with anti-CD137L antibodies. In FIG. 6C, the exposure time was 85 sec. In order to visualize the faint bands corresponding to Src-CD137L construct 2 (SEQ ID NO: 73) and Src-PP-CD137L construct 3 (SEQ ID NO: 74), FIG. 6D presents a higher exposure time of 139 sec. Bands are present at the apparent molecular weights of 27 kDa, 30 kDa, 33 kDa, and 36 kDa corresponding to the expected molecular weight for wild-type CD137L protein (SEQ ID NO: 10), Src-CD137L construct 2 (SEQ ID NO: 73), PP-CD137L construct 1 (SEQ ID NO: 72), and Src-PP-CD137L construct 3 (SEQ ID NO: 74), respectively.

FIG. 6E shows a histogram with the amount of each CD137L construct relative to Alix amount (ratio of amount of pixels in CD137L construct bands/amount of pixels in Alix band) in each lane of the presented WBs. As shown on FIG. 6E, some wild-type CD137L protein (SEQ ID NO: 10) is spontaneously targeted to extracellular vesicles. CD137L construct 1 (SEQ ID NO: 72), corresponding to a pilot peptide fused upstream of CD137L exhibits an increased targeting to extracellular vesicles, as compared to the wild-type CD137L protein. On the opposite, CD137L construct 2 (SEQ ID NO: 73) corresponding to a Src peptide fused upstream of CD137L, and CD137L construct 3 (SEQ ID NO: 74) corresponding to both Src peptide and pilot peptide fused upstream of CD137L, exhibit a strongly decreased targeting to extracellular vesicles as compared to wild-type CD137L protein.

These results show that the pilot peptide increases the amount of CD137L targeting to extracellular vesicles, while the addition of the Src peptide inhibit CD137L targeting to extracellular vesicles.

### Characterization of Extracellular Vesicles Harboring CD137L Protein

HEK293T cells were transfected with DNA encoding CD137L construct 1 comprising a pilot peptide fused upstream of CD137L (encoding the polypeptide with SEQ ID NO: 72). Extracellular vesicles produced by transfected cells were purified and analyzed by nanoflow cytometry and ELISA for CD81.

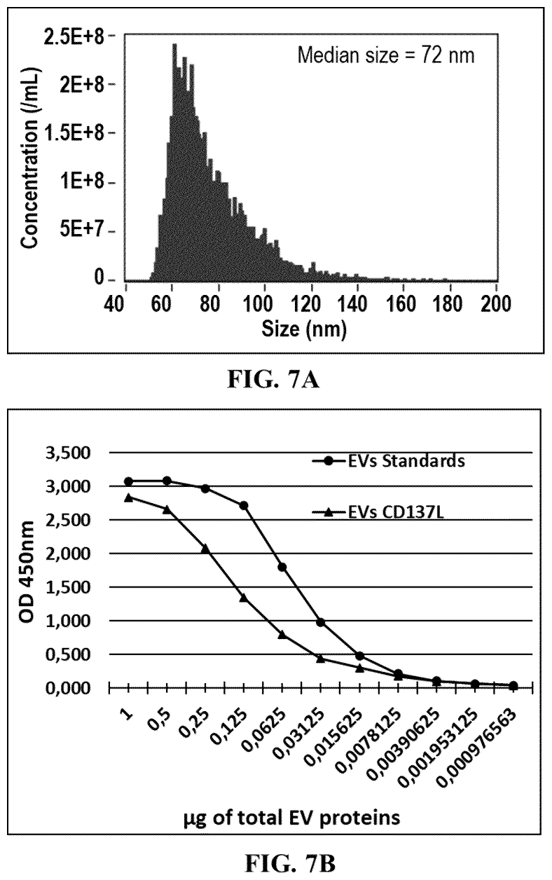

FIG. 7A shows the concentration and size distribution of extracellular vesicles harboring CD137L protein. Extracellular vesicles harboring CD137L protein were analyzed using a Nanoanalyzer equipment (NanoFcm brand) to obtain the concentration and the size distribution of CD137L-EVs. As shown of FIG. 7A, extracellular vesicles harboring CD137L protein exhibit the median size of extracellular vesicles: 72 nm.

In addition, FIG. 7B shows the detection by ELISA of CD81 marker (i.e., a marker specific for extracellular vesicles). As shown on FIG. 7B, extracellular vesicles harboring CD137L protein exhibit the same amount of CD81 marker as compared to extracellular vesicles obtained from untransfected cells (EV standards).

### OX40L Construct Expression

HEK293T cells were stably transfected with DNA encoding an OX40L construct comprising a pilot peptide fused upstream of OX40L (encoding the polypeptide of SEQ ID NO: 91), to express the fusion protein and sort it with extracellular vesicles. Cell pellets were prepared and extracellular vesicles were purified from conditioned culture media. Cells and extracellular vesicles were lysed either under reducing conditions or non-reducing (native) conditions and then submitted or not to deglycosylation using PNGase. Lysates were separated by polyacrylamide gel electrophoresis followed by immunotransfert analysis (WB). WB were probed with anti-pilot peptide rabbit polyclonal antibodies.

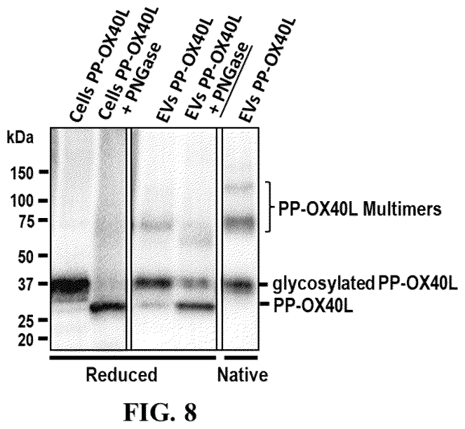

As shown of FIG. 8, OX40L construct was expressed in cells and has an apparent molecular weight of 37 kDa that is lowered to 27 kDa when deglycosylated with PNGase digestion (FIG. 8—Cells PP-OX40L lane and Cells PP-OX40L+PNGase lane). The same glycosylated 37 kDa form is sorted with extracellular vesicles as demonstrated by the lower molecular weight of the protein after PNGase digestion (FIG. 8—EVs PP-OX40L lane and EVs PP-OX40L+PNGase lane). OX40L construct from extracellular vesicles treated under native conditions reveal the presence of higher molecular weight species demonstrating its natural multimerization (FIG. 8—EVs PP-OX40L lane).

1. A chimeric polypeptide comprising:
i) an amino acid sequence of a protein of interest, wherein said protein of interest is selected from a type II transmembrane protein, a protein from the TNF or TNFR superfamily, a protein from the TGF-β superfamily, a protein from the neurotrophin family, and a protein from the CTRP family selected from the group consisting of: CTRP1, CTRP2, CTRP3, CTRP4, CTRP5, CTRP6, CTRP7, CTRP8, CTRP9, CTRP10, CTRP11, CTRP12, CTRP13, CTRP14 and CTRP15;
ii) optionally an amino acid sequence of a transmembrane domain of a transmembrane protein; and
iii) an amino acid sequence of a pilot peptide interacting with the Endosomal Sorting Complexes Required for Transport (ESCRT) cellular machinery and/or an amino acid sequence of a sub-membrane targeting domain,
wherein the components i), ii) and iii) are organized in the chimeric polypeptide from C-terminal to N-terminal.

2. (canceled)

3. The chimeric polypeptide according to claim 1, wherein the protein of interest is selected from the group consisting of: CD137L, OX40L, neuraminidase N2, TRAIL, TNFα, CD30L, TGF-β1, BMP7, BMP9, GDNF, BDNF and CTRP3.

4. The chimeric polypeptide according to claim 1, wherein the sub-membrane targeting domain is linked to an anchoring molecule, and wherein said anchoring molecule is located at the N-terminal position of the chimeric polypeptide.

5. The chimeric polypeptide according to claim 1, further comprising at least one linker between the amino acid sequence of the protein of interest and the amino acid sequence of the transmembrane domain and/or between the amino acid sequence of the transmembrane domain or of the amino acid sequence of the pilot peptide and the amino acid sequence of the sub-membrane targeting domain.

6. The chimeric polypeptide according to claim 1, wherein the transmembrane domain is selected from the transmembrane domain of a type II transmembrane protein, the transmembrane domain of influenza virus neuraminidase transmembrane protein, the transmembrane domain of CD40L, and the transmembrane domain of CD8.

7. The chimeric polypeptide according to claim 1, wherein the pilot peptide comprises at least one YxxL motif or DYxxL motif, and at least one PxxP motif or PPxY motif, in which “x” represents any amino acid residue.

8. The chimeric polypeptide according to claim 1, wherein the pilot peptide comprises an amino acid sequence with SEQ ID NO: 47 or a variant thereof, wherein the variant of SEQ ID NO: 47 retains at least three YxxL and/or DYxxL motifs; and at least four PxxP motifs; wherein “x” represents any amino acid residue.

9. A nucleic acid encoding the chimeric polypeptide according to claim 1.

10. An extracellular vesicle comprising the chimeric polypeptide according to claim 1.

11. An extracellular vesicle comprising the chimeric polypeptide according to claim 1, wherein the extracellular vesicle is an exosome.

12. A population of extracellular vesicles comprising the chimeric polypeptide according to claim 1.

13. An extracellular vesicle comprising the chimeric polypeptide according to claim 1 being purified.

14. A method of treatment comprising a nucleic acid encoding the chimeric polypeptide according to claim 1.

15. A method of treatment of a disease, disorder or condition comprising a nucleic acid encoding the chimeric polypeptide according to claim 1, wherein the treatment of a disease, disorder or condition is selected from the group consisting of: neurodegenerative diseases, auto-immune diseases, viral infections, T-cell immune responses, NK-cell immune responses, homeostatic imbalance associated diseases, diabetes, obesity and associated metabolic disease, insulin resistance, cardiovascular diseases, inflammatory conditions, hormonal cancers and cancer progression.

16. The chimeric polypeptide according to claim 1, wherein the sub-membrane targeting domain is linked to an anchoring molecule, and the anchoring molecule is a fatty acid.

17. The chimeric polypeptide according to claim 1, wherein the transmembrane domain is selected from the transmembrane domain of a type II transmembrane protein, the transmembrane domain of influenza virus neuraminidase transmembrane protein, the transmembrane domain of CD40L, and the transmembrane domain of CD8, and wherein the transmembrane domain of CD40L is as set forth in SEQ ID NO: 17 and the transmembrane domain of CD8 is as set forth in SEQ ID NO: 19.

18. The chimeric polypeptide according to claim 1, wherein the pilot peptide comprises at least one YxxL motif or DYxxL motif, and at least one PxxP motif or PPxY motif, in which “x” represents a proline residue.

19. An extracellular vesicle comprising the chimeric polypeptide according to claim 1, wherein
the transmembrane domain of the chimeric polypeptide is anchored in the extracellular vesicle lipid bilayer; and
the protein of interest of the chimeric polypeptide is exposed at the outer surface of the extracellular vesicle.

20. A population of extracellular vesicles comprising the chimeric polypeptide according to claim 1 and further comprising soluble protein of interest in its cytosol.

21. A method of treatment of a disease, disorder or condition comprising an extracellular vesicle comprising the chimeric polypeptide according to claim 1.
